(19) United States
(12) Patent Application Publication
(10) Pub. No.: US 2018/0163022 A1
(43) Pub. Date: Jun. 14, 2018

(54) NEAR INFRARED ABSORBING CURABLE COMPOSITION, CURED FILM, SOLID IMAGE PICKUP ELEMENT, INFRARED ABSORBER, AND COMPOUND

(71) Applicant: FUJIFILM Corporation, Tokyo (JP)

(72) Inventors: Yuki HIRAI, Haibara-gun (JP); Daisuke SASAKI, Haibara-gun (JP); Yoshihiro JIMBO, Haibara-gun (JP)

(73) Assignee: FUJIFILM Corporation, Tokyo (JP)

(21) Appl. No.: 15/890,577

(22) Filed: Feb. 7, 2018

Related U.S. Application Data
(63) Continuation of application No. PCT/JP2016/070635, filed on Jul. 13, 2016.

(30) Foreign Application Priority Data
Sep. 9, 2015 (JP) 2015-177692
May 30, 2016 (JP) 2016-107394

(51) Int. Cl. C08K 5/435; C08F 220/18; C08F 220/14; C08F 220/32; C08J 3/24; C08K 5/29; C08K 5/3417; C08K 5/47; C07C 311/09; C07D 209/08; C07D 455/04; C07C 251/30; C07D 277/44; G02B 5/20

(52) U.S. Cl. CPC C08K 5/435; H01L 27/1462; C08F 220/14; C08F 220/32; C08J 3/24; C08K 5/29; C08K 5/3417; C08K 5/47; C07C 311/09; C07D 209/08; C07D 455/04; C07C 251/30; C07D 277/44; G02B 5/208; C08F 2800/20; C08F 2220/325; C08J 2333/14; C08J 2333/10; C07C 2601/04; C08F 220/18

(57) ABSTRACT

The near infrared absorbing curable composition includes: a compound represented by Formula (1); and a compound having a crosslinking group. In Formula (1), X¹ and X² each independently represent O, S, or a dicyanomethylene group, and A and B each independently represent a group represented by Formula (2). In Formula (2), a wave line represents a binding site of Formula (1), Y_{S} represents a group having active hydrogen, A1 represents an aromatic hydrocarbon ring or an aromatic heterocycle, R_{Z} represents a substituent, m1 represents an integer of 0 to mA, mA represents an integer representing the maximum number of R_{Z}'s which may be substituted in A1, Y_{S} may be bonded to A1 or R_{Z} to form a ring, and R_{Z} may be bonded to A1 to form a ring.

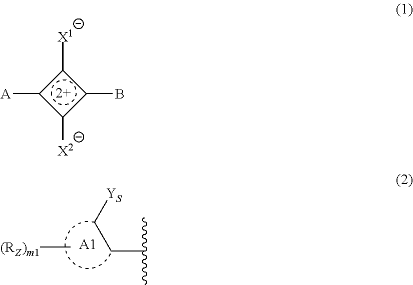

### CROSS-REFERENCE TO RELATED APPLICATIONS

This application is a Continuation of PCT International Application No. PCT/JP2016/70635, filed on Jul. 13, 2016, which claims priority under 35 U.S.C. § 119(a) to Japanese Patent Application No. 2015-177692, filed on Sep. 9, 2015 and Japanese Patent Application No. 2016-107394, filed on May 30, 2016. Each of the above application(s) is hereby expressly incorporated by reference, in its entirety, into the present application.

### BACKGROUND OF THE INVENTION

### 1. Field of the Invention

The present invention relates to a near infrared absorbing curable composition, a cured film, a solid image pickup element, an infrared absorber, and a compound.

### 2. Description of the Related Art

In a video camera, a digital still camera, a mobile phone with a camera function, or the like, a charge coupled device (CCD) or a complementary metal-oxide semiconductor (CMOS), which is a solid image pickup element for a color image, is used. In a light receiving section of this solid image pickup element, a silicon photodiode having sensitivity to infrared light is used. Therefore, it is necessary to correct visibility, and an infrared cut filter is used in many cases.

As a near infrared absorbing compound, for example, a squarylium compound is known.

US2014/0061505A describes that a specific squarylium compound is used for an optical filter.

On the other hand, JP1993-155144A (H5-155144A) describes a technique relating to an infrared heating type thermal transfer recording sheet including a specific squarylium compound as an infrared absorbing material.

In addition, JP2620026B describes a photocurable composition including an ethylenically unsaturated monomer and a squarylium compound represented by one selected from the following Formula (a) and Formula (b) as a sensitizer.

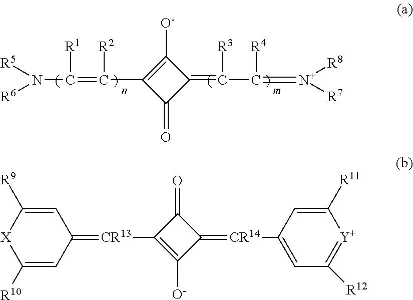

### SUMMARY OF THE INVENTION

An infrared cut filter is required to have excellent infrared shielding properties and visible transparency. In addition, in an infrared cut filter, further improvement of heat resistance and light fastness is required, and it is desired that discoloration caused by heating or light irradiation is suppressed and visible transparency is excellent even after heating or light irradiation.

According to the investigation by the present inventors, it was found that, with the techniques described in US2014/0061505A, JP1993-155144A (H5-155144A), and JP2620026B, it is difficult to manufacture an infrared cut filter in which infrared shielding properties and visible transparency are excellent, discoloration caused by heating or light irradiation is suppressed, and heat resistance and light fastness are excellent.

Accordingly, an object of the present invention is to provide a near infrared absorbing curable composition with which a cured film having excellent infrared shielding properties, visible transparency, heat resistance, and light fastness can be manufactured, a cured film, a solid image pickup element, an infrared absorber, and a compound.

The present inventors performed various investigations and thus found that the object can be achieved by using a near infrared absorbing curable composition including a compound represented by the following Formula (1) and a compound having a crosslinking group, thereby completing the present invention. The present invention provides the following.

<1> A near infrared absorbing curable composition comprising:

a compound represented by Formula (1); and

a compound having a crosslinking group,

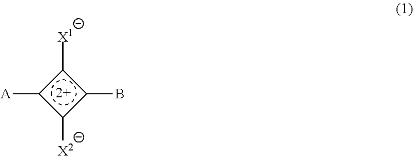

in which in Formula (1), X¹ and X² each independently represent O, S, or a dicyanomethylene group, and A and B each independently represent a group represented by Formula (2), and

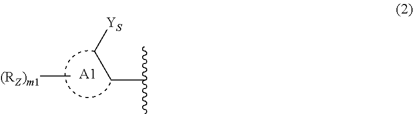

in Formula (2), a wave line represents a binding site of Formula (1), Y_{S} represents a group having active hydrogen, A1 represents an aromatic hydrocarbon ring or an aromatic heterocycle, R_{Z} represents a substituent, m1 represents an integer of 0 to mA, mA represents an integer representing the maximum number of R_{Z}'s which may be substituted in A1, Y_{S} may be bonded to A1 or R_{Z} to form a ring, and R_{Z} may be bonded to A1 to form a ring.

<2> The near infrared absorbing curable composition according to <1>,

in which X¹ and X² represent O.

<3> The near infrared absorbing curable composition according to <1> or <2>,

in which A1 represents a benzene ring, a thiophene ring, a furan ring, a pyrrole ring, a pyridine ring, an azulene ring, or a fused ring including a benzene ring, a thiophene ring, a furan ring, a pyrrole ring, a pyridine ring, or an azulene ring.

<4> The near infrared absorbing curable composition according to any one of <1> to <3>,

in which A1 represents a benzene ring or a naphthalene ring.

<5> The near infrared absorbing curable composition according to any one of <1> to <4>,

in which at least one of A or B is represented by Formula (3), Formula (4), Formula (5), or Formula (6),

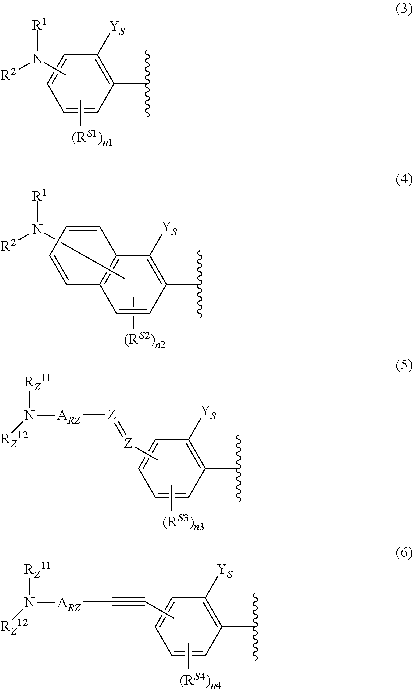

in Formula (3), a wave line represents a binding site of Formula (1), Y_{S} represents a group having active hydrogen, R¹ and R² each independently represent an alkyl group, an aryl group, or a heteroaryl group, R^{S1} represents a substituent, n1 represents an integer of 0 to 3, and R¹ and R² may be bonded to each other to form a ring or may be bonded to a benzene ring bonded to Y_{S} to form a ring,

in Formula (4), a wave line represents a binding site of Formula (1), Y_{S} represents a group having active hydrogen, R^{S2}'s each independently represent a substituent, n2 represents an integer of 0 to 5, and R¹ and R² may be bonded to each other to form a ring or may be bonded to a naphthalene ring bonded to Y_{S} to form a ring,

in Formula (5), a wave line represents a binding site of Formula (1), Y_{S} represents a group having active hydrogen, Z represents CR or N, R represents a hydrogen atom, an alkyl group, a halogen atom, or a cyano group, A_{RZ} represents an aromatic hydrocarbon ring or an aromatic heterocycle, R_{Z}¹¹ and R_{Z}¹² each independently represent a hydrogen atom, an alkyl group, an alkenyl group, an alkynyl group, an aryl group, a heteroaryl group, or an aralkyl group, R_{Z}¹¹ and R_{Z}¹² may be bonded to each other to form a ring, R^{S3} represents a substituent, and n3 represents an integer of 0 to 3, and

in Formula (6), a wave line represents a binding site of Formula (1), Y_{S} represents a group having active hydrogen, A_{RZ} represents an aromatic hydrocarbon ring or an aromatic heterocycle, R_{Z}¹¹ and R_{Z}¹² each independently represent a hydrogen atom, an alkyl group, an alkenyl group, an alkynyl group, an aryl group, a heteroaryl group, or an aralkyl group, R_{Z}¹¹ and R_{Z}¹² may be bonded to each other to form a ring, R^{S4} represents a substituent, and n4 represents an integer of 0 to 3.

<6> The near infrared absorbing curable composition according to any one of <1> to <5>,

in which at least one of A or B is represented by Formula (3-1), Formula (5-1), or Formula (6-1),

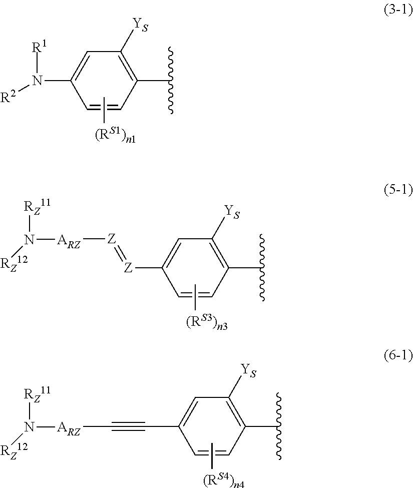

in Formula (3-1), a wave line represents a binding site of Formula (1), Y_{S} represents a group having active hydrogen, R¹ and R² each independently represent an alkyl group, an aryl group, or a heteroaryl group, R^{S1} represents a substituent, n1 represents an integer of 0 to 3, and R¹ and R² may be bonded to each other to form a ring or may be bonded to a benzene ring bonded to Y_{S} to form a ring,

in Formula (5-1), a wave line represents a binding site of Formula (1), Y_{S} represents a group having active hydrogen, Z represents CR or N, R represents a hydrogen atom, an alkyl group, a halogen atom, or a cyano group, A_{RZ} represents an aromatic hydrocarbon ring or an aromatic heterocycle, R_{Z}¹¹ and R_{Z}¹² each independently represent a hydrogen atom, an alkyl group, an alkenyl group, an alkynyl group, an aryl group, a heteroaryl group, or an aralkyl group, R_{Z}¹¹ and R_{Z}¹² may be bonded to each other to form a ring, R^{S3} represents a substituent, and n3 represents an integer of 0 to 3, and

in Formula (6-1), a wave line represents a binding site of Formula (1), Y_{S} represents a group having active hydrogen, A_{RZ} represents an aromatic hydrocarbon ring or an aromatic heterocycle, R_{Z}¹¹ and R_{Z}¹² each independently represent a hydrogen atom, an alkyl group, an alkenyl group, an alkynyl group, an aryl group, a heteroaryl group, or an aralkyl group, R_{Z}¹¹ and R_{Z}¹² may be bonded to each other to form a ring, R^{S4} represents a substituent, and n4 represents an integer of 0 to 3.

<7> The near infrared absorbing curable composition according to any one of <1> to <6>,

in which at least one of A or B is represented by Formula (3-1-1) or Formula (3-1-2),

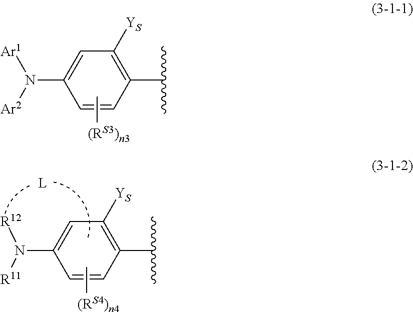

in Formula (3-1-1), a wave line represents a binding site of Formula (1), Y_{S} represents a group having active hydrogen, Ar¹ and Ar² each independently represent an aryl group or a heteroaryl group, R^{S11} represents a substituent, n11 represents an integer of 0 to 2, and Ar¹ and Ar² may be bonded to each other to form a ring or may be bonded to a benzene ring bonded to Y_{S} to form a ring, and

in Formula (3-1-2), a wave line represents a binding site of Formula (1), Y_{S} represents a group having active hydrogen, R¹¹ represents an alkyl group, an aryl group, or a heteroaryl group, R¹² represents an alkylene group, L represents a divalent linking group through which R¹² and a benzene ring are bonded to form a ring, R^{S12} represents a substituent, n12 represents an integer of 0 to 2, and may be bonded to a benzene ring bonded to Y_{S} to form a ring.

<8> The near infrared absorbing curable composition according to <7>,

in which at least one of A or B is represented by Formula (3-1-1).

<9> The near infrared absorbing curable composition according to any one of <1> to <8>,

in which Y_{S} is represented by Formula (Y-1),

—W—Z (Y-1),

W represents a single bond or a divalent linking group,

Z represents —OH, —NHCOR^{x1}, —NHCONR^{x1}R^{x2}, —NHCOOR^{x1}, —NHSO₂R^{x1}, or —NHBR^{x1}R^{x2},

R^{x1} and R^{x2} each independently represent a substituent, and

R^{x1} and R^{x2} may be bonded to each other to form a ring or may be bonded to an aromatic hydrocarbon ring or an aromatic heterocycle bonded to Y_{S} to form a ring.

<10> The near infrared absorbing curable composition according to any one of <1> to <9>,

in which Y_{S} is represented by Formula (Y-2), and

—NH-T (Y-2),

T represents a group having a Hammett substituent constant σp value of 0.3 or higher.

<11> The near infrared absorbing curable composition according to <10>,

in which T represents —CO—R^{x3}, —CONH—R^{x3}, —COO—R^{x3}, or —SO₂—R^{x3}, and

R^{x3} represents a substituent.

<12> The near infrared absorbing curable composition according to <10>,

in which T represents —SO₂—R^{x3}, and

R^{x3} represents a substituent.

<13> The near infrared absorbing curable composition according to <11> or <12>,

in which R^{x3} represents a group having a fluorine atom.

<14> The near infrared absorbing curable composition according to any one of <1> to <13>,

in which the compound represented by Formula (1) is a compound represented by Formula (1A),

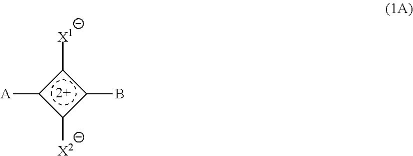

in Formula (1A), X¹ and X² each independently represent O, S, or a dicyanomethylene group, A and B each independently represent a group represented by Formula (2), and at least one of A or B represents a group represented by Formula (10),

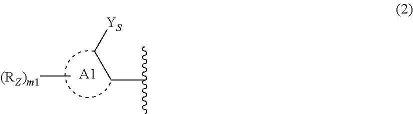

in Formula (2), a wave line represents a binding site of Formula (1A), Y_{S} represents a group having active hydrogen, A1 represents an aromatic hydrocarbon ring or an aromatic heterocycle, R_{Z} represents a substituent, m1 represents an integer of 0 to mA, mA represents an integer representing the maximum number of R_{Z}'s which may be substituted in A1, Y_{S} may be bonded to A1 or R_{Z} to form a ring, and R_{Z} may be bonded to A1 to form a ring, and

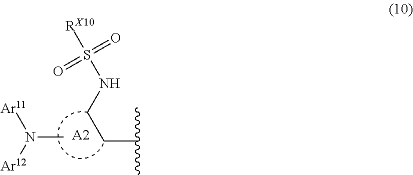

in Formula (10), a wave line represents a binding site of Formula (1A), A2 represents an aromatic hydrocarbon ring or an aromatic heterocycle, Ar¹¹ and Ar¹² each independently represent an aryl group or a heteroaryl group, R^{X10} represents a substituent, and Ar¹¹ and Ar¹² may be bonded to each other to form a ring or may be bonded to A2 to form a ring.

<15> The near infrared absorbing curable composition according to any one of <1> to <14>,

in which the compound having a crosslinking group is at least one selected from the group consisting of a compound which has a group having an ethylenically unsaturated bond, a compound having a cyclic ether group, a compound having an alkoxysilyl group, and a compound having a chlorosilyl group.

<16> The near infrared absorbing curable composition according to any one of <1> to <15>, further comprising:

at least one selected from the group consisting of a polyfunctional thiol, an alcohol, an amine, and a carboxylic acid.

<17> A cured film which is formed using the near infrared absorbing curable composition according to any one of <1> to <16>.

<18> The cured film according to <17>, which is an infrared cut filter.

<19> A solid image pickup element comprising:
the cured film according to <17>.

<20> An infrared absorber which is represented by Formula (1A),

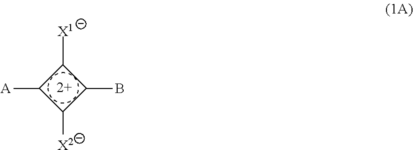

in Formula (1A), X¹ and X² each independently represent O, S, or a dicyanomethylene group, A and B each independently represent a group represented by Formula (2), and at least one of A or B represents a group represented by Formula (10),

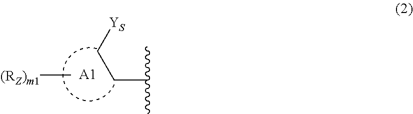

in Formula (2), a wave line represents a binding site of Formula (1A), Y_{S} represents a group having active hydrogen, A1 represents an aromatic hydrocarbon ring or an aromatic heterocycle, R_{Z} represents a substituent, m1 represents an integer of 0 to mA, mA represents an integer representing the maximum number of R_{Z}'s which may be substituted in A1, Y_{S} may be bonded to A1 or R_{Z} to form a ring, and R_{Z} may be bonded to A1 to form a ring, and

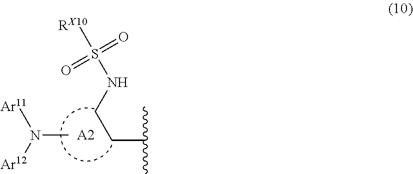

in Formula (10), a wave line represents a binding site of Formula (1A), A2 represents an aromatic hydrocarbon ring or an aromatic heterocycle, Ar¹¹ and Ar¹² each independently represent an aryl group or a heteroaryl group, R^{X10} represents a substituent, and Ar¹¹ and Ar¹² may be bonded to each other to form a ring or may be bonded to A2 to form a ring.

<21> A compound which is represented by Formula (1A),

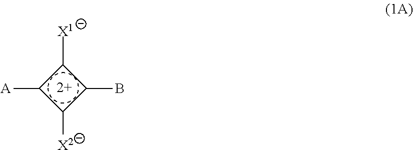

in Formula (1A), X¹ and X² each independently represent O, S, or a dicyanomethylene group, A and B each independently represent a group represented by Formula (2), and at least one of A or B represents a group represented by Formula (10),

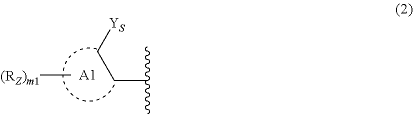

in Formula (2), a wave line represents a binding site of Formula (1A), Y_{S} represents a group having active hydrogen, A1 represents an aromatic hydrocarbon ring or an aromatic heterocycle, R_{Z} represents a substituent, m1 represents an integer of 0 to mA, mA represents an integer representing the maximum number of R_{Z}'s which may be substituted in A1, Y_{S} may be bonded to A1 or R_{Z} to form a ring, and R_{Z} may be bonded to A1 to form a ring, and

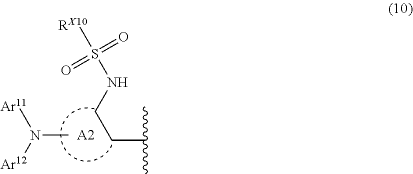

in Formula (10), a wave line represents a binding site of Formula (1A), A2 represents an aromatic hydrocarbon ring or an aromatic heterocycle, Ar¹¹ and Ar¹² each independently represent an aryl group or a heteroaryl group, R^{X10} represents a substituent, and Ar¹¹ and Ar¹² may be bonded to each other to form a ring or may be bonded to A2 to form a ring.

<22> The compound according to <21>,

in which R^{X10} represents a group having a fluorine atom.

According to the present invention, a near infrared absorbing curable composition can be provided, with which a cured film having excellent infrared shielding properties, visible transparency, heat resistance, and light fastness can be manufactured. In addition, a cured film having the above-described properties, a solid image pickup element, an infrared absorber, and a compound can be provided.

### DESCRIPTION OF THE PREFERRED EMBODIMENTS

In this specification, a total solid content denotes the total mass of components of a composition excluding a solvent. In addition, a solid content denotes a solid content at 25° C.

In this specification, unless specified as a substituted group or as an unsubstituted group, a group (atomic group) denotes not only a group having no substituent but also a group having a substituent. For example, “alkyl group” denotes not only an alkyl group having no substituent (unsubstituted alkyl group) but also an alkyl group having a substituent (substituted alkyl group).

In this specification, “radiation” denotes, for example, a bright light spectrum of a mercury lamp, a far ultraviolet ray represented by excimer laser, an extreme ultraviolet ray (EUV ray), an X-ray, or an electron beam. In addition, in the present invention, “light” denotes an actinic ray or radiation. In this specification, unless specified otherwise, “exposure” denotes not only exposure using a bright light spectrum of a mercury lamp, a far ultraviolet ray represented by excimer laser, an X-ray, an EUV ray, or the like but also drawing using a corpuscular beam such as an electron beam or an ion beam.

In this specification, “near infrared light” denotes light (electromagnetic wave) in a wavelength range of 700 to 2500 nm.

In this specification, “(meth)acrylate” denotes either or both of acrylate or methacrylate, “(meth)allyl” denotes either or both of allyl and methallyl, “(meth)acryl” denotes either or both of acryl and methacryl, and “(meth)acryloyl” denotes either or both of acryloyl and methacryloyl.

In this specification, the term “step” denotes not only an individual step but also a step which is not clearly distinguishable from another step as long as an effect expected from the step can be achieved.

In this specification, a weight-average molecular weight and a number-average molecular weight are defined as values in terms of polystyrene obtained by gel permeation chromatography (GPC).

<Near Infrared Absorbing Curable Composition>

A near infrared absorbing curable composition according to the present invention (hereinafter, also referred to as “composition according to the present invention”) includes a compound represented by Formula (1) described below and a compound having a crosslinking group.

The compound represented by Formula (1) described below (hereinafter, also referred to as “squarylium compound (1)”) has a group Y_{S} having active hydrogen at the ortho position of an aromatic hydrocarbon ring or an aromatic heterocycle represented by A1. By using the squarylium compound (1) having the above-described structure, a cured film having excellent infrared shielding properties and visible transparency can be manufactured. In addition, by using the squarylium compound (1) and the compound having a crosslinking group in combination, a cured film can be manufactured in which heat resistance and light fastness are improved, discoloration caused by heating or light irradiation is suppressed, and visible transparency is excellent even after heating or light irradiation. The reason why excellent heat resistance can be obtained is presumed that the glass transition temperature of the film is improved by crosslinking. In addition, the reason why excellent light fastness can be obtained is presumed that the oxygen permeability of the film deteriorates due to crosslinking. Further, the solvent resistance of the obtained cured film can improved, and the cured film can be manufactured by multiple coating. Therefore, for example, the thickness of the film can increase. Further, since the solvent resistance of the obtained cured film is improved, another film such as a protective film can be formed on a surface of the cured film manufactured using the composition according to the present invention.

Hereinafter, each component of the composition according to the present invention will be described.

<<Compound Represented by Formula (1) (Squarylium Compound (1))>>

The composition according to the present invention includes the compound represented by Formula (1) (squarylium compound (1)). In the present invention, the squarylium compound (1) has an absorption maximum preferably in a range of 600 to 1200 nm and more preferably in a range of 700 to 1000 nm. By adjusting the absorption maximum to be in the above-described range, a cured film having excellent infrared shielding properties and visible transparency is likely to be manufactured.

In the composition according to the present invention, the content of the squarylium compound (1) is preferably 0.1 to 70 mass % with respect to the total solid content of the composition according to the present invention. The lower limit is preferably 0.5 mass % or higher and more preferably 1.0 mass % or higher. The upper limit is preferably 60 mass % or lower, and more preferably 50 mass % or lower. By adjusting the content to be in the above-described range, excellent infrared absorption capacity can be imparted. In a case where the composition according to the present invention includes two or more squarylium compounds (1), it is preferable that the total content of the squarylium compounds (1) is in the above-described range.

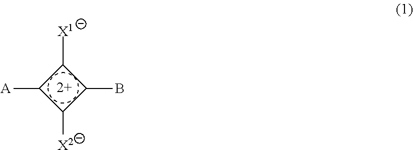

X¹ and X² each independently represent O, S, or a dicyanomethylene group, and A and B each independently represent a group represented by Formula (2).

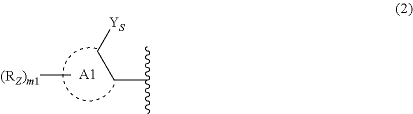

In Formula (2), a wave line represents a binding site of Formula (1), Y_{S} represents a group having active hydrogen, A1 represents an aromatic hydrocarbon ring or an aromatic heterocycle, R_{Z} represents a substituent, m1 represents an integer of 0 to mA, mA represents an integer representing the maximum number of R_{Z}'s which may be substituted in A1, Y_{S} may be bonded to A1 or R_{Z} to form a ring, and R_{Z} may be bonded to A1 to form a ring.

In Formula (1), X¹ and X² each independently represent O, S, or a dicyanomethylene group. From the viewpoint of visible transparency, it is preferable that X¹ and X² represent O.

In Formula (2), A1 represents an aromatic hydrocarbon ring or an aromatic heterocycle.

The number of carbon atoms constituting the aromatic hydrocarbon ring is preferably 6 to 48, more preferably 6 to 22, and still more preferably 6 to 12. The aromatic hydrocarbon ring is preferably a monocycle or a fused ring, more preferably a monocycle or a fused ring composed of 2 to 8 rings, still more preferably a monocycle or a fused ring composed of 2 to 4 rings, even still more preferably a monocycle or a fused ring composed of 2 or 3 rings, and even yet still more preferably a monocycle or a fused ring composed of 2 rings.

It is preferable that the aromatic heterocycle is a 5- or 6-membered ring. In addition, the aromatic heterocycle is preferably a monocycle or a fused ring, more preferably a monocycle or a fused ring composed of 2 to 8 rings, still more preferably a monocycle or a fused ring composed of 2 to 4 rings, even still more preferably a monocycle or a fused ring composed of 2 or 3 rings, and even yet still more preferably a monocycle or a fused ring composed of 2 rings. Examples of a heteroatom included in the aromatic heterocycle include a nitrogen atom, an oxygen atom, and a sulfur atom. Among these, a nitrogen atom or a sulfur atom is preferable. The number of heteroatoms constituting the aromatic heterocycle is preferably 1 to 3 and more preferably 1 or 2.

It is preferable that A1 represents a benzene ring, a thiophene ring, a furan ring, a pyrrole ring, a pyridine ring, an azulene ring, or a fused ring including one of the above-described rings. Examples of the fused ring include a naphthalene ring, a benzothiophene ring, a benzofuran ring, an isobenzofuran ring, a benzimidazole ring, an indole ring, an isoindole ring, a quinoline ring, an isoquinoline ring, a thienopyrrole ring, and a pyrrolothiazole ring. In the present invention, it is preferable that A1 represents a benzene ring or a naphthalene ring. From the viewpoints of visible transparency, light fastness, and heat resistance, it is more preferable that A represents a benzene ring.

In Formula (2), Y_{S} represents a group having active hydrogen. In the present invention, the group having active hydrogen represented by Y_{S} refers to a group which can form a hydrogen bond to X¹ and X² in Formula (1). In a case where A1 represents an aromatic heterocycle having an active hydrogen at the ortho position (for example, a pyrrole ring, a thienopyrrole ring, or a pyrrolothiazole ring), a hydrogen atom positioned at the ortho position of the aromatic heterocycle corresponds to Y_{S}.

In the present invention, Y_{S} may be bonded to A1 or R_{Z} to form a ring. Examples of the ring which is formed by Y_{S} being bonded to A1 or R_{Z} include an alicyclic ring (a nonaromatic hydrocarbon ring), an aromatic ring, and a heterocycle. The ring may be a monocycle or a polycycle. In a case where Y_{S} is bonded to A1 or R_{Z} to form a ring through a linking group, it is preferable that the linking group is a divalent linking group selected from the group consisting of —CO—, —O—, —NH—, an alkylene group having 1 to 10 carbon atoms, and a combination thereof.

It is preferable that the group having active hydrogen represented by Y_{S} is a group represented by Formula (Y-1).

—W—Z (Y-1)

In Formula (Y-1), W represents a single bond or a divalent linking group. Examples of the divalent linking group include an alkylene group, an arylene group, —O—, —NR′—(R′ represents a hydrogen atom, an alkyl group which may have a substituent, or an aryl group which may have a substituent and preferably represents a hydrogen atom), —SO₂—, —CO—, —O—, —S—, and a combination thereof. It is preferable that W represents a single bond.

In Formula (Y-1), for example, Z represents —OH, —SH, —COOH, —SO₃H, —NHR^{x1}, —NR^{x1}R^{x2}, —NHCOR^{x1}, —CONR^{x1}R^{x2}, —NHCONR^{x1}R^{x2}, —NHCOOR^{x1}, —NHSO₂R^{x1}, —B(OH)₂, —PO(OH)₃, or —NHBR^{x1}R^{x2}. As Z, —OH, —NHCOR^{x1}, —NHCONR^{x1}R^{x2}, —NHCOOR^{x1}, —NHSO₂R^{x1}, or —NHBR^{x1}R^{x2} is preferable, —NHCOR^{x1}, —NHCONR^{x1}R^{x2}, —NHCOOR^{x1}, or —NHSO₂R^{x1} is more preferable, —NHCOR^{x1} or —NHSO₂R^{x1} is still more preferable, and —NHSO₂R^{x1} is even still more preferable.

R^{x1} and R^{x2} each independently represent a substituent. Examples of the substituent include an alkyl group and an aryl group. Among these, an alkyl group is preferable. The number of carbon atoms in the alkyl group is preferably 1 to 20, more preferably 1 to 15, still more preferably 1 to 8, and even still more preferably 1 or 5. The alkyl group may be linear, branched, or cyclic and is preferably linear or branched. The number of carbon atoms in the aryl group is preferably 6 to 30, more preferably 6 to 20, and still more preferably 6 to 12.

The alkyl group and the aryl group may have a substituent or may be unsubstituted, and preferably has a substituent. Examples of the substituent include substituents described below regarding R. For example, a halogen atom, an aryl group, or an alkoxy group may be used. From the viewpoints of heat resistance and light fastness, a halogen atom is preferable, and a fluorine atom is more preferable.

As R^{x1} and R^{x2}, a group having a fluorine atom is preferable, an alkyl group having a fluorine atom or an aryl group having a fluorine atom is more preferable, an alkyl group having a fluorine atom is still more preferable, and a perfluoroalkyl group having 1 to 5 carbon atoms is even still more preferable.

R^{x1} and R^{x2} may be bonded to each other to form a ring or may be bonded to an aromatic hydrocarbon ring or an aromatic heterocycle bonded to Y_{S} to form a ring. Examples of the ring include an alicyclic ring (a nonaromatic hydrocarbon ring), an aromatic ring, and a heterocycle. The ring may be a monocycle or a polycycle. In addition, it is preferable that a linking group for forming the ring is a divalent linking group selected from the group consisting of —CO—, —O—, —NH—, an alkylene group having 1 to 10 carbon atoms, and a combination thereof. Here, the aromatic hydrocarbon ring or the aromatic heterocycle bonded to Y_{S} corresponds to A1 in Formula (2).

In the present invention, It is preferable that the group having active hydrogen represented by Y_{S} is a group represented by Formula (Y-2).

—NH-T (Y-2)

T represents a group having a Hammett substituent constant σp value of 0.3 or higher.

The Hammett substituent constant σp value will be described. The Hammett rule is an experimental rule proposed by L. P. Hammett in 1935 in order to quantitatively discuss an effects of a substituent on a reaction or a equilibrium of a benzene derivative. The validity of the Hammett rule is widely admitted nowadays. Substituent constants obtained by the Hammett rule are an σp value and an σm value, and these values can be found in many general documents. For example, the detail can be found in “Lange's Handbook of Chemistry” 12th edition, edited by J. A. Dean, 1979 (McGraw-Hill), “Kagaku no Ryoiki (Journal of Japanese Chemistry) special edition” vol. 122, pp. 96-103, 1979 (Nankodo), and “Chem. Rev.” vol. 91, pp. 165-195, 1991. The document shows that the group having a Hammett substituent constant σp value of 0.3 or higher in the present invention is an electron-withdrawing group. The σp value is preferably 0.35 or higher, more preferably 0.4 or higher, and still more preferably 0.5 or higher. The upper limit of the σp value is preferably 1.0 or lower and more preferably 0.8 or lower. Specific examples of the group having a Hammett substituent constant σp value of 0.3 or higher include —CO—CH₃ (σp value=0.50), —CONH—CH₃₃ (σp value=0.36), —COO—CH₃ (σp value=0.45), and —SO₂—CH₃ (σp value=0.72). The values in the parentheses are representative σp values of the substituents extracted from “Chem. Rev.” vol. 91, pp. 165-195, 1991.

In the present invention, it is preferable that T represents —CO—R^{x3}, —CONH—R^{x3}, —COO—R^{x3}, or —SO₂—R^{x3}. From the viewpoints of heat resistance and light fastness, it is more preferable that T represents —SO₂—R^{x3}. That is, from the viewpoints of heat resistance and light fastness, it is more preferable that Y_{S} represents —NH—SO₂—R^{x3}. R^{x3} represents a substituent. The substituent represented by R^{X3} has the same definition as the substituent described above regarding R^{x1} and R^{x2}. As the substituent, a group having a fluorine atom is preferable, an alkyl group (fluoroalkyl group) having a fluorine atom is more preferable, a perfluoroalkyl group is still more preferable, and a perfluoroalkyl group having 1 to 5 carbon atoms is even still more preferable.

In Formula (2), R_{Z} represents a substituent. Examples of the substituent include a halogen atom, a cyano group, a nitro group, an alkyl group, an alkenyl group, an alkynyl group, an aryl group, a heteroaryl group, an aralkyl group, —OR_{Z}¹, —COR_{Z}¹, —COOR_{Z}¹, —OCOR_{Z}¹, —NR_{Z}¹R_{Z}², —NHCOR_{Z}¹, —CONR_{Z}¹R_{Z}², —NHCONR_{Z}¹R_{Z}², —NHCOOR_{Z}¹, —SR_{Z}¹, —SO₂R_{Z}¹, —SO₂OR_{Z}¹, —NHSO₂R_{Z}¹, and —SO₂NR_{Z}¹R_{Z}². R_{Z}¹ to R_{Z}² each independently represent a hydrogen atom, an alkyl group, an alkenyl group, an alkynyl group, an aryl group, a heteroaryl group, or an aralkyl group, and R_{Z}¹ to R_{Z}² may be bonded to each other to form a ring. In a case where R_{Z}¹ in —COOR_{Z}¹ represents a hydrogen atom (that is, a carboxyl group), the hydrogen atom may be dissociable (that is, a carbonate group) or may be in the form of a salt. In a case where R_{Z}¹ in —SO₂OR_{Z}¹ represents a hydrogen atom (that is, a sulfo group), the hydrogen atom may be dissociable (that is, a sulfonate group) or may be in the form of a salt.

Examples of the halogen atom include a fluorine atom, a chlorine atom, a bromine atom, and an iodine atom.

The number of carbon atoms in the alkyl group is preferably 1 to 20, more preferably 1 to 15, and still more preferably 1 to 8. The alkyl group may be linear, branched, or cyclic and is preferably linear or branched.

The number of carbon atoms in the alkenyl group is preferably 2 to 20, more preferably 2 to 12, and still more preferably 2 to 8. The alkenyl group may be linear, branched, or cyclic and is preferably linear or branched.

The number of carbon atoms in the alkynyl group is preferably 2 to 40, more preferably 2 to 30, and still more preferably 2 to 25. The alkynyl group may be linear, branched, or cyclic and is preferably linear or branched.

The number of carbon atoms in the aryl group is preferably 6 to 30, more preferably 6 to 20, and still more preferably 6 to 12.

An alkyl portion of the aralkyl group is the same as the above-described alkyl group. An aryl portion of the aralkyl group is the same as the above-described aryl group. The number of carbon atoms in the aralkyl group is preferably 7 to 40, more preferably 7 to 30, and still more preferably 7 to 25.

The heteroaryl group is preferably a monocycle or a fused ring, more preferably a monocycle or a fused ring composed of 2 to 8 rings, and still more preferably a monocycle or a fused ring composed of 2 to 4 rings. The number of heteroatoms constituting the ring of the heteroaryl group is preferably 1 to 3. It is preferable that the heteroatoms constituting the ring of the heteroaryl group are a nitrogen atom, an oxygen atom, or a sulfur atom. It is preferable that the heteroaryl group is a 5- or 6-membered ring. The number of carbon atoms constituting the ring of the heteroaryl group is preferably 3 to 30, more preferably 3 to 18, and still more preferably 3 to 12.

The alkyl group, the alkenyl group, the alkynyl group, the aralkyl group, the aryl group, and the heteroaryl group may have a substituent or may be unsubstituted. Examples of the substituent include the groups described above regarding R_{Z}. For example, an alkyl group, an alkoxy group, or an aryl group may be used. In addition, for example, a group represented by Formula (W) described below may also be used.

Examples of the ring which is formed by R_{Z}¹ and R_{Z}² being bonded to each other include an alicyclic ring (a nonaromatic hydrocarbon ring), an aromatic ring, and a heterocycle. The ring may be a monocycle or a polycycle. In a case where R_{Z}¹ and R_{Z}² are bonded to each other to form a ring through a linking group, it is preferable that the linking group is a divalent linking group selected from the group consisting of —CO—, —O—, —NH—, an alkylene group having 1 to 10 carbon atoms, and a combination thereof.

In the present invention R_{Z} may be bonded to A1 to form a ring or may be bonded to Y_{S} to form a ring. That is, R_{Z}¹ and/or R_{Z}² may be bonded to A1 and/or Y_{S} to form a ring. In a case where R_{Z}¹ and/or R_{Z}² is bonded to A1 and/or Y_{S} to form a ring through a linking group, it is preferable that the linking group is a divalent linking group selected from the group consisting of —CO—, —O—, —NH—, an alkylene group having 1 to 10 carbon atoms, and a combination thereof.

In the present invention, it is preferable that R_{Z} is —NR_{Z}¹R_{Z}². It is preferable that R_{Z}¹ and R_{Z}² each independently represent an alkyl group, an aryl group, or a heteroaryl group. From the viewpoints of heat resistance and light fastness, R_{Z}¹ and R_{Z}² each independently represent more preferably an aryl group or a heteroaryl group, and still more preferably an aryl group. The alkyl group, the aryl group, and the heteroaryl group may have a substituent or may be unsubstituted. Examples of the substituent include the groups described above regarding R_{Z}. For example, an alkyl group, an alkoxy group, or an aryl group may be used. In addition, in a case where R_{Z}¹ and R_{Z}² represent an aryl group, it is preferable that the aryl group has a solubilizing group as a substituent. According to the aspect, crystallization during heating can be suppressed, and excellent heat resistance is likely to be obtained. As the solubilizing group, a group represented by the following Formula (W) is preferable.

—S₁₀₀-L₁₀₀-T₁₀₀ (W)

In Formula (W), S₁₀₀ represents a single bond, an arylene group, or a heteroarylene group. L¹⁰⁰ represents an alkylene group, an alkenylene group, an alkynylene group, —O—, —S—, —NR^{L1}—, —CO—, —COO—, —OCO—, —CONR^{L1}—, —NR^{L1}CO—, —SO₂—, —OR^{L2}—, or a group including a combination of the above-described groups, R^{L1} represents a hydrogen atom or an alkyl group, and R^{L2} represents an alkylene group.

T¹⁰⁰ represents an alkyl group, a cyano group, a hydroxyl group, a formyl group, a carboxyl group, an amino group, a thiol group, a sulfo group, a phosphoryl group, a boryl group, a vinyl group, an ethynyl group, an aryl group, a heteroaryl group, a trialkylsilyl group, or a trialkoxysilyl group.

In Formula (W), S¹⁰⁰ represents a single bond, an arylene group, or a heteroarylene group. It is preferable that S¹⁰⁰ represents a single bond.

The arylene group may be a monocycle or a polycycle. It is preferable that the arylene group is a monocycle. The number of carbon atoms in the arylene group is preferably 6 to 20 and more preferably 6 to 12.

The heteroarylene group may be a monocycle or a polycycle. It is preferable that the heteroarylene group is a monocycle. The number of heteroatoms constituting the ring of the heteroarylene group is preferably 1 to 3. It is preferable that the heteroatoms constituting the ring of the heteroarylene group are a nitrogen atom, an oxygen atom, a sulfur atom, or a selenium atom. The number of carbon atoms constituting the ring of the heteroarylene group is preferably 3 to 30, more preferably 3 to 18, and still more preferably 3 to 12.

In Formula (W), it is preferable that L¹⁰⁰ represents an alkylene group, an alkenylene group, an alkynylene group, —O—, —S—, —NR^{L1}—, —COO—, —OCO—, —CONR^{L1}—, —SO₂—, —OR^{L2}—, or a group including a combination of the above-described groups. From the viewpoints of flexibility and solvent solubility, an alkylene group, an alkenylene group, —O—, —OR^{L2}—, or a group including a combination of the above-described groups is more preferable, an alkylene group, an alkenylene group, —O—, or —OR^{L2}— is still more preferable, and an alkylene group, —O—, or —OR^{L2}— is even still more preferable.

The number of carbon atoms in the alkylene group represented by L¹⁰⁰ is preferably 1 to 40. The lower limit is more preferably 3 or more, still more preferably 5 or more, even still more preferably 10 or more, and even yet still more preferably 13 or more. The upper limit is more preferably 35 or less and still more preferably 30 or less. The alkylene group may be linear, branched, or cyclic, and is preferably linear or branched, and is still more preferably branched. For example, the number of branches in the alkylene group is preferably 2 to 10 and more preferably 2 to 8. In a case where the number of branches is in the above-described range, solvent solubility is excellent.

The number of carbon atoms in the alkenylene group or the alkynylene group represented by L¹⁰⁰ is preferably 2 to 40.

For example, the lower limit is more preferably 3 or more, still more preferably 5 or more, even still more preferably 8 or more, and even yet still more preferably 10 or more. The upper limit is more preferably 35 or less and still more preferably 30 or less. The alkenylene group and the alkynylene group may be linear or branched, and is preferably linear or branched, and is still more preferably branched. The number of branches is preferably 2 to 10 and more preferably 2 to 8. In a case where the number of branches is in the above-described range, solvent solubility is excellent.

R^{L1} represents a hydrogen atom or an alkyl group and preferably a hydrogen atom. The number of carbon atoms in the alkyl group is preferably 1 to 20, more preferably 1 to 10, still more preferably 1 to 4, and even still more preferably 1 or 2. The alkyl group may be linear or branched.

R^{L2} represents an alkylene group. The alkylene group represented by R^{L2} has the same definition and the same preferable range as the alkylene group described above regarding L¹.

In Formula (W), T¹⁰⁰ represents an alkyl group, a cyano group, a hydroxyl group, a formyl group, a carboxyl group, an amino group, a thiol group, a sulfo group, a phosphoryl group, a boryl group, a vinyl group, an ethynyl group, an aryl group, a heteroaryl group, a trialkylsilyl group, or a trialkoxysilyl group.

The number of carbon atoms in the alkyl group, the alkyl group having a trialkylsilyl group, or the alkyl group having a trialkoxysilyl group is preferably 1 to 40. The lower limit is more preferably 3 or more, still more preferably 5 or more, even still more preferably 10 or more, and even yet still more preferably 13 or more. The upper limit is more preferably 35 or less and still more preferably 30 or less. The alkyl group may be linear, branched, or cyclic and is preferably linear or branched.

The aryl group and the heteroaryl group have the same definitions and the same preferable ranges as the aryl group and the heteroaryl group described above regarding R_{Z}.

In a case where S¹⁰⁰ represents a single bond, L¹⁰⁰ represents an alkylene group, and T¹⁰⁰ represents an alkyl group in Formula (W), the total number of carbon atoms in L¹⁰⁰ and T¹⁰⁰ is preferably 3 or more, and from the viewpoint of solvent solubility, is more preferably 6 or more, and still more preferably 8 or more. For example, the upper limit is preferably 40 or less and more preferably 35 or less. In addition, in a case where S¹⁰⁰ represents an arylene group or a heteroarylene group, the total number of carbon atoms in L¹⁰⁰ and T¹⁰⁰ is preferably 3 or more, and from the viewpoint of solvent solubility, is more preferably 6 or more, and still more preferably 8 or more. For example, the upper limit is preferably 40 or less and more preferably 35 or less.

In a case where the number of carbon atoms in the L¹⁰⁰-T¹⁰⁰ portion is 3 or more, solvent solubility is excellent, defects derived from insoluble matter or the like can be suppressed, and a uniform and high-quality film can be manufactured. Further, by adjusting the number of carbon atoms in the -L¹⁰⁰-T¹⁰⁰ portion to be 3 or more, crystallinity can be suppressed. In general, in a case where the crystallinity of a compound is high, crystallization of the compound progresses during heating of the film, and absorption properties of the film may change. In the present invention, however, crystallization of the compound can be suppressed, and a variation in the absorption properties of the film after heating can be suppressed.

In a preferable aspect of Formula (W), S¹⁰⁰ represents a single bond, L¹⁰⁰ represents an alkylene group, an alkenylene group, an alkynylene group, —O—, —S—, —NR^{L1}—, —COO—, —OCO—, —CONR^{L1}—, —SO₂—, —OR^{L2}—, or a group including a combination of the above-described groups, and T¹⁰⁰ represents an alkyl group or a trialkylsilyl group. As L¹⁰⁰, an alkylene group, an alkenylene group, —O—, —OR^{L2}—, or a group including a combination of the above-described groups is more preferable, an alkylene group, an alkenylene group, —O—, or —OR^{L2}— is still more preferable, and an alkylene group, —O—, or —OR^{L2}— is even still more preferable. As T¹⁰⁰, an alkyl group is more preferable.

It is preferable that the -L¹⁰⁰-T¹⁰⁰ portion in Formula (W) has a branched alkyl structure. Specifically, it is more preferable that the -L¹⁰⁰-T¹⁰⁰ portion is a branched alkyl group or a branched alkoxy group. The number of branches in the -L¹⁰⁰-T¹⁰⁰ portion is preferably 2 to 10 and more preferably 2 to 8. The number of carbon atoms in the -L¹⁰⁰-T¹⁰⁰ portion is preferably 3 or more, more preferably 6 or more, and still more preferably 8 or more. For example, the upper limit is preferably 40 or less and more preferably 35 or less.

It is preferable that the -L¹⁰⁰-T¹⁰⁰ portion in Formula (W) has asymmetric carbon. According to this aspect, the squarylium compound (1) can include a plurality of optical isomers. As a result, the solvent solubility of the squarylium compound (1) can be further improved. The number of asymmetric carbon atoms is preferably 1 or more. The upper limit of asymmetric carbon atoms is not particularly limited and, for example, is preferably 4 or less.

In the present invention, it is preferable that the substituent represented by R_{Z} is a group represented by the following (R_{Z}-1) or a group represented by the following (R_{Z}-2). According to this aspect, the squarylium compound (1) can be made to have an absorption maximum on a longer wavelength side. For example, the squarylium compound (1) can be made to have an absorption maximum on a longer wavelength side of 700 nm or longer (preferably 800 nm or longer and more preferably 800 to 900 nm).

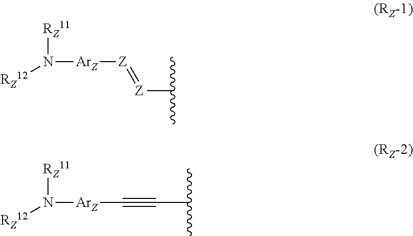

In the formula, Z represents CR or N. R represents a hydrogen atom, an alkyl group, a halogen atom, or a cyano group. A_{RZ} represents an aromatic hydrocarbon ring or an aromatic heterocycle. R_{Z}¹¹ and R_{Z}¹² each independently represent a hydrogen atom, an alkyl group, an alkenyl group, an alkynyl group, an aryl group, a heteroaryl group, or an aralkyl group, and R_{Z}¹¹ and R_{Z}¹² may be bonded to each other to form a ring, A wave line represents a binding site to A1 in Formula (2).

Examples of the aromatic hydrocarbon ring and the aromatic heterocycle represented by A_{RZ} include the aromatic hydrocarbon ring and the aromatic heterocycle described above regarding A1 in Formula (2). Examples of the alkyl group, the alkenyl group, the alkynyl group, the aryl group, the heteroaryl group, and the aralkyl group represented by R_{Z}¹¹ and R_{Z}¹² include the groups described above regarding R_{Z}¹ and R_{Z}², and preferable ranges thereof are also the same. From the viewpoints of heat resistance and light fastness, R_{Z}¹¹ and R_{Z}¹² each independently represent more preferably an aryl group or a heteroaryl group, and still more preferably an aryl group. In addition, the alkyl group, the alkenyl group, the alkynyl group, the aryl group, the heteroaryl group, and the aralkyl group represented by R_{Z}¹¹ and R_{Z}¹² may be unsubstituted or may have a substituent. Examples of the substituent include the substituent and the solubilizing group described above regarding R_{Z}. As the solubilizing group, the group represented by Formula (W) is preferable.

In Formula (2), m1 represents an integer of 0 to mA. mA represents an integer representing the maximum number of R_{Z} which may be substituted in A1. For example, in a case where A1 represents a benzene ring, mA represents 4. In addition, in a case where A1 represents a naphthalene ring, mA represents 6. m1 is preferably an integer of 0 to 4, more preferably 0 to 3, still more preferably 1 to 3, and even still more preferably 1 or 2.

Y_{S} may be bonded to A1 or R_{Z} to form a ring, and R_{Z} may be bonded to A1 to form a ring.

In the present invention, it is preferable that the squarylium compound (1) is a compound in which at least one of A or B in Formula (1) is represented by Formula (3), Formula (4), Formula (5), or Formula (6).

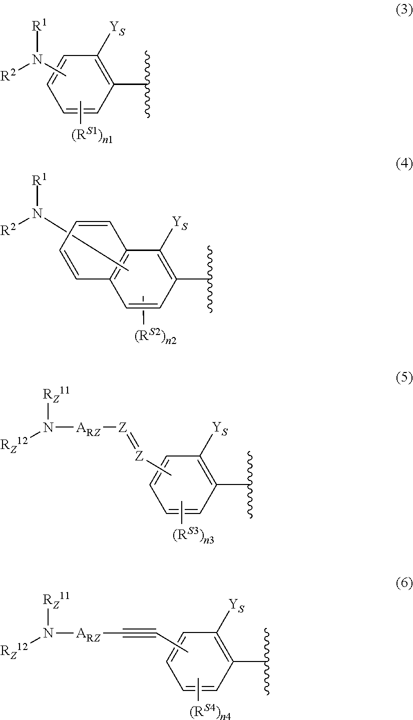

In Formula (3), a wave line represents a binding site of Formula (1), Y_{S} represents a group having active hydrogen, R¹ and R² each independently represent an alkyl group, an aryl group, or a heteroaryl group, R^{S1} represents a substituent, n1 represents an integer of 0 to 3, and R¹ and R² may be bonded to each other to form a ring or may be bonded to a benzene ring bonded to Y_{S} to form a ring.

In Formula (4), a wave line represents a binding site of Formula (1), Y_{S} represents a group having active hydrogen, R^{S2}'s each independently represent a substituent, n2 represents an integer of 0 to 5, and R¹ and R² may be bonded to each other to form a ring or may be bonded to a naphthalene ring bonded to Y_{S} to form a ring.

In Formula (5), a wave line represents a binding site of Formula (1), Y_{S} represents a group having active hydrogen, Z represents CR or N, R represents a hydrogen atom, an alkyl group, a halogen atom, or a cyano group, A_{RZ} represents an aromatic hydrocarbon ring or an aromatic heterocycle, R_{Z}¹¹ and R_{Z}¹² each independently represent a hydrogen atom, an alkyl group, an alkenyl group, an alkynyl group, an aryl group, a heteroaryl group, or an aralkyl group, R_{Z}¹¹ and R_{Z}¹² may be bonded to each other to form a ring, R^{S3} represents a substituent, and n3 represents an integer of 0 to 3.

In Formula (6), a wave line represents a binding site of Formula (1), Y_{S} represents a group having active hydrogen, A_{RZ} represents an aromatic hydrocarbon ring or an aromatic heterocycle, R_{Z}¹¹ and R_{Z}¹² each independently represent a hydrogen atom, an alkyl group, an alkenyl group, an alkynyl group, an aryl group, a heteroaryl group, or an aralkyl group, R_{Z}¹¹ and R_{Z}¹² may be bonded to each other to form a ring, R^{S4} represents a substituent, and n4 represents an integer of 0 to 3.

Y_{S} in Formula (3), Formula (4), Formula (5), and Formula (6) has the same definition and the same preferable range as Y_{S} described above regarding Formula (2). R^{S1} in Formula (3), R^{S2} in Formula (4), R^{S3} in Formula (5), and R^{S4} in Formula (6), each independently represent a substituent. Examples of the substituent include the substituent and the solubilizing group described above regarding R_{Z} in Formula (2). As the solubilizing group, the group represented by Formula (W) is preferable.

R¹ and R² in Formulae (3) and (4) each independently represent an alkyl group, an aryl group, or a heteroaryl group, preferably an alkyl group or an aryl group, and more preferably an aryl group.

The number of carbon atoms in the alkyl group is preferably 1 to 20, more preferably 1 to 15, and still more preferably 1 to 8. The alkyl group may be linear, branched, or cyclic and is preferably linear or branched.

The number of carbon atoms in the aryl group is preferably 6 to 30, more preferably 6 to 20, and still more preferably 6 to 12.

The heteroaryl group is preferably a monocycle or a fused ring, more preferably a monocycle or a fused ring composed of 2 to 8 rings, and still more preferably a monocycle or a fused ring composed of 2 to 4 rings. The number of heteroatoms constituting the ring of the heteroaryl group is preferably 1 to 3. It is preferable that the heteroatoms constituting the ring of the heteroaryl group are a nitrogen atom, an oxygen atom, or a sulfur atom. It is preferable that the heteroaryl group is a 5- or 6-membered ring. The number of carbon atoms constituting the ring of the heteroaryl group is preferably 3 to 30, more preferably 3 to 18, and still more preferably 3 to 12.

The alkyl group, the aryl group, and the heteroaryl group may have a substituent or may be unsubstituted. Examples of the substituent include the groups described above regarding R_{Z}. For example, an alkyl group, an alkoxy group, or an aryl group may be used. In addition, the group represented by Formula (W) is also preferable.

R¹ and R² may be bonded to each other to form a ring and may be bonded to a benzene ring or a naphthalene ring bonded to Y_{S} to form a ring. Examples of the ring include an alicyclic ring, an aromatic ring, and a heterocycle. The ring may be a monocycle or a polycycle. It is preferable that a linking group for forming the ring is a divalent linking group selected from the group consisting of —CO—, —O—, —NH—, an alkylene group having 1 to 10 carbon atoms, and a combination thereof.

Z, A_{RZ}, R_{Z}¹¹, and R_{Z}¹² in Formula (5) have the same definitions and the same preferable ranges as Z, A_{RZ}, R_{Z}¹¹, and R_{Z}¹² described above regarding the groups represented by (R_{Z}-1).

A_{RZ}, R_{Z}¹¹, and R_{Z}¹² in Formula (6) have the same definitions and the same preferable ranges as A_{RZ}, R_{Z}¹¹, and R_{Z}¹² described above regarding the groups represented by (R_{Z}-2).

In Formula (3), n1 represents an integer of 0 to 3, preferably 0 to 2, and more preferably 0 or 1.

In Formula (4), n2 represents an integer of 0 to 5, preferably 0 to 2, and more preferably 0 or 1.

In Formula (5), n3 represents an integer of 0 to 3, preferably 0 to 2, and more preferably 0 or 1.

In Formula (6), n4 represents an integer of 0 to 3, preferably 0 to 2, and more preferably 0 or 1.

In the present invention, in the squarylium compound (1), it is preferable that at least one of A or B in Formula (1) is represented by Formula (3-1), Formula (5-1), or Formula (6-1), it is more preferable that at least one of A or B in Formula (1) is represented by Formula (3-1), and it is still more preferable that both A and B in Formula (1) are represented by Formula (3-1).

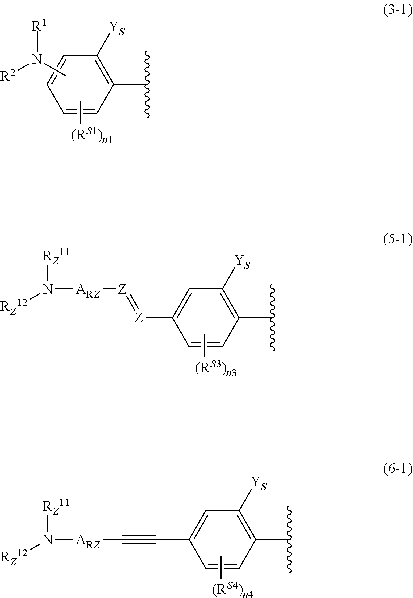

In Formula (3-1), a wave line represents a binding site of Formula (1), Y_{S} represents a group having active hydrogen, R¹ and R² each independently represent an alkyl group, an aryl group, or a heteroaryl group, R^{S1} represents a substituent, n1 represents an integer of 0 to 3, and R¹ and R² may be bonded to each other to form a ring or may be bonded to a benzene ring bonded to Y_{S} to form a ring.

In Formula (5-1), a wave line represents a binding site of Formula (1), Y_{S} represents a group having active hydrogen, Z represents CR or N, R represents a hydrogen atom, an alkyl group, a halogen atom, or a cyano group, A_{RZ} represents an aromatic hydrocarbon ring or an aromatic heterocycle, R_{Z}¹¹ and R_{Z}¹² each independently represent a hydrogen atom, an alkyl group, an alkenyl group, an alkynyl group, an aryl group, a heteroaryl group, or an aralkyl group, R_{Z}¹¹ and R_{Z}¹² may be bonded to each other to form a ring, R^{S3} represents a substituent, and n3 represents an integer of 0 to 3.

in Formula (6-1), a wave line represents a binding site of Formula (1), Y_{S} represents a group having active hydrogen, A_{RZ} represents an aromatic hydrocarbon ring or an aromatic heterocycle, R_{Z}¹¹ and R_{Z}¹² each independently represent a hydrogen atom, an alkyl group, an alkenyl group, an alkynyl group, an aryl group, a heteroaryl group, or an aralkyl group, R_{Z}¹¹ and R_{Z}¹² may be bonded to each other to form a ring, R^{S4} represents a substituent, and n4 represents an integer of 0 to 3.

Y_{S}, R¹, R², R^{S1}, and n1 in Formula (3-1) have the same definitions and the same preferable ranges as Y_{S}, R¹, R², R^{S1}, and n1 described above regarding Formula (3).

Y_{S}, R^{S3}, Z, A_{RZ}, R_{Z}¹¹, R_{Z}¹² and n3 in Formula (5-1) have the same definitions and the same preferable ranges as Y_{S}, R^{S3}, Z, A_{RZ}, R_{Z}¹¹, R_{Z}¹², and n3 described above regarding Formula (5).

Y_{S}, R^{S4}, A_{RZ}, R_{Z}¹¹, R_{Z}¹² and n4 in Formula (6-1) have the same definitions and the same preferable ranges as Y_{S}, R^{S4}, A_{RZ}, R_{Z}¹¹, R_{Z}¹² and n4 described above regarding Formula (6).

In the present invention, in the squarylium compound (1), it is preferable that at least one of A or B in Formula (1) is represented by Formula (3-1-1) or Formula (3-1-2), it is more preferable that at least one of A or B in Formula (1) is represented by Formula (3-1-1), and it is still more preferable that both A and B in Formula (1) are represented by Formula (3-1-1).

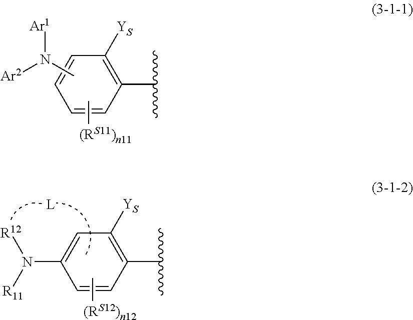

In Formula (3-1-1), a wave line represents a binding site of Formula (1), Y_{S} represents a group having active hydrogen, Ar¹ and Ar² each independently represent an aryl group or a heteroaryl group, R^{S11} represents a substituent, n11 represents an integer of 0 to 2, and Ar¹ and Ar² may be bonded to each other to form a ring or may be bonded to a benzene ring bonded to Y_{S} to form a ring.

In Formula (3-1-2), a wave line represents a binding site of Formula (1), Y_{S} represents a group having active hydrogen, R¹¹ represents an alkyl group, an aryl group, or a heteroaryl group, R¹² represents an alkylene group, L represents a divalent linking group through which R¹² and a benzene ring are bonded to form a ring, R^{S12} represents a substituent, n12 represents an integer of 0 to 2, and R¹¹ may be bonded to a benzene ring bonded to Y_{S} to form a ring.

Y_{S} and R^{S11} in Formula (3-1-1) and Y_{S} and R^{S12} in Formula (3-1-2) have the same definitions and the same preferable ranges as Y_{S} and R^{S1} in Formula (3).

Ar¹ and Ar² in Formula (3-1-1) each independently represent an aryl group or a heteroaryl group, and preferably an aryl group. The details of the aryl group and the heteroaryl group are the same as those of the aryl group and the heteroaryl group described above regarding R¹ and R² in Formula (3). The aryl group and the heteroaryl group may be unsubstituted or may have a substituent. Examples of the substituent include the groups described above regarding R_{Z}. For example, an alkyl group, an alkoxy group, or an aryl group may be used. In addition, it is also preferable that the substituent is the group represented by Formula (W).

Ar¹ and Ar² may be bonded to each other to form a ring and may be bonded to a benzene ring bonded to Y_{S} to form a ring. Examples of the ring include an alicyclic ring, an aromatic ring, and a heterocycle. The ring may be a monocycle or a polycycle. It is preferable that a linking group for forming the ring is a divalent linking group selected from the group consisting of —CO—, —O—, —NH—, an alkylene group having 1 to 10 carbon atoms, and a combination thereof.

R¹¹ in Formula (3-1-2) represents an alkyl group, an aryl group, or a heteroaryl group. The details of the alkyl group, the aryl group, and the heteroaryl group are the same as those of the alkyl group, the aryl group, and the heteroaryl group described above regarding R¹ and R² in Formula (3). The alkyl group, the aryl group, and the heteroaryl group may be unsubstituted or may have a substituent. Examples of the substituent include the groups described above regarding R_{Z}. For example, an alkyl group, an alkoxy group, or an aryl group may be used. In addition, it is also preferable that the substituent is the group represented by Formula (W).

R¹² in Formula (3-1-2) represents an alkylene group. The number of carbon atoms in the alkylene group is preferably 1 to 20, more preferably 1 to 15, and still more preferably 1 to 8. The alkylene group is preferably linear or branched. The alkylene group may be unsubstituted or may have a substituent. Examples of the substituent include the groups described above regarding R_{Z}. For example, an alkyl group, an alkoxy group, or an aryl group may be used.

L in Formula (3-1-2) represents a divalent linking group through which R′² and a benzene ring are bonded to form a ring. Examples of the divalent linking group include a divalent linking group selected from the group consisting of —CO—, —O—, —NH—, an alkylene group having 1 to 10 carbon atoms, and a combination thereof.

n3 in Formula (3-1-1) and n4 in Formula (3-1-2) represents an integer of 0 to 2, preferably 0 or 1, and more preferably 0.

As shown below, cations in Formula (1) are present without localized.

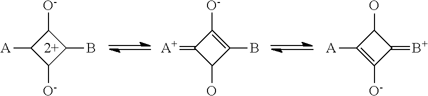

In the present invention, it is preferable that the squarylium compound (1) is a compound represented by Formula (1A). The compound represented by formula (1A) is also the compound according to the present invention.

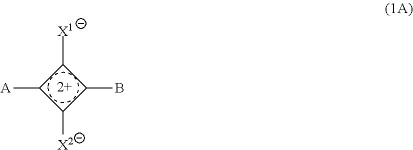

In Formula (1A), X¹ and X² each independently represent O, S, or a dicyanomethylene group, A and B each independently represent a group represented by Formula (2), and at least one of A or B represents a group represented by Formula (10).

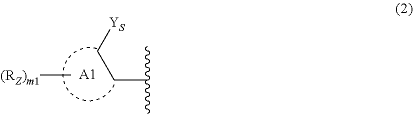

In Formula (2), a wave line represents a binding site of Formula (1A), Y_{S} represents a group having active hydrogen, A1 represents an aromatic hydrocarbon ring or an aromatic heterocycle, R_{Z} represents a substituent, m1 represents an integer of 0 to mA, mA represents an integer representing the maximum number of R_{Z}'s which may be substituted in A1, Y_{S} may be bonded to A1 or R_{Z} to form a ring, and R_{Z} may be bonded to A1 to form a ring.

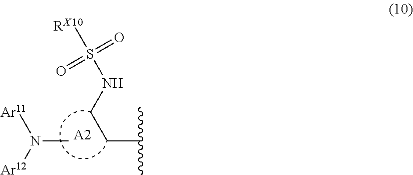

In Formula (10), a wave line represents a binding site of Formula (1A), A2 represents an aromatic hydrocarbon ring or an aromatic heterocycle, Ar¹¹ and Ar¹² each independently represent an aryl group or a heteroaryl group, R^{X10} represents a substituent, and Ar¹¹ and Ar¹² may be bonded to each other to form a ring or may be bonded to A2 to form a ring.

In formula (1A), X¹ and X² each independently represent O, S, or a dicyanomethylene group. It is preferable that X¹ and X² represent O. The group represented by Formula (2) in formula (1A) has the same definition and the same preferable range as the group represented by Formula (2) described above regarding Formula (1).

In Formula (1A), at least one of A or B represents a group represented by Formula (10). From the viewpoints of infrared shielding properties, visible transparency, heat resistance, and light fastness, it is preferable that both A and B represent a group represented by Formula (10).

In Formula (10), A2 represents an aromatic hydrocarbon ring or an aromatic heterocycle. A2 in Formula (10) has the same definition as A1 in Formula (2), and represents preferably a benzene ring or naphthalene ring and more preferably a benzene ring.

Ar¹¹ and Ar¹² in Formula (10) each independently represent an aryl group or a heteroaryl group. The details of the aryl group and the heteroaryl group are the same as those of the aryl group and the heteroaryl group described above regarding R¹ and R² in Formula (3). The aryl group and the heteroaryl group may be unsubstituted or may have a substituent. Examples of the substituent include the groups described above regarding R_{Z}. For example, an alkyl group, an alkoxy group, or an aryl group may be used. In addition, it is also preferable that the substituent is the group represented by Formula (W).

Ar¹¹ and Ar¹² may be bonded to each other to form a ring or may be bonded to A2 to form a ring. Examples of the ring include an alicyclic ring, an aromatic ring, and a heterocycle. The ring may be a monocycle or a polycycle. It is preferable that a linking group for forming the ring is a divalent linking group selected from the group consisting of —CO—, —O—, —NH—, an alkylene group having 1 to 10 carbon atoms, and a combination thereof.

In Formula (10), R^{X10} represents a substituent. Examples of the substituent include an alkyl group and an aryl group. Among these, an alkyl group is preferable. The number of carbon atoms in the alkyl group is preferably 1 to 20, more preferably 1 to 15, still more preferably 1 to 8, and even still more preferably 1 or 5. The alkyl group may be linear, branched, or cyclic and is preferably linear or branched. The number of carbon atoms in the aryl group is preferably 6 to 30, more preferably 6 to 20, and still more preferably 6 to 12.

The alkyl group and the aryl group may have a substituent or may be unsubstituted, and preferably has a substituent. Examples of the substituent include the substituents described regarding R_{Z}. For example, a halogen atom, an aryl group, or an alkoxy group may be used. From the viewpoints of heat resistance and light fastness, a halogen atom is preferable, and a fluorine atom is more preferable.

As R^{X10}, a group having a fluorine atom is preferable, an alkyl group having a fluorine atom or an aryl group having a fluorine atom is more preferable, an alkyl group having a fluorine atom is still more preferable, and a perfluoroalkyl group having 1 to 5 carbon atoms is even still more preferable.

Specific examples of the squarylium compound (1) include compounds shown below, but the squarylium compound (1) is not limited thereto.

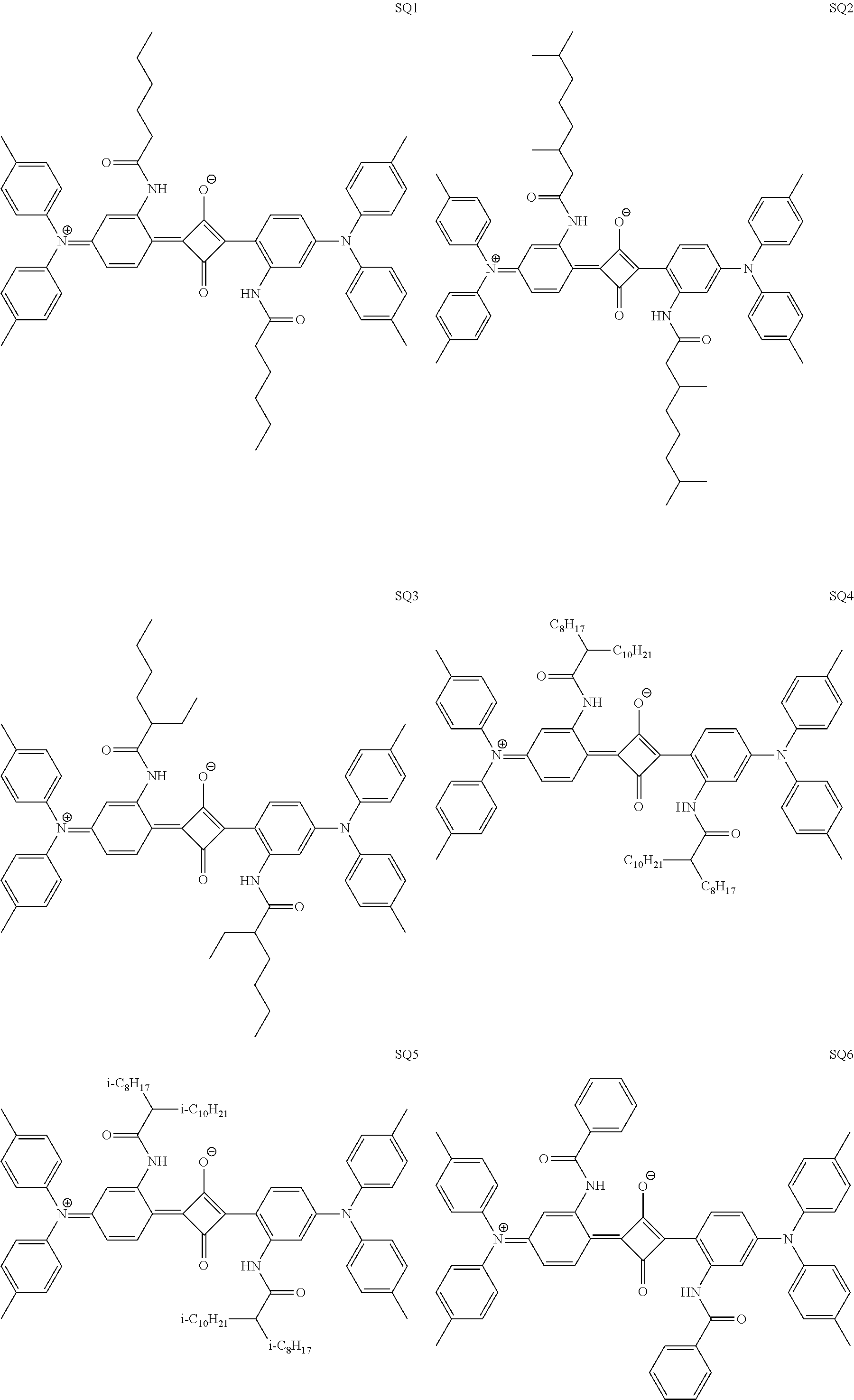

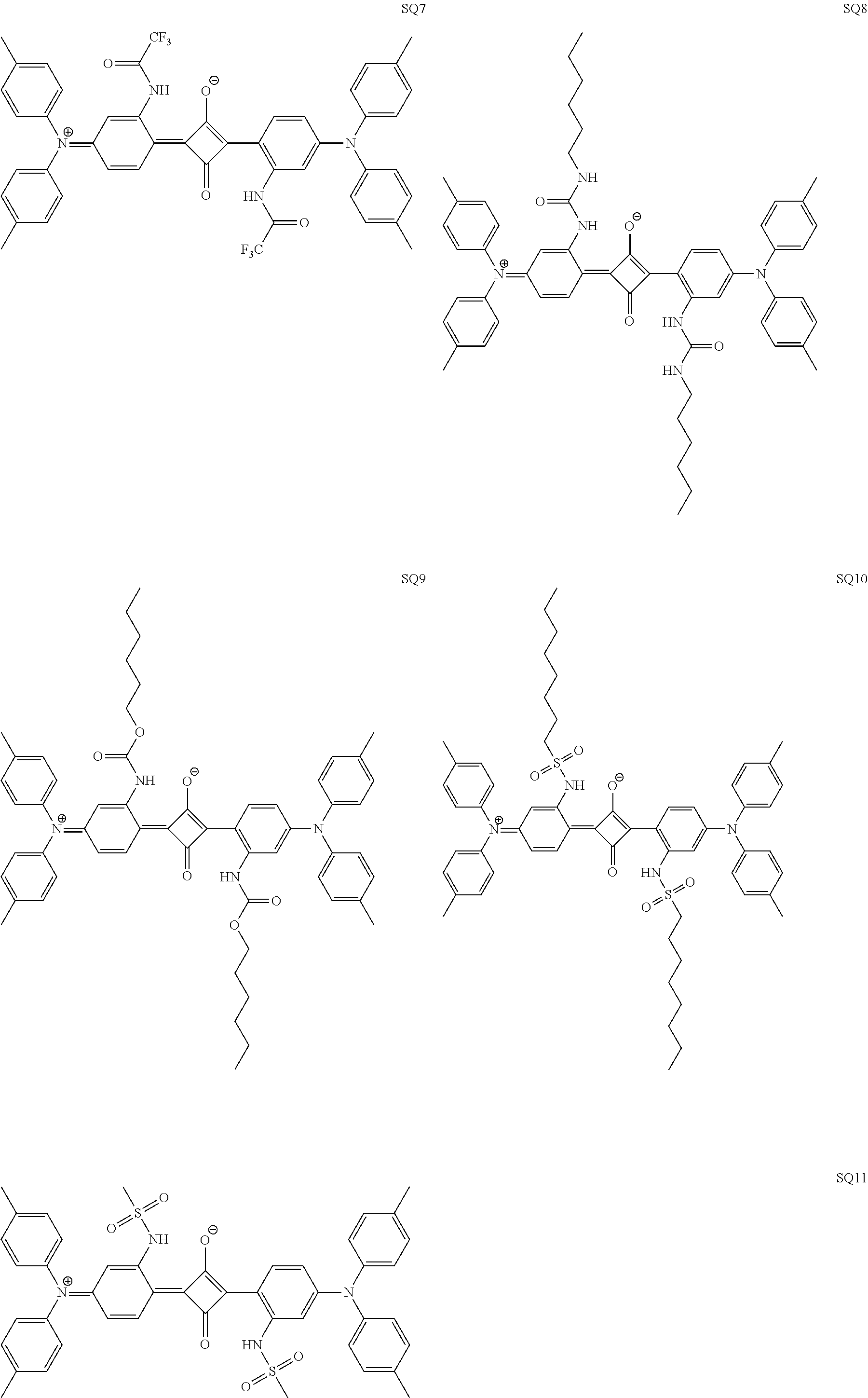

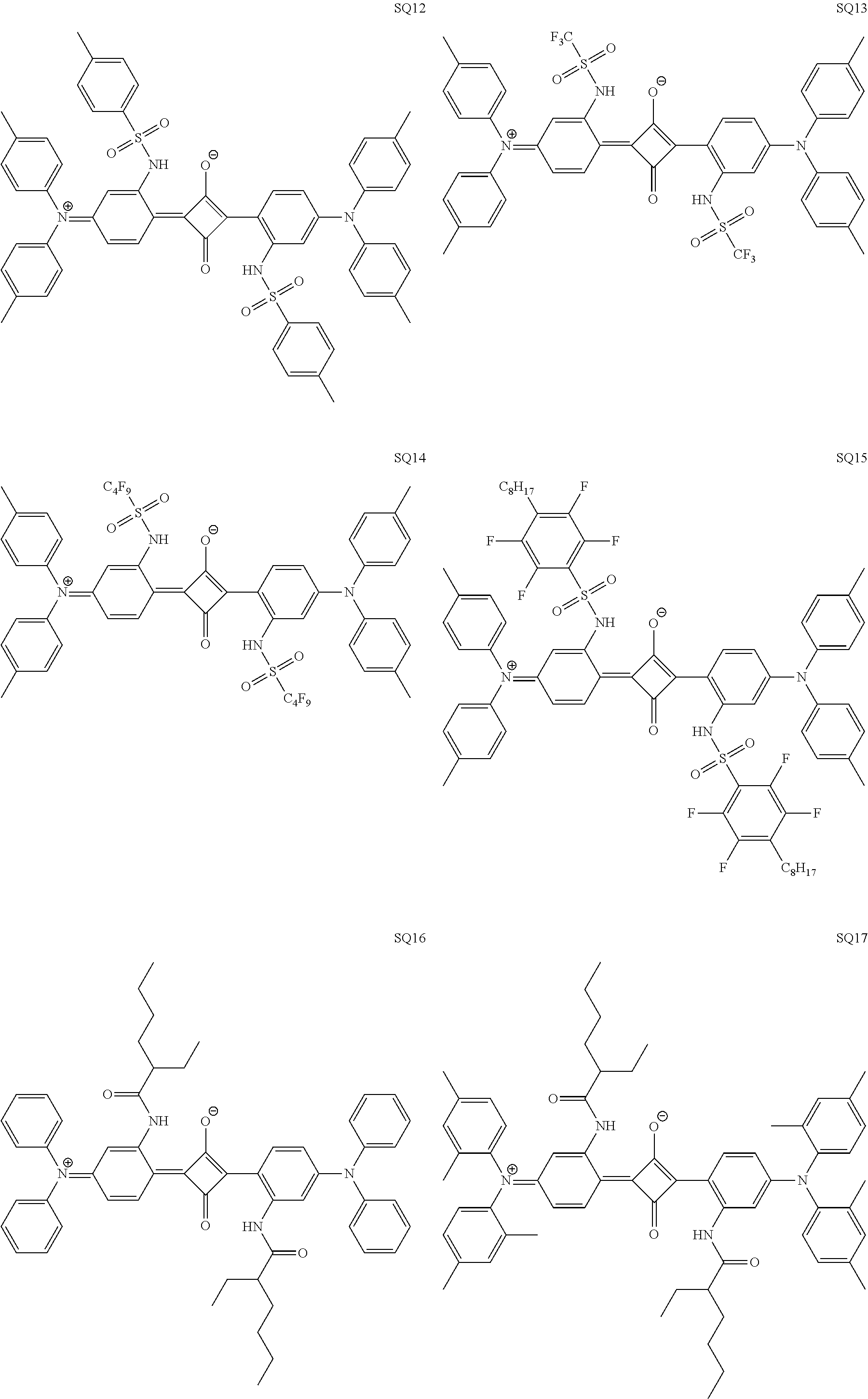

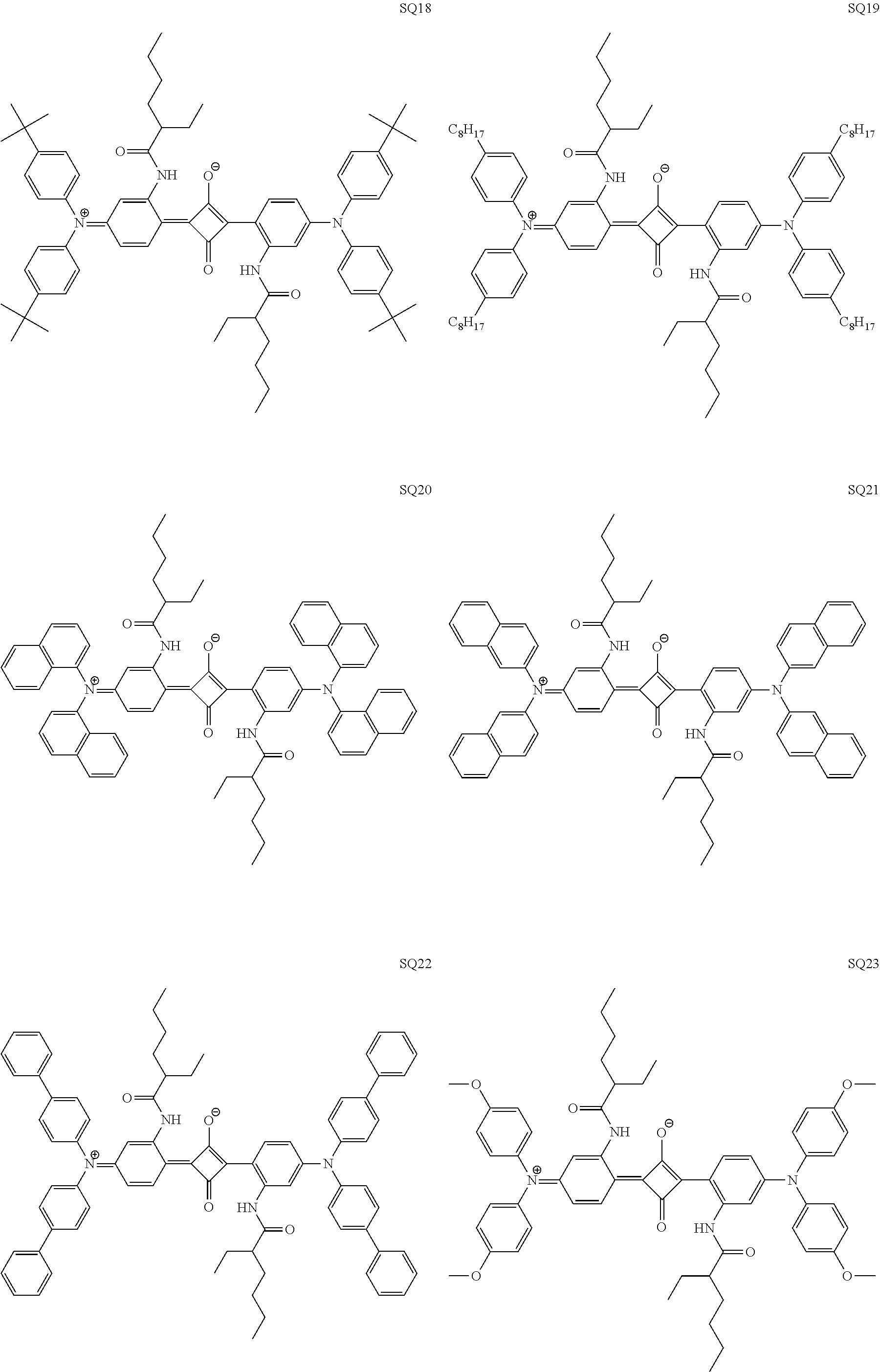

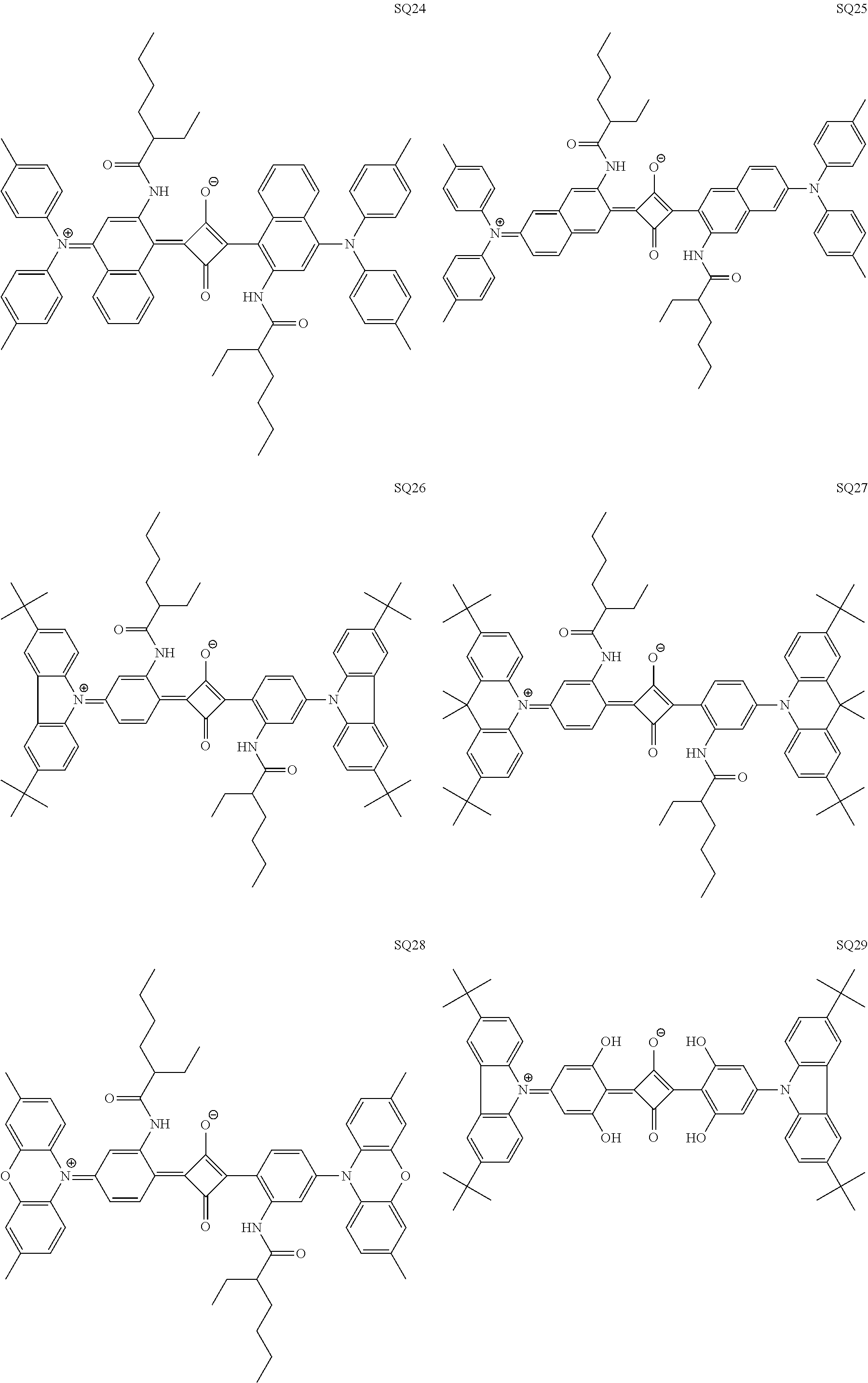

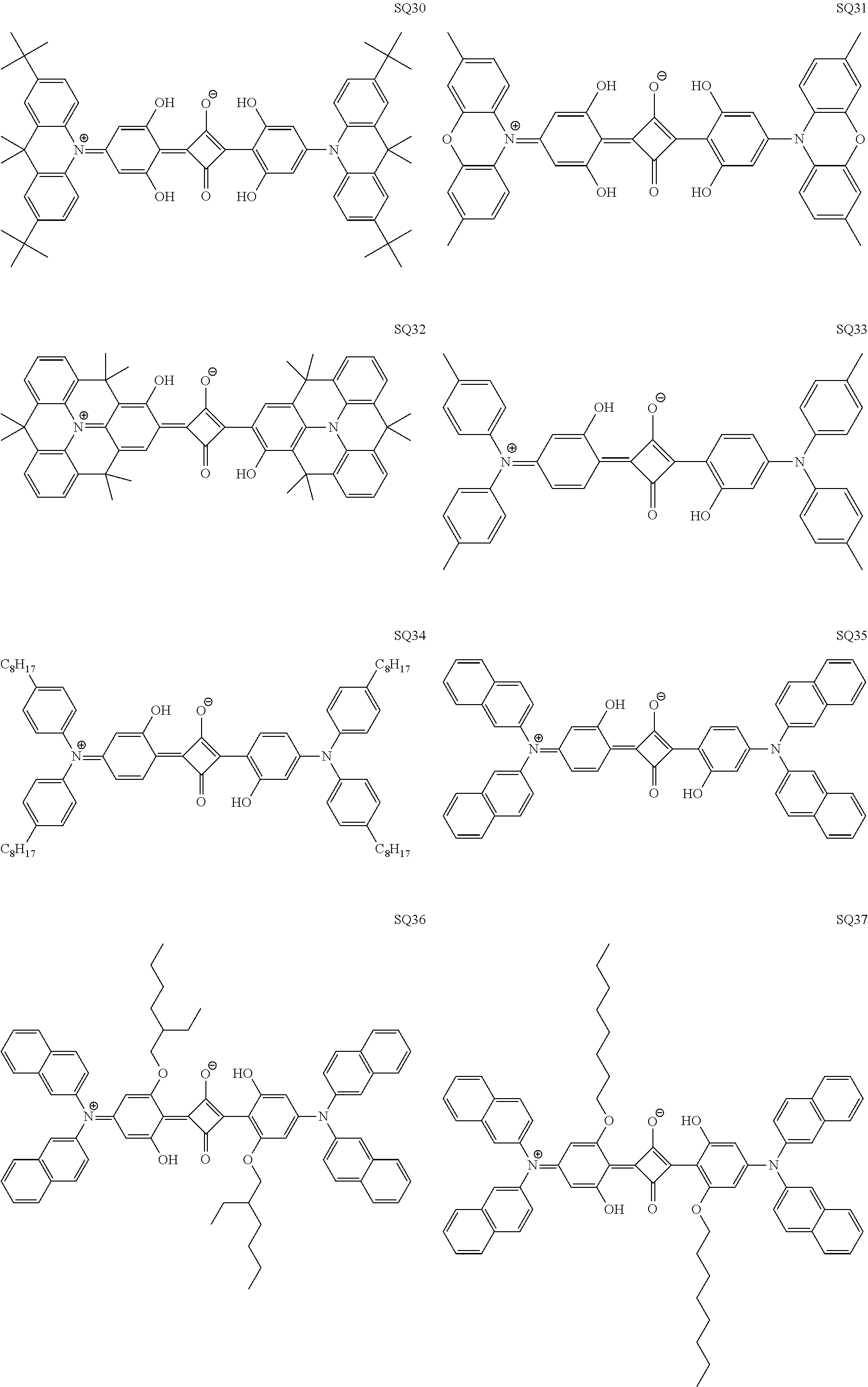

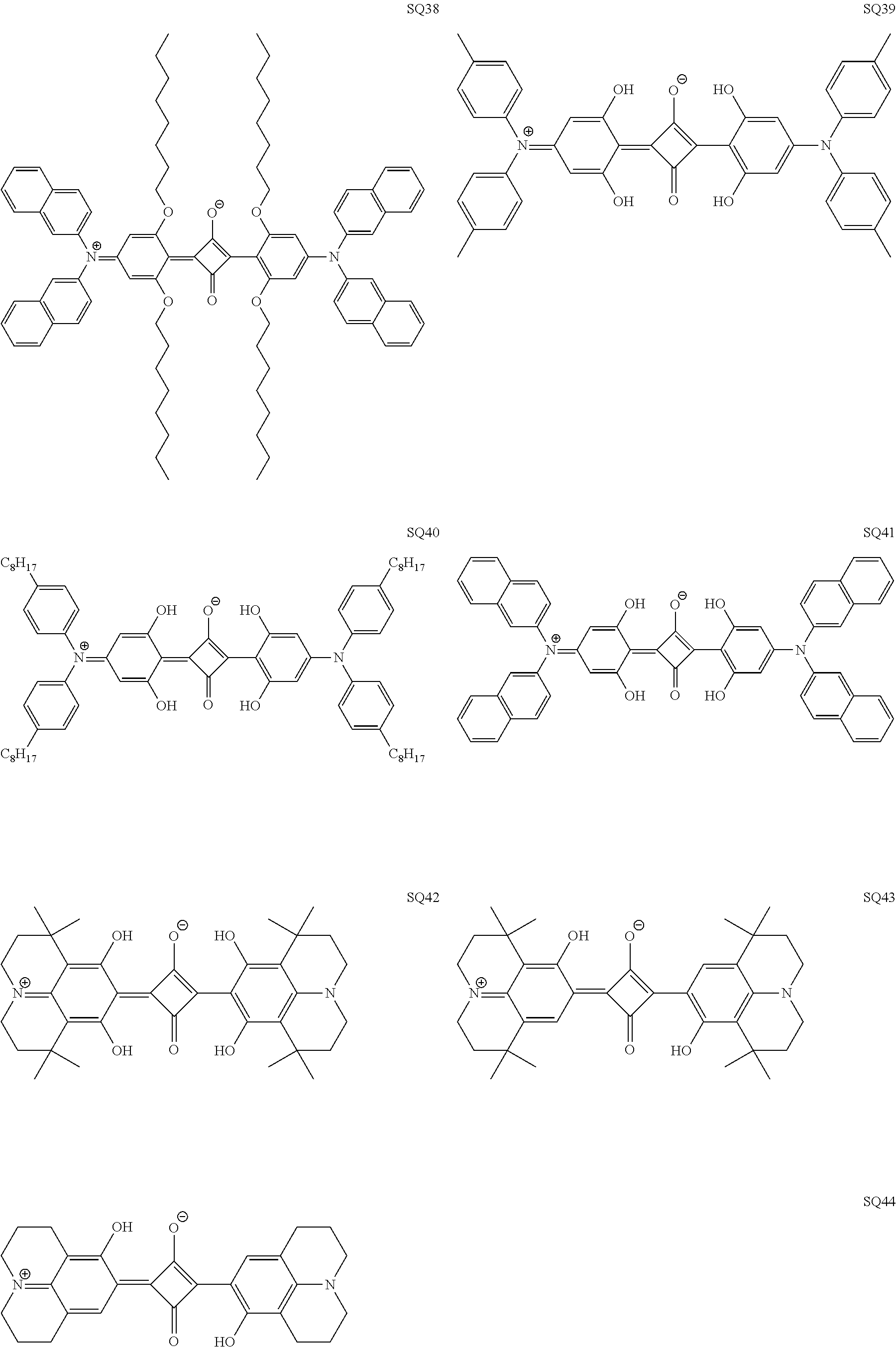

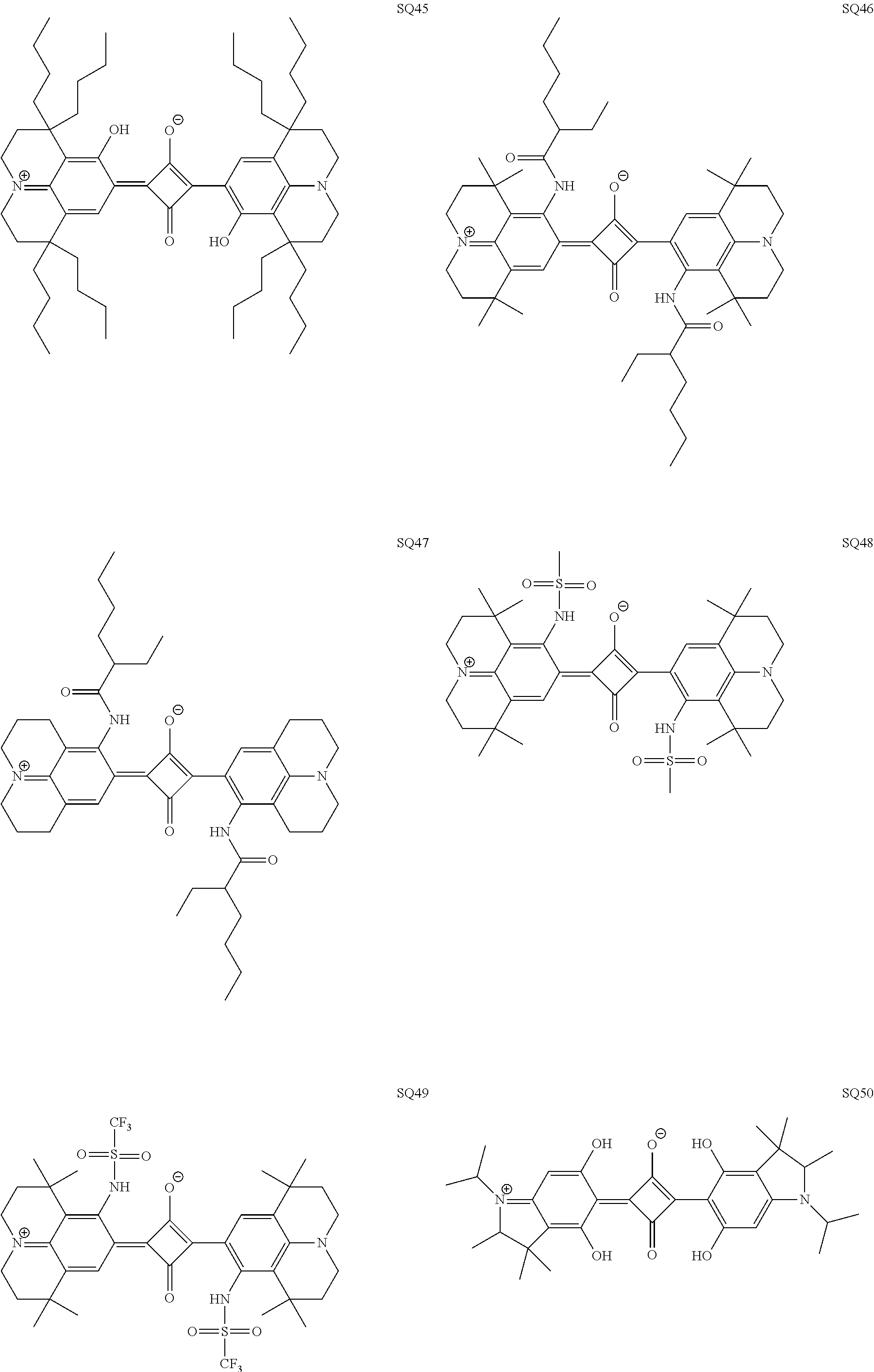

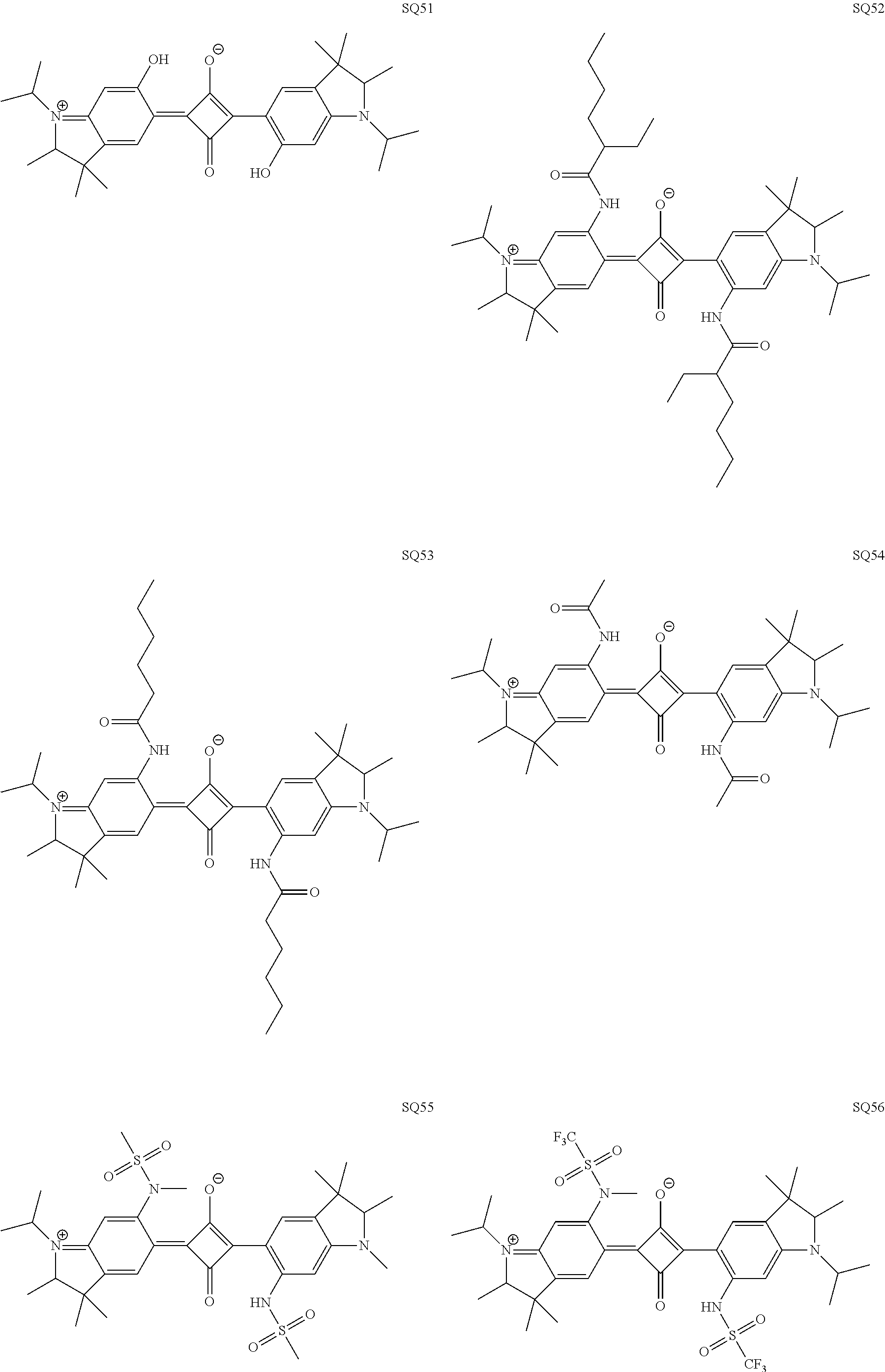

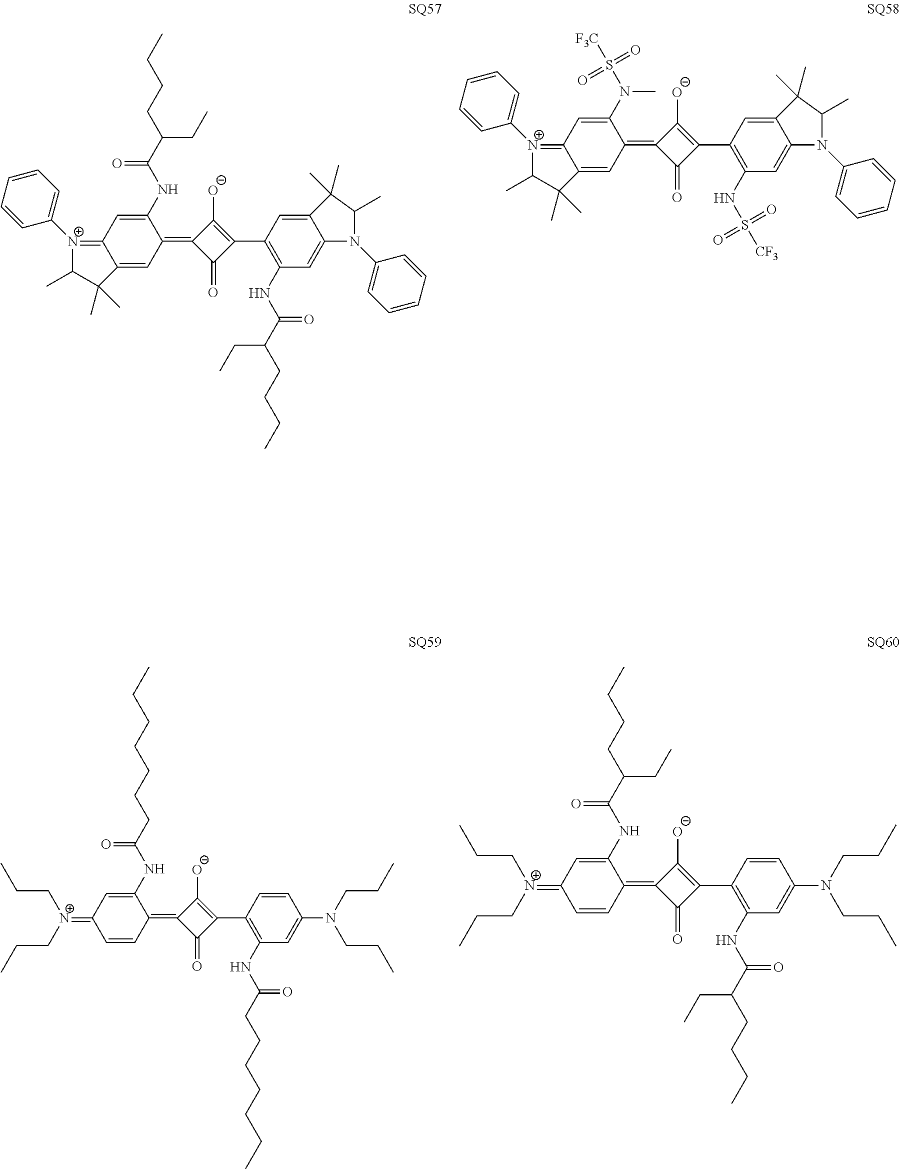 [SMILES: CCCCC(CC)C(=O)Nc1cc2c(cc1C1=C([O-])/C(=c3/cc4c(cc3NC(=O)C(CC)CCCC)=[N+](c3ccccc3)C(C)C4(C)C)C1=O)C(C)(C)C(C)N2c1ccccc1 ; CC1N(c2ccccc2)c2cc(NS(=O)(=O)C(F)(F)F)c(C3=C([O-])/C(=c4/cc5c(cc4N(C)S(=O)(=O)C(F)(F)F)=[N+](c4ccccc4)C(C)C5(C)C)C3=O)cc2C1(C)C ; CCCCCCCC(=O)NC1=CC(=[N+](CCC)CCC)C=C/C1=C1\C(=O)C(c2ccc(N(CCC)CCC)cc2NC(=O)CCCCCCC)=C1[O-] ; CCCCC(CC)C(=O)NC1=CC(=[N+](CCC)CCC)C=C/C1=C1\C(=O)C(c2ccc(N(CCC)CCC)cc2NC(=O)C(CC)CCCC)=C1[O-]]

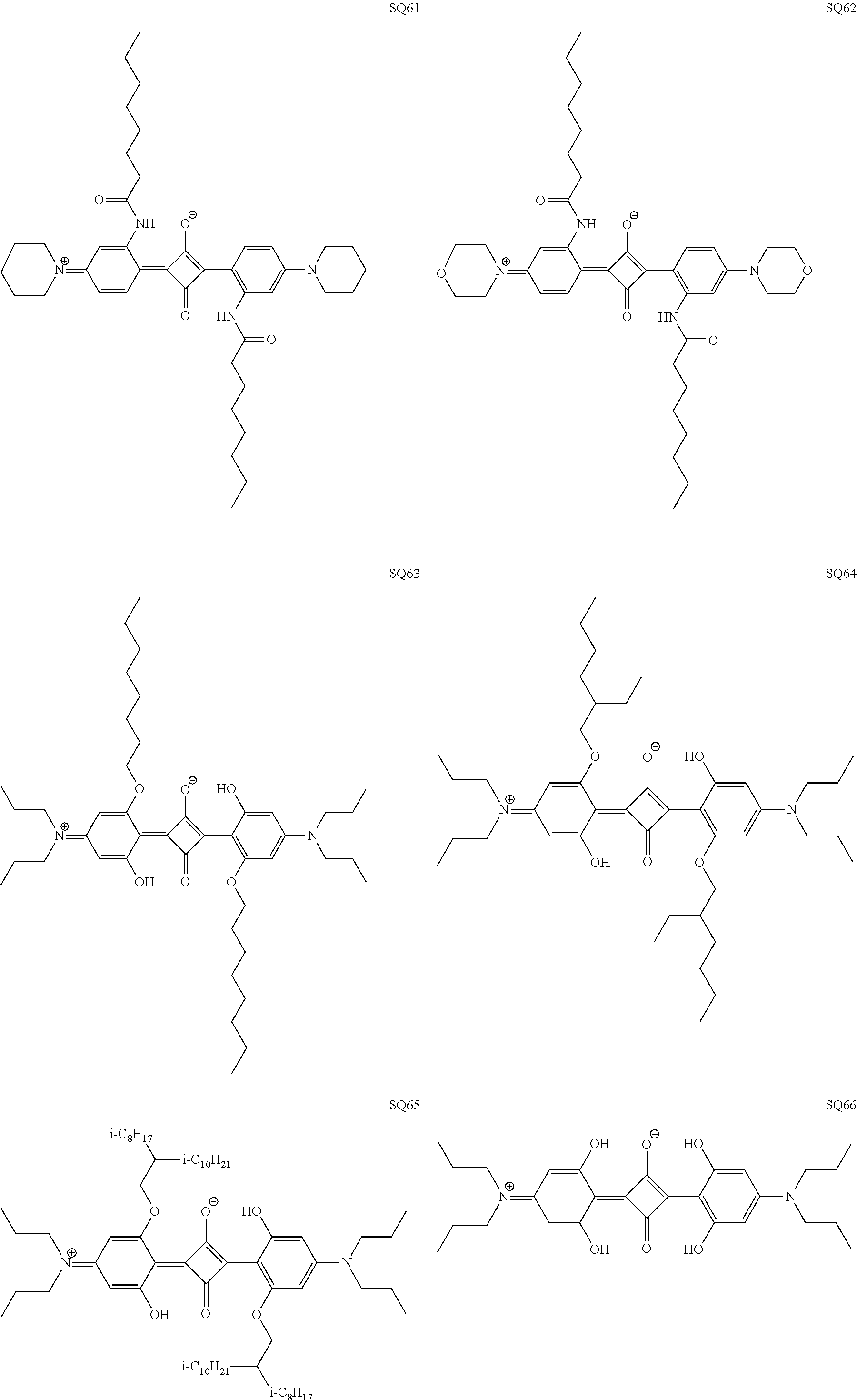

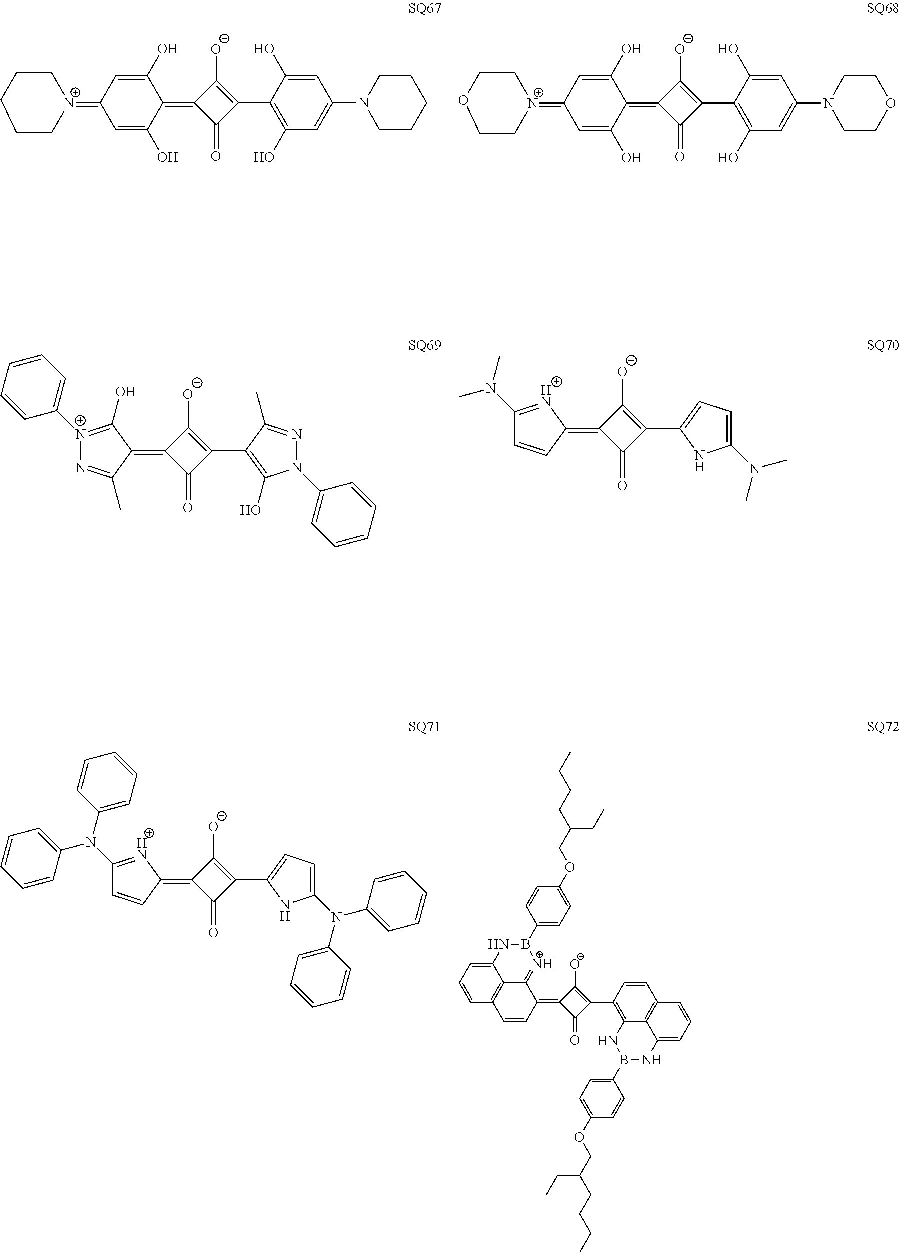

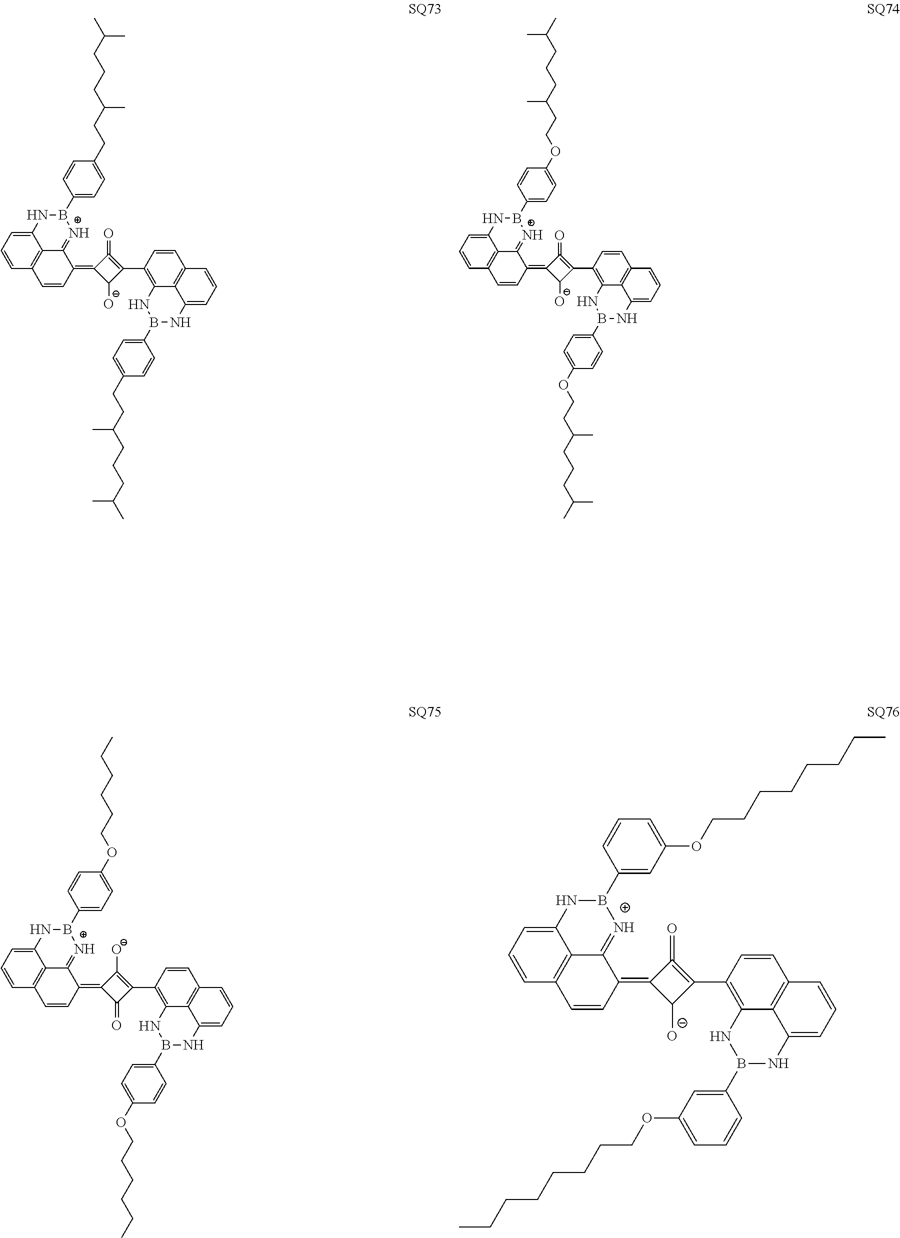

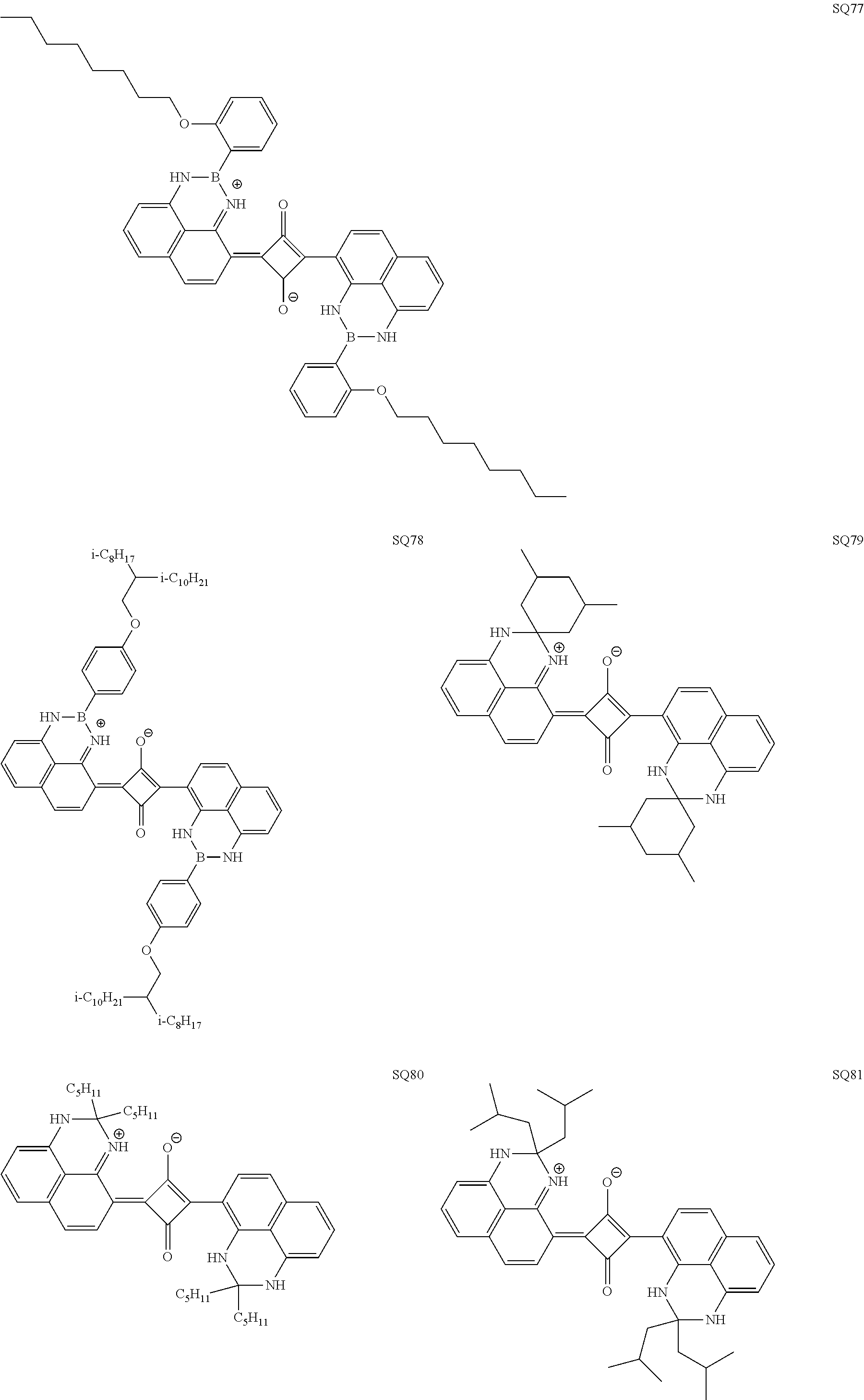

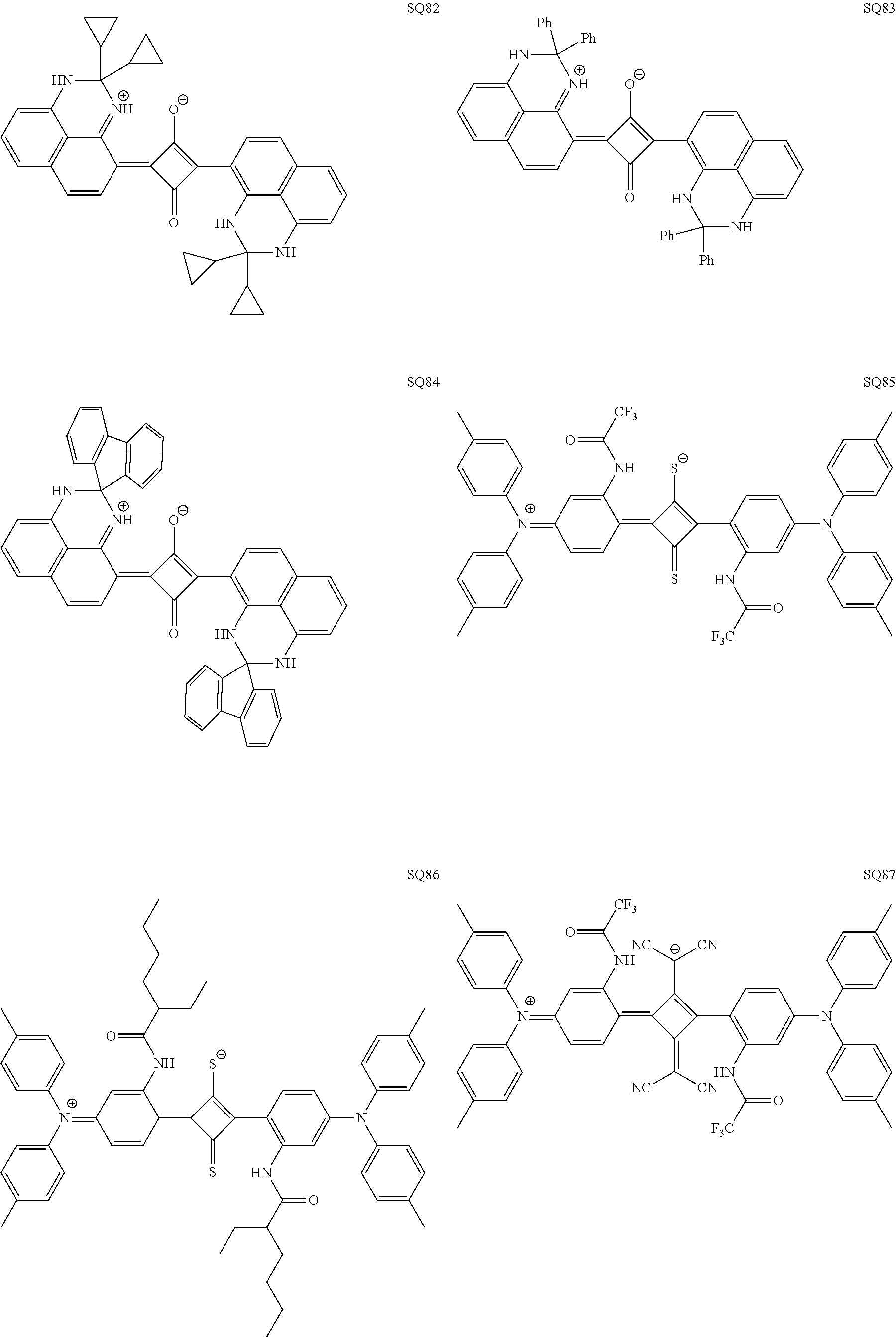

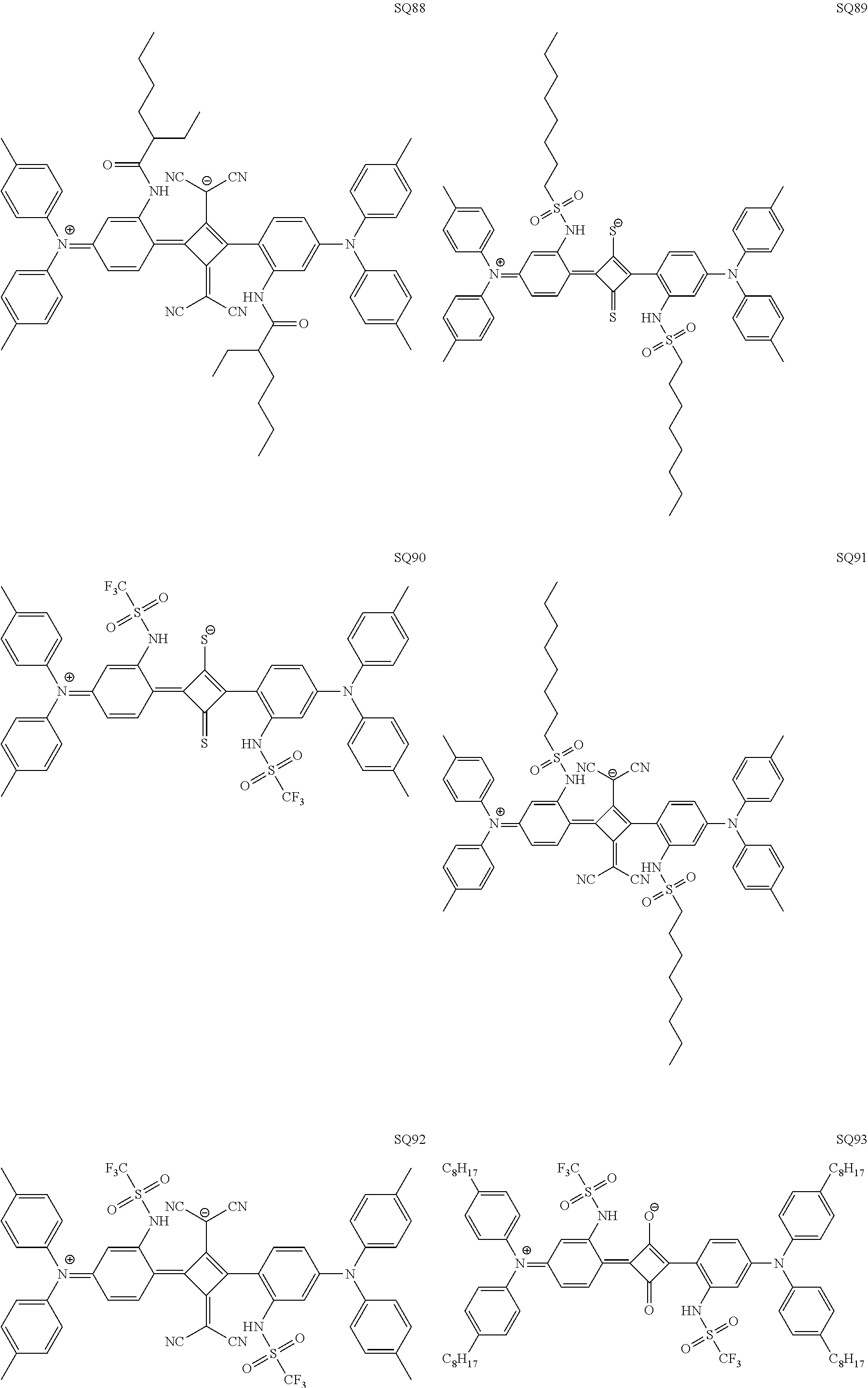

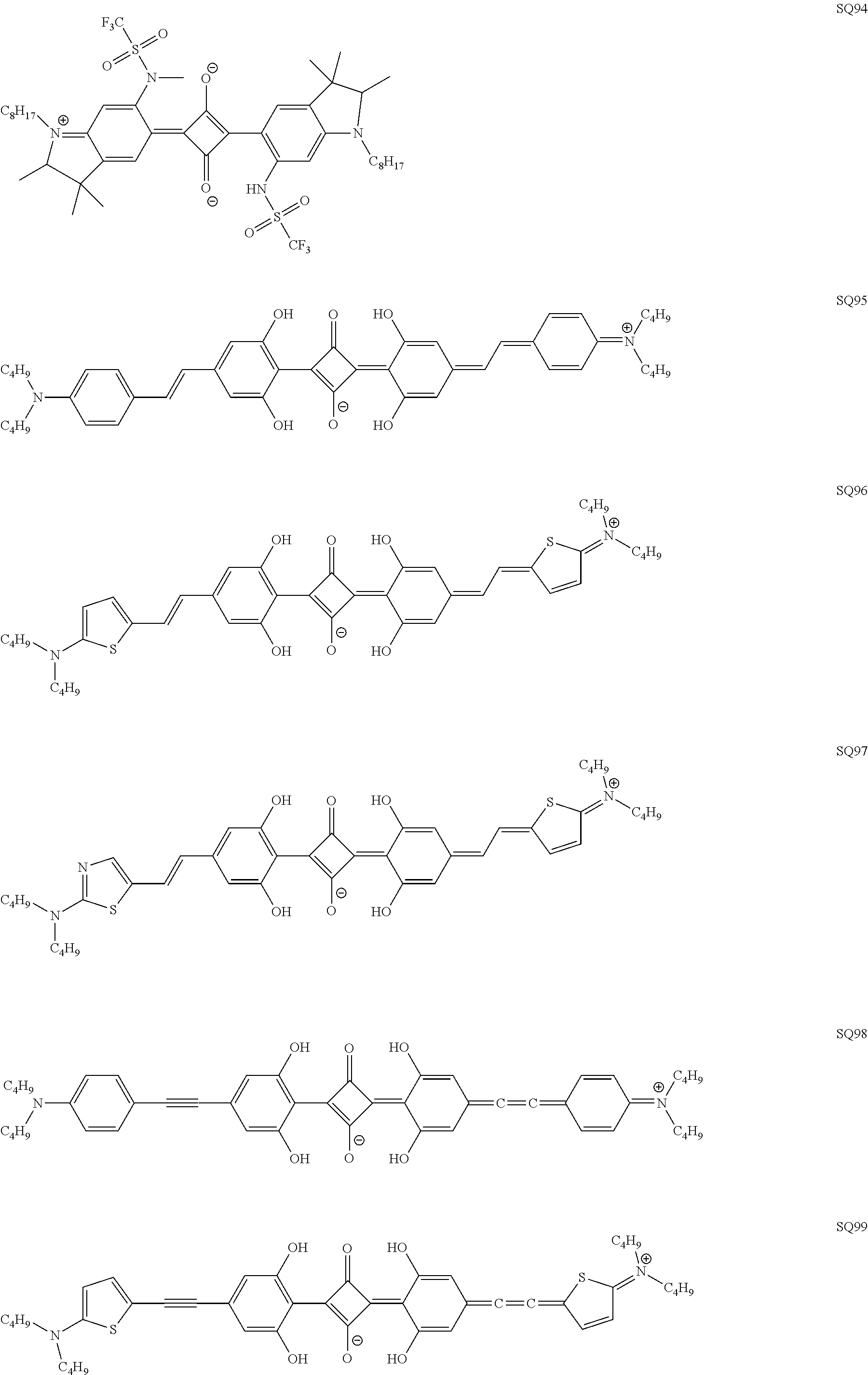

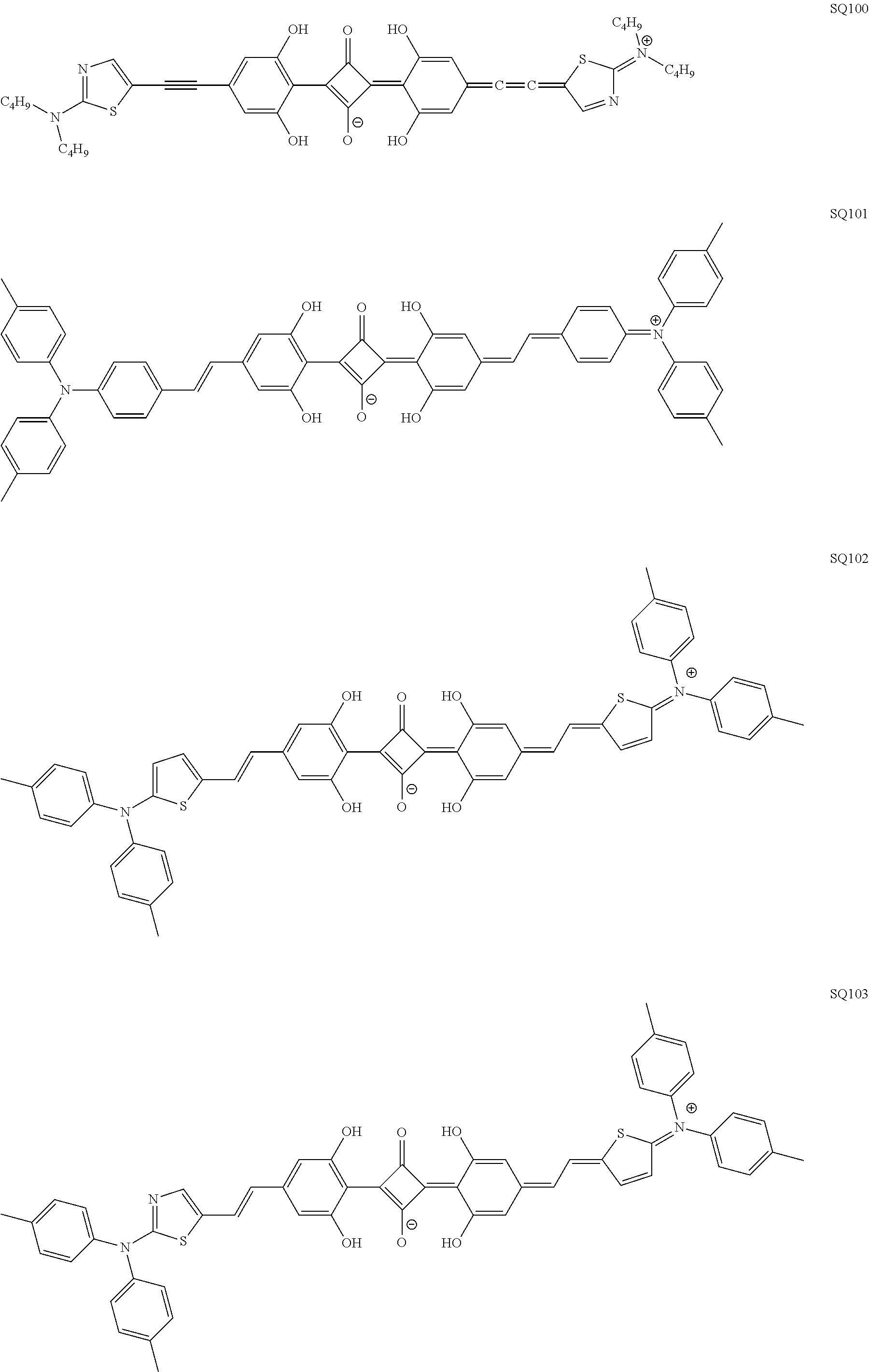

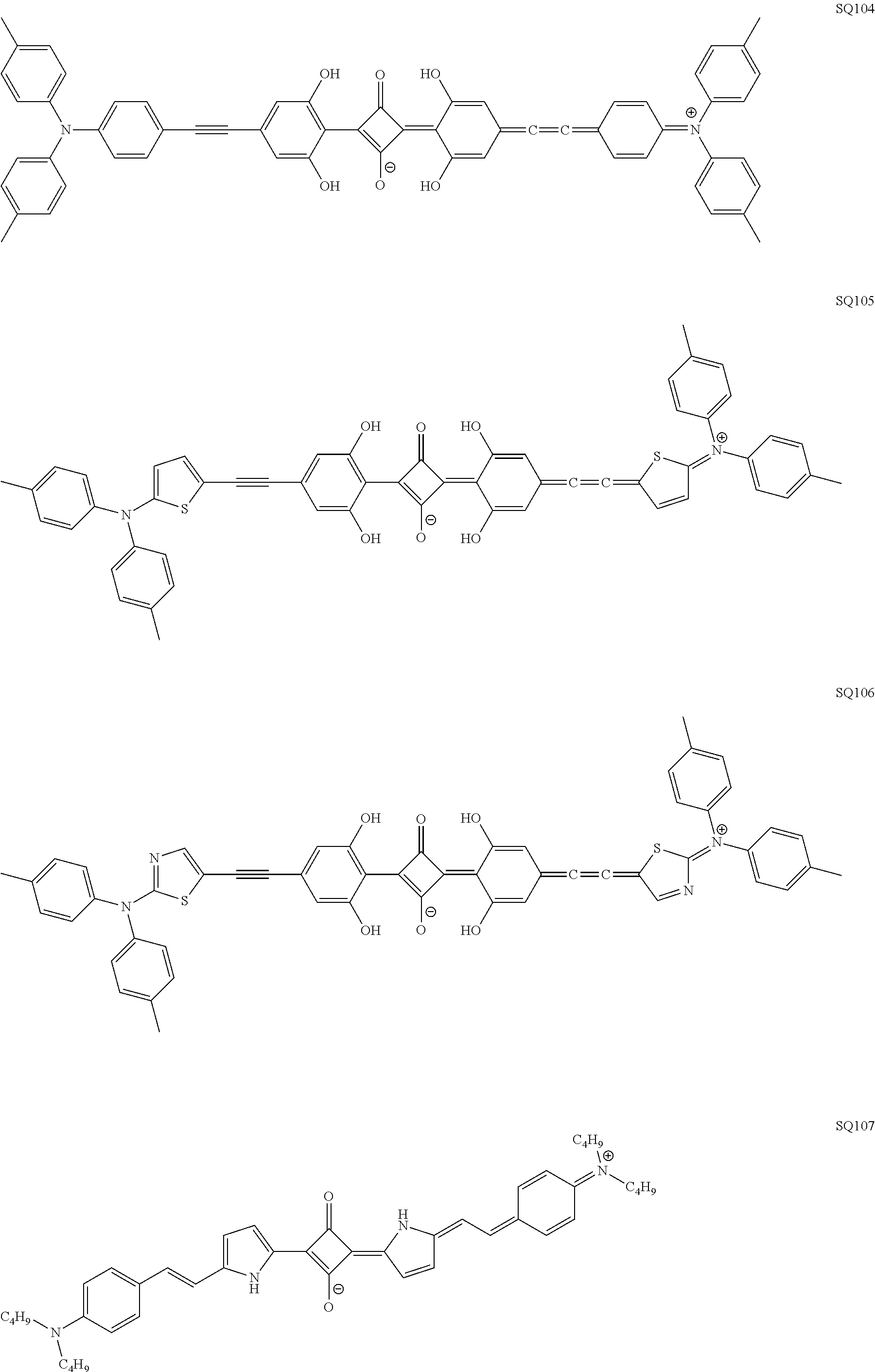

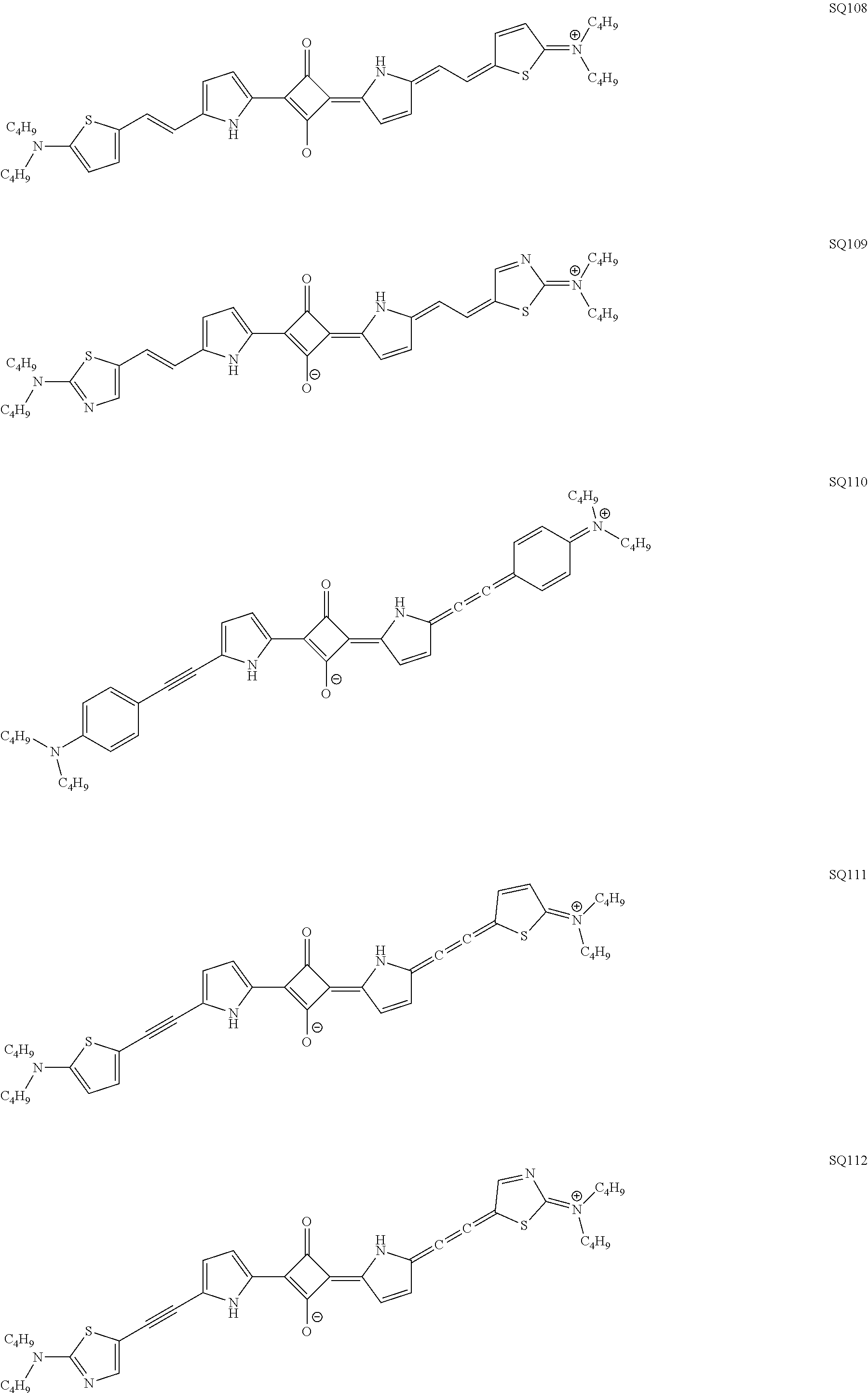

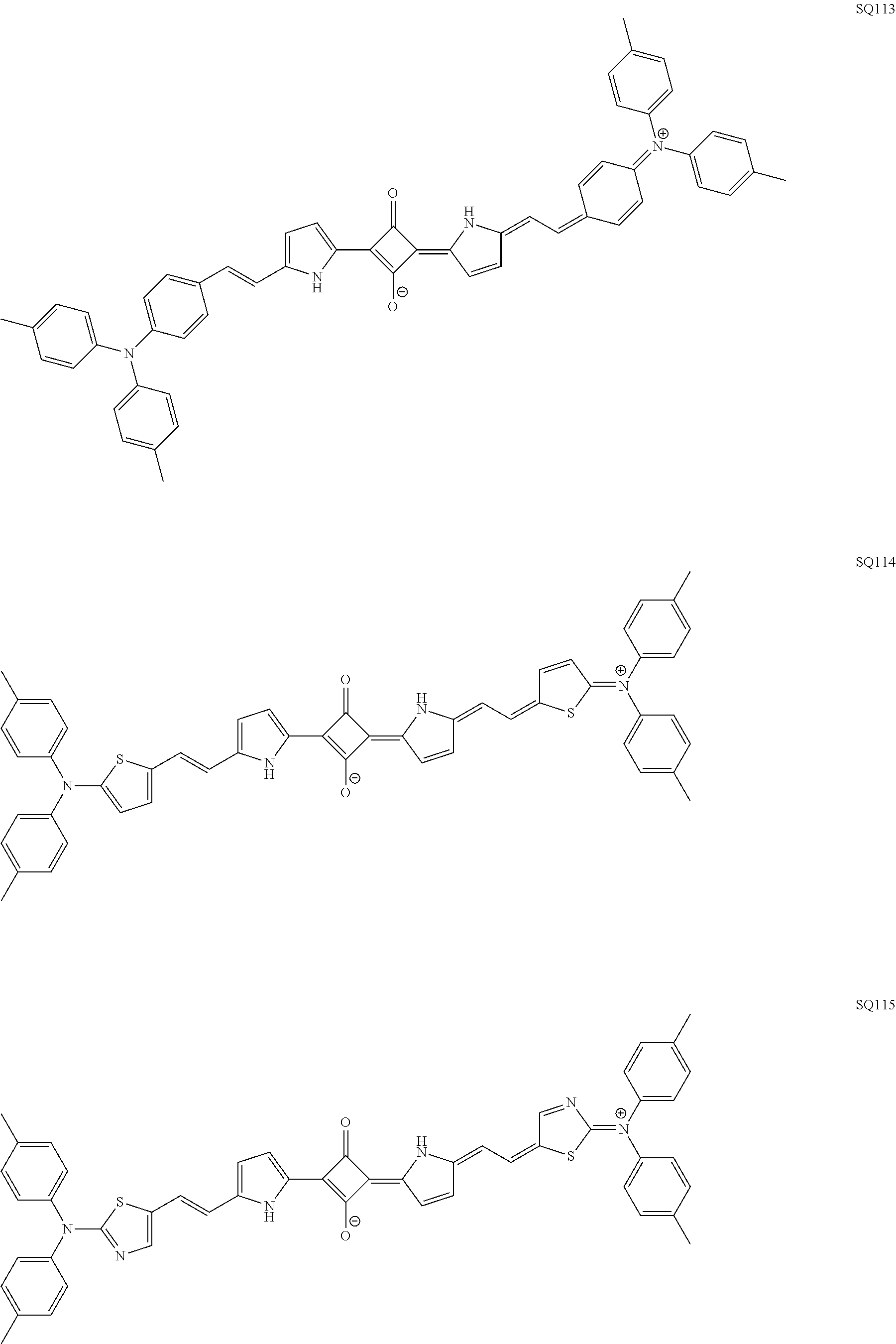

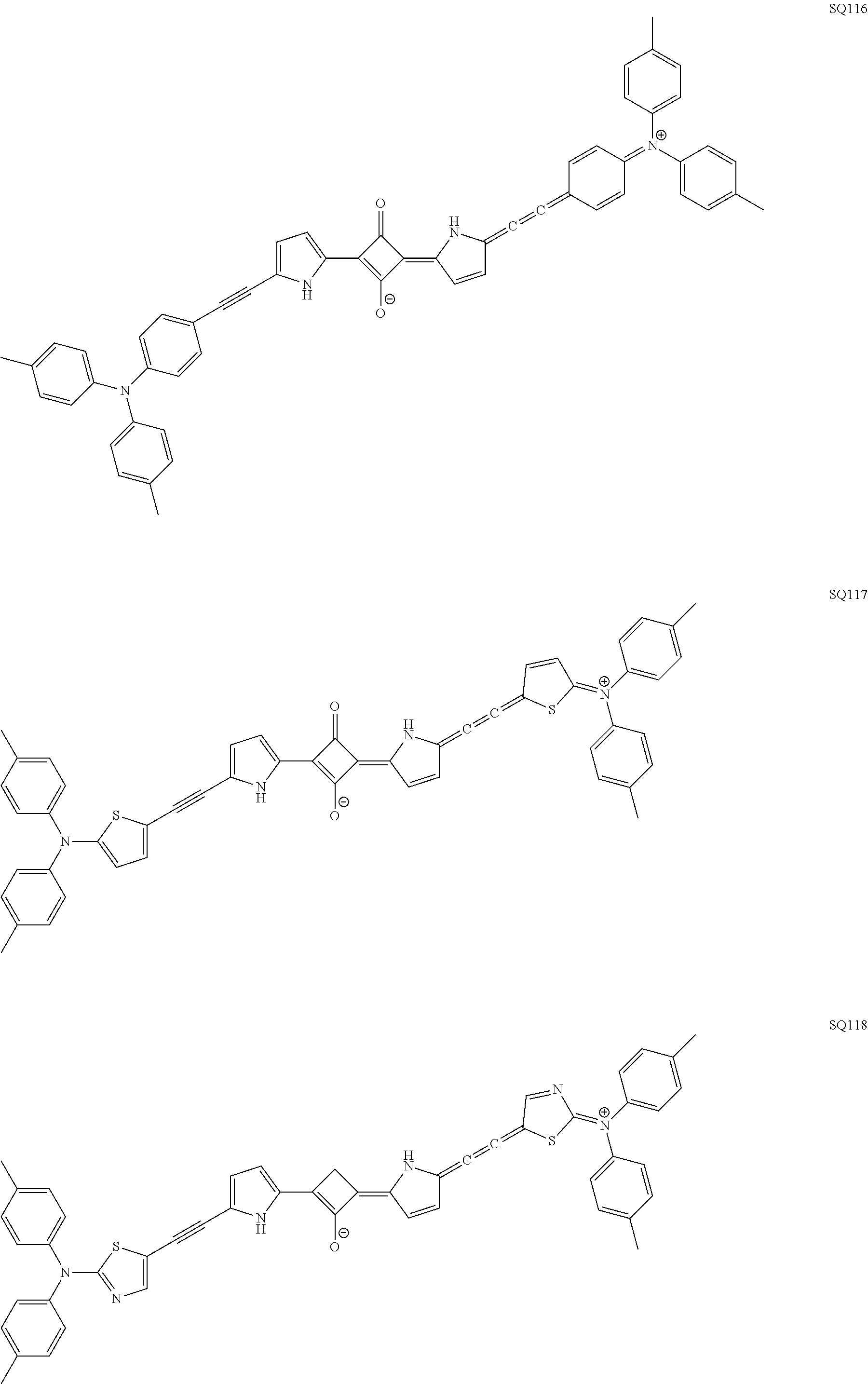

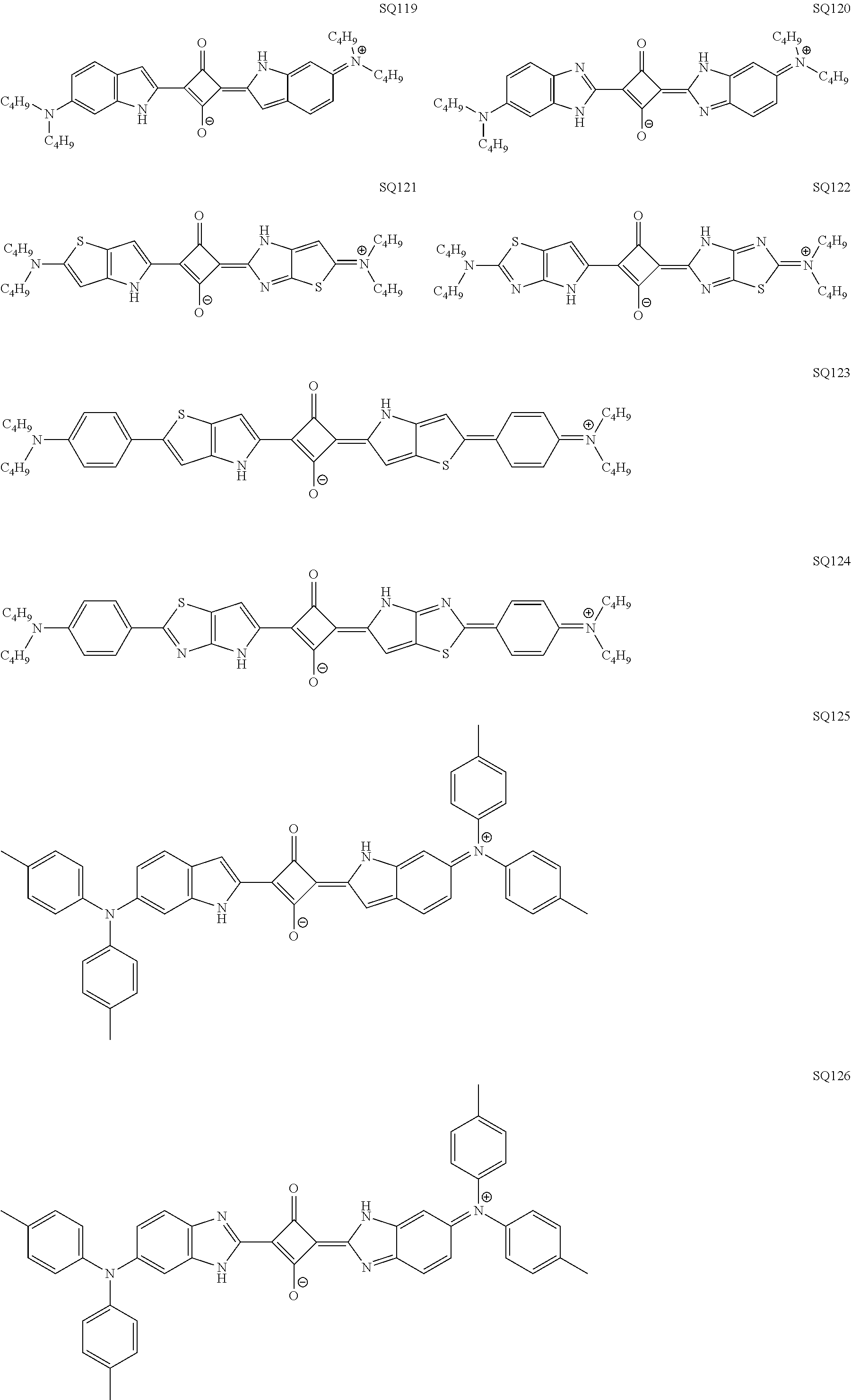

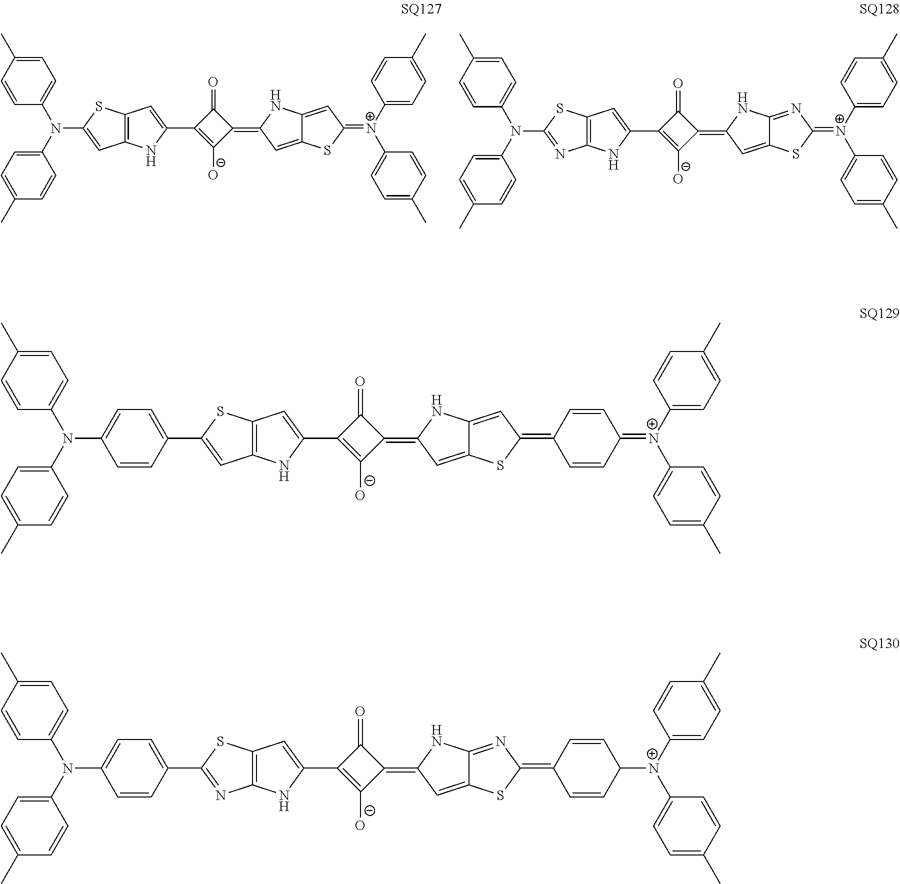

<<Other Near Infrared Absorbing Compounds>>

The composition according to the present invention may further include near infrared absorbing compounds (hereinafter, also referred to as “other near infrared absorbing compounds”) other than the squarylium compound (1). As the other near infrared absorbing compounds, a compound having an absorption maximum in a range of 700 to 1200 nm is preferable, and a compound having an absorption maximum in a range of 700 to 1000 nm is more preferable.

Examples of the other near infrared absorbing compounds include a phthalocyanine compound, a naphthalocyanine compound, a perylene compound, a pyrrolopyrrole compound, a cyanine compound, a dithiol metal complex compound, a naphthoquinone compound, an iminium compound, and an azo compound. Examples of the pyrrolopyrrole compound include a compound described in paragraphs “0016” to “0058” of JP2009-263614A. In addition, a squarylium compound other than the squarylium compound represented by Formula (1) may also be used. As the phthalocyanine compound, the naphthalocyanine compound, the iminium compound, the cyanine compound, the squarylium compound, or the croconium compound, for example, one of compounds described in paragraphs “0010” to “0081” of JP2010-111750A may be used, the content of which are incorporated in this specification. In addition the cyanine compound can be found in, for example, “Functional Colorants by Makoto Okawara, Masaru Matsuoka, Teijiro Kitao, and Tsuneoka Hirashima, published by Kodansha Scientific Ltd.”, the content of which is incorporated herein by reference.

In a case where the composition according to the present invention includes the other near infrared absorbing compounds, the content of the other near infrared absorbing compound is preferably 0.1 to 70 mass % with respect to the total solid content of the composition according to the present invention. The lower limit is preferably 0.5 mass % or higher and more preferably 1.0 mass % or higher. The upper limit is preferably 60 mass % or lower, and more preferably 50 mass % or lower. In a case where the composition according to the present invention includes two or more other near infrared absorbing compound, it is preferable that the total content of the two or more near infrared absorbing compounds is in the above-described range.

In addition, the composition according to the present invention may include substantially no other near infrared absorbing compounds. The composition according to the present invention including substantially no other near infrared absorbing compounds denotes that, for example, the content of the other near infrared absorbing compounds is 0.05 mass % or lower, 0.01 mass % or lower, or 0 mass % with respect to the total solid content of the composition according to the present invention.

<<Chromatic Colorant>>

The composition according to the present invention may include a chromatic colorant. In the present invention, “chromatic colorant” denotes a colorant other than a white colorant and a black colorant. It is preferable that the chromatic colorant is a colorant having an absorption maximum in a wavelength range of 400 to 650 nm.

In the present invention, the chromatic colorant may be a pigment or a dye. It is preferable that the chromatic colorant is a pigment.

The pigment is preferably an organic pigment, and examples thereof are as follows. However, the present invention is not limited to the examples:

Color Index (C.I.) Pigment Yellow 1, 2, 3, 4, 5, 6, 10, 11, 12, 13, 14, 15, 16, 17, 18, 20, 24, 31, 32, 34, 35, 35:1, 36, 36:1, 37, 37:1, 40, 42, 43, 53, 55, 60, 61, 62, 63, 65, 73, 74, 77, 81, 83, 86, 93, 94, 95, 97, 98, 100, 101, 104, 106, 108, 109, 110, 113, 114, 115, 116, 117, 118, 119, 120, 123, 125, 126, 127, 128, 129, 137, 138, 139, 147, 148, 150, 151, 152, 153, 154, 155, 156, 161, 162, 164, 166, 167, 168, 169, 170, 171, 172, 173, 174, 175, 176, 177, 179, 180, 181, 182, 185, 187, 188, 193, 194, 199, 213, and 214 (all of which are yellow pigments);

C.I. Pigment Orange 2, 5, 13, 16, 17:1, 31, 34, 36, 38, 43, 46, 48, 49, 51, 52, 55, 59, 60, 61, 62, 64, 71, and 73 (all of which are orange pigments);

C.I. Pigment Red 1, 2, 3, 4, 5, 6, 7, 9, 10, 14, 17, 22, 23, 31, 38, 41, 48:1, 48:2, 48:3, 48:4, 49, 49:1, 49:2, 52:1, 52:2, 53:1, 57:1, 60:1, 63:1, 66, 67, 81:1, 81:2, 81:3, 83, 88, 90, 105, 112, 119, 122, 123, 144, 146, 149, 150, 155, 166, 168, 169, 170, 171, 172, 175, 176, 177, 178, 179, 184, 185, 187, 188, 190, 200, 202, 206, 207, 208, 209, 210, 216, 220, 224, 226, 242, 246, 254, 255, 264, 270, 272, and 279 (all of which are red pigments);

C.I. Pigment Green 7, 10, 36, 37, 58, and 59 (all of which are green pigments); C.I. Pigment Violet 1, 19, 23, 27, 32, 37, and 42 (all of which are violet pigments); and

C.I. Pigment Blue 1, 2, 15, 15:1, 15:2, 15:3, 15:4, 15:6, 16, 22, 60, 64, 66, 79, and 80 (all of which are blue pigments).

Among these organic pigments, one kind may be used alone, or two or more kinds may be used in combination.

As the dye, well-known dyes can be used without any particular limitation. For example, a dye such as a pyrazole azo dye, an anilino azo dye, a triarylmethane dye, an anthraquinone dye, an anthrapyridone dye, a benzylidene dye, an oxonol dye, a pyrazolotriazole azo dye, a pyridone azo dye, a cyanine dye, a phenothiazine dye, a pyrrolopyrazole azomethine dye, a xanthene dye, a phthalocyanine dye, a benzopyran dye, an indigo dye, or a pyrromethene dye can be used. In addition, a polymer of the above-described dyes may be used. In addition, dyes described in JP2015-028144A and JP2015-34966A can also be used.

In addition, as the dye, an acid dye and/or a derivative thereof may be suitably used.

Furthermore, for example, a direct dye, a basic dye, a mordant dye, an acid mordant dye, an azoic dye, a dispersed dye, an oil-soluble dye, a food dye, and/or a derivative thereof can be suitably used.

Specific examples of the acid dye are shown below, but the present invention is not limited to these examples. For example, the following dyes and derivatives thereof can be used:
acid alizarin violet N;
acid blue 1, 7, 9, 15, 18, 23, 25, 27, 29, 40 to 45, 62, 70, 74, 80, 83, 86, 87, 90, 92, 103, 112, 113, 120, 129, 138, 147, 158, 171, 182, 192, 243, and 324:1;
acid chrome violet K;
acid Fuchsin and acid green 1, 3, 5, 9, 16, 25, 27, and 50;
acid orange 6, 7, 8, 10, 12, 50, 51, 52, 56, 63, 74, and 95;
acid red 1, 4, 8, 14, 17, 18, 26, 27, 29, 31, 34, 35, 37, 42, 44, 50, 51, 52, 57, 66, 73, 80, 87, 88, 91, 92, 94, 97, 103, 111, 114, 129, 133, 134, 138, 143, 145, 150, 151, 158, 176, 183, 198, 211, 215, 216, 217, 249, 252, 257, 260, 266, and 274;
acid violet 6B, 7, 9, 17, and 19;
acid yellow 1, 3, 7, 9, 11, 17, 23, 25, 29, 34, 36, 42, 54, 72, 73, 76, 79, 98, 99, 111, 112, 114, 116, 184, and 243; and
Food Yellow 3.

In addition to the above-described examples, an azo acid dye, a xanthene acid dye, and a phthalocyanine acid dye are preferably used, and acid dyes, such as C.I. Solvent Blue 44 and 38, C.I. Solvent Orange 45, Rhodamine B, and Rhodamine 110 and derivatives of the dyes are also preferably used.

Among these, it is preferable that the dye is a colorant selected from the group consisting of a triarylmethane dye, an anthraquinone dye, an azomethine dye, a benzylidene dye, an oxonol dye, a cyanine dye, a phenothiazine dye, a pyrrolopyrazole azo methine dye, a xanthene dye, a phthalocyanine dye, a benzopyran dye, an indigo dye, a pyrazole azo dye, an anilino azo dye, a pyrazolotriazole azo dye, a pyridone azo dye, an anthrapyridone dye, and a pyrromethene dye.

Further, a combination of a pigment and a dye may be used.

In a case where the composition according to the present invention includes a chromatic colorant, the content of the chromatic colorant is preferably 0.1 to 70 mass % with respect to the total solid content of the composition according to the present invention. The lower limit is preferably 0.5 mass % or higher and more preferably 1.0 mass % or higher. The upper limit is preferably 60 mass % or lower, and more preferably 50 mass % or lower.

The content of the chromatic colorant is preferably 10 to 1000 parts by mass and more preferably 50 to 800 parts by mass with respect to 100 parts by mass of the squarylium compound (1).

In addition, the total content of the chromatic colorant, the squarylium compound (1), and the other near infrared absorbing compounds is preferably 1 to 80 mass % with respect to the total solid content of the composition according to the present invention. The lower limit is preferably 5 mass % or higher and more preferably 10 mass % or higher. The upper limit is preferably 70 mass % or lower, and more preferably 60 mass % or lower.

In a case where the composition according to the present invention includes two or more chromatic colorants, it is preferable that the total content of the two or more chromatic colorants is in the above-described range.

<<Pigment Derivative>>

The composition according to the present invention may include a pigment derivative. Examples of the pigment derivative include a compound having a structure in which a portion of a pigment is substituted with an acidic group, or a basic group. It is preferable that the pigment derivative has an acidic group or a basic group from the viewpoints of dispersibility and dispersion stability.

<<Compound having Crosslinking Group (Crosslinking Compound)>>

The composition according to the present invention includes a compound having a crosslinking group (hereinafter, also referred to as “crosslinking compound”). By the composition according to the present invention including the crosslinking compound, a cured film having excellent heat resistance, light fastness, and solvent resistance can be manufactured.

In the present invention, the crosslinking compound denotes a compound which can form a crosslinked structure by causing crosslinking groups in the crosslinking compound to react with each other due to the action of a radical, an acid, heat, or the like and/or by causing a crosslinking group in the crosslinking compound to react with a functional group in a compound other than the crosslinking compound in the composition according to the present invention, the functional group (also referred to as “reactive group”) being reactive with the crosslinking group.

In the present invention, for example, the crosslinking compound may be in a chemical form of a monomer, a prepolymer, an oligomer, a polymer, or the like. Among these, a monomer is preferable. The details of the crosslinking compound can be found in paragraphs “0031” to “0202” of JP2013-253224A, the content of which is incorporated herein by reference.

In the present invention, as the crosslinking compound, a compound which has a group having an ethylenically unsaturated bond, a compound having a cyclic ether group, a compound having an alkoxysilyl group, a compound having a chlorosilyl group, a compound having an isocyanate group, or a carboxylic anhydride is preferable, and a compound which has a group having an ethylenically unsaturated bond, a compound having a cyclic ether group, a compound having an alkoxysilyl group, or a compound having a chlorosilyl group is more preferable. Examples of the group having an ethylenically unsaturated bond include a vinyl group, a styryl group, a (meth)allyl group, and a (meth)acryloyl group. Among these, a (meth)allyl group or a (meth)acryloyl group is preferable. Examples of the cyclic ether group include an epoxy group and an oxetanyl group. Among these, an epoxy group is preferable. Examples of the alkoxysilyl group include a monoalkoxysilyl group, a dialkoxysilyl group, and a trialkoxysilyl group. Among these, a dialkoxysilyl group or a trialkoxysilyl group, is preferable, and a trialkoxysilyl group is more preferable. Examples of the chlorosilyl group include a monochlorosilyl group, a dichlorosilyl group, and a trichlorosilyl group. Among these, a dichlorosilyl group or a trichlorosilyl group is preferable, and a trichlorosilyl group is more preferable.

In a case where a compound having an isocyanate group or a carboxylic anhydride is used as the crosslinking compound, it is preferable that the composition according to the present invention includes a crosslinking aid described below. The compound having an isocyanate group or the carboxylic anhydride can react with the crosslinking aid described below to form a strong crosslinked structure.

The content of the crosslinking compound is preferably 1 to 90 mass % with respect to the total solid content of the composition. The lower limit is preferably 2 mass % or higher, more preferably 5 mass % or higher, and still more preferably 10 mass % or higher. The upper limit is preferably 80 mass % or lower, and more preferably 75 mass % or lower.

In addition, the content of the crosslinking compound is preferably 1 to 1000 parts by mass with respect to 100 parts by mass of the squarylium compound (1). The lower limit is preferably 10 parts by mass or more and more preferably 50 parts by mass or more. The upper limit is preferably 500 parts by mass or less and more preferably 200 parts by mass or less.

As the crosslinking compound, one kind may be used alone, or two or more kinds may be used. In a case where two or more crosslinking compounds are used in combination, it is preferable that the total content of the two or more crosslinking compounds is in the above-described range.

(Compound which has Group Having Ethylenically Unsaturated Bond)

In the present invention, as the crosslinking compound, a compound which has a group having an ethylenically unsaturated bond can be used. It is preferable that the compound which has a group having an ethylenically unsaturated bond is a monomer. The molecular weight of the compound which has a group having an ethylenically unsaturated bond is preferably 100 to 3000. The upper limit is preferably 2000 or lower and more preferably 1500 or lower. The lower limit is preferably 150 or higher and more preferably 250 or higher. The compound which has a group having an ethylenically unsaturated bond is preferably a (meth)acrylate compound having 3 to 15 functional groups and more preferably a (meth)acrylate compound having 3 to 6 functional groups.

Examples of the compound can be found in paragraphs “0033” and “0034” of JP2013-253224A, the content of which is incorporated herein by reference. As the compound, ethyleneoxy-modified pentaerythritol tetraacrylate (as a commercially available product, NK ESTER ATM-35E manufactured by Shin-Nakamura Chemical Co., Ltd.), dipentaerythritol triacrylate (as a commercially available product, KAYARAD D-330 manufactured by Nippon Kayaku Co., Ltd.), dipentaerythritol tetraacrylate (as a commercially available product, KAYARAD D-320 manufactured by Nippon Kayaku Co., Ltd.), dipentaerythritol penta(meth)acrylate (as a commercially available product, KAYARAD D-310 manufactured by Nippon Kayaku Co., Ltd.), dipentaerythritol hexa(meth)acrylate (as a commercially available product, KAYARAD DPHA manufactured by Nippon Kayaku Co., Ltd., A-DPH-12E, manufactured by Shin-Nakamura Chemical Co., Ltd.), or a structure in which the (meth)acryloyl group is bonded through an ethylene glycol or a propylene glycol residue is preferable. In addition, oligomers of the above-described examples can be used. In addition, examples of the compound can be found in the description of a polymerizable compound in paragraphs “0034” to “0038” of JP2013-253224A, the content of which is incorporated herein by reference. Examples of the compound having an ethylenically unsaturated bond include a polymerizable monomer in paragraph “0477” of JP2012-208494A (corresponding to paragraph “0585” of US2012/0235099A), the content of which is incorporated herein by reference.

In addition, diglycerin ethylene oxide (EO)-modified (meth)acrylate (as a commercially available product, M-460 manufactured by Toagosei Co., Ltd.) is preferable. Pentaerythritol tetraacrylate (A-TMMT manufactured by Shin-Nakamura Chemical Co., Ltd.) or 1,6-hexanediol diacrylate (KAYARAD HDDA manufactured by Nippon Kayaku Co., Ltd.) is also preferable. Oligomers of the above-described examples can be used. For examples, RP-1040 (manufactured by Nippon Kayaku Co., Ltd.) is used.

The compound which has a group having an ethylenically unsaturated bond may have an acid group such as a carboxyl group, a sulfo group, or a phosphate group. Examples of the compound having an acid group include an ester of an aliphatic polyhydroxy compound and an unsaturated carboxylic acid. A compound having an acid group obtained by causing a nonaromatic carboxylic anhydride to react with an unreacted hydroxyl group of an aliphatic polyhydroxy compound is preferable. In particular, it is more preferable that, in this ester, the aliphatic polyhydroxy compound is pentaerythritol and/or dipentaerythritol. Examples of a commercially available product of the monomer having an acid group include M-305, M-510, and M-520 of ARONIX series as polybasic acid-modified acrylic oligomer (manufactured by Toagosei Co., Ltd.). The acid value of the compound having an acid group is preferably 0.1 to 40 mgKOH/g. The lower limit is preferably 5 mgKOH/g or higher. The upper limit is preferably 30 mgKOH/g or lower.

In addition, a compound having a caprolactone structure is also preferable as the compound which has a group having an ethylenically unsaturated bond. The compound having a caprolactone structure is not particularly limited as long as it has a caprolactone structure in the molecule thereof, and examples thereof include ϵ-caprolactone-modified polyfunctional (meth)acrylate obtained by esterification of a polyhydric alcohol, (meth)acrylic acid, and ϵ-caprolactone, the polyhydric alcohol being, for example, trimethylolethane, ditrimethylolethane, trimethylolpropane, ditrimethylolpropane, pentaerythritol, dipentaerythritol, tripentaerythritol, glycerin, diglycerol, or trimethylolmelamine. Examples of the compound having a caprolactone structure can be found in paragraphs “0042” to “0045” of JP2013-253224A, the content of which is incorporated herein by reference. Examples of the compound having a caprolactone structure include: DPCA-20, DPCA-30, DPCA-60, and DPCA-120 which are commercially available as KAYARADDPCA series manufactured by Nippon Kayaku Co., Ltd.; SR-494 (manufactured by Sartomer) which is a tetrafunctional acrylate having four ethyleneoxy chains; and TPA-330 (manufactured by Nippon Kayaku Co., Ltd.) which is a trifunctional acrylate having three isobutyleneoxy chains.

As the compound which has a group having an ethylenically unsaturated bond, a urethane acrylate described in JP1973-41708B (JP-S48-41708B), JP1976-37193A (JP-S51-37193A), JP1990-32293B (JP-H2-32293B), or JP1990-16765B (JP-H2-16765B), or a urethane compound having a ethylene oxide skeleton described in JP1983-49860B (JP-S58-49860B), JP1981-17654B (JP-S56-17654B), JP1987-39417B (JP-S62-39417B), or JP1987-39418B (JP-S62-39418B) is also preferable. In addition, a composition having an extremely high photospeed can be obtained by using an addition-polymerizable compound having an amino structure or a sulfide structure in the molecules described in JP1988-277653A (JP-S63-277653A), JP1988-260909A (JP-S63-260909A), or JP1989-105238A (JP-H1-105238A).

Examples of a commercially available product of the polymerizable compound include URETHANE OLIGOMER UAS-10 and UAB-140 (manufactured by Sanyo-Kokusaku Pulp Co., Ltd.), UA-7200 (manufactured by Shin-Nakamura Chemical Co., Ltd.), DPHA-40H (manufactured by Nippon Kayaku Co., Ltd.), and UA-306H, UA-306T, UA-3061, AH-600, T-600 and AI-600 (manufactured by Kyoeisha Chemical Co., Ltd.).

In the present invention, as the compound which has a group having an ethylenically unsaturated bond, a polymer which has a group having an ethylenically unsaturated bond at a side chain can be used. The content of a repeating unit which has a group having an ethylenically unsaturated bond at a side chain is preferably 5 to 100 mass % with respect to all the repeating units constituting the polymer. The lower limit is preferably 10 mass % or higher and more preferably 15 mass % or higher. The upper limit is preferably 90 mass % or lower, more preferably 80 mass % or lower, and still more preferably 70 mass % or lower.

The polymer may include other polymers in addition to the repeating unit which has a group having an ethylenically unsaturated bond at a side chain. The other repeating units may or may not have a functional group such as an acid group. Examples of the acid group include a carboxyl group, a sulfo group, and a phosphate group. As the acid group, one kind may be used, or two or more kinds may be used. The proportion of the repeating unit having an acid group is preferably 0 to 50 mass % with respect to all the repeating units constituting the polymer. The lower limit is preferably 1 mass % or higher and more preferably 3 mass % or higher. The upper limit is more preferably 35 mass % or lower, and still more preferably 30 mass % or lower.

Specific examples of the polymer include a copolymer including (meth)allyl (meth)acrylate and (meth)acrylic acid. Examples of a commercially available product of the polymerizable polymer include DIANAL NR series (manufactured by Mitsubishi Rayon Co., Ltd.), PHOTOMER 6173 (a COOH-containing polyurethane acrylic oligomer; manufactured by Diamond Shamrock Co., Ltd.), BISCOAT R-264 and KS Resist 106 (both of which are manufactured by Osaka Organic Chemical Industry Ltd.), CYCLOMER-P series (for example, ACA230AA) and PLAKCEL CF200 series (both of which manufactured by Daicel Corporation), EBECRYL 3800 (manufactured by Daicel-UCB Co., Ltd.), and ACRYCURE RD-F8 (manufactured by Nippon Shokubai Co., Ltd.).

(Compound Having Cyclic Ether Group)

In the present invention, as the crosslinking compound, a compound having a cyclic ether group can also be used. Examples of the cyclic ether group include an epoxy group and an oxetanyl group. Among these, an epoxy group is preferable.

Examples of the compound having a cyclic ether group include a polymer having a cyclic ether group at a side chain and a monomer or an oligomer having two or more cyclic ether groups in a molecule. Examples of the compound having a cyclic ether group include an epoxy resin which is a glycidyl-etherified product of a phenol compound, an epoxy resin which is a glycidyl-etherified product of various novolac resins, an alicyclic epoxy resin, an aliphatic epoxy resin, a heterocyclic epoxy resin, a glycidyl ester epoxy resin, a glycidyl amine epoxy resin, an epoxy resin which is a glycidylated product of a halogenated phenol, a condensate of a silicon compound having an epoxy group and another silicon compound, and a copolymer of a polymerizable unsaturated compound having an epoxy group and another polymerizable unsaturated compound.

As the compound having a cyclic ether group, an epoxy compound having a glycidyl group such as glycidyl (meth)acrylate or allyl glycidyl ether can also be used. For example, a monofunctional or polyfunctional glycidyl ether compound can also be used, and a polyfunctional aliphatic glycidyl compound is preferable.

It is also preferable that the compound having a cyclic ether group is a compound having an alicyclic epoxy group. Examples of the compound can be found in, for example, paragraph “0045” of JP2009-265518A, the content of which is incorporated herein by reference.

The compound having a cyclic ether group may include a polymer having an epoxy group or an oxetanyl group as a repeating unit.

The weight-average molecular weight of the compound having a cyclic ether group is preferably 500 to 5000000 and more preferably 1000 to 500000. As the compound, a commercially available product may be used, or a compound obtained by introducing an epoxy group into a side chain of the polymer may be used.

Examples of the epoxy resin which is a glycidyl-etherified product of a phenol compound include: 2-[4-(2,3-epoxypropoxy)phenyl]-2-[4-[1,1-bis[4-(2,3-hydroxy)phenyl]ethyl]phenyl]propane, bisphenol A, bisphenol F, bisphenol S, 4,4′-biphenol, tetramethyl bisphenol A, dimethyl bisphenol A, tetramethyl bisphenol F, dimethyl bisphenol F, tetramethyl bisphenol S, dimethyl bisphenol S, tetramethyl-4,4′-biphenol, dimethyl-4,4′-biphenol, 1-(4-hydroxyphenyl)-2-[4-(1,1-bis-(4-hydroxyphenyl)ethyl)phenyl]propane, 2,2′-methyl ene-bis(4-methyl-6-t-butylphenol), 4,4′-butylidene-bis(3-methyl-6-t-butylphenol), trishydroxyphenylmethane, resorcinol, hydroquinone, pyrogallol, phloroglucinol, a phenol having a diisopropylidene skeleton; a phenol having a fluorene skeleton such as 1,1-di-4-hydroxyphenyl fluorene; and an epoxy resin which is a glycidyl-etherified product of a polyphenol compound, such as phenolic polybutadiene.

Examples of the epoxy resin which is a glycidyl-etherified product of a novolac resin include glycidyl-etherified products of various novolac resins including: novolac resins which contain various phenols, for example, phenol, cresols, ethyl phenols, butyl phenols, octyl phenols, bisphenols such as bisphenol A, bisphenol F, or bisphenol S, or naphthols; phenol novolac resins having a xylylene skeleton; phenol novolac resins having a xylylene skeleton; phenol novolac resins having a biphenyl skeleton; or phenol novolac resins having a fluorene skeleton.

Examples of the alicyclic epoxy resin include an alicyclic epoxy resin having an aliphatic ring skeleton such as 3,4-epoxycyclohexylmethyl-(3,4-epoxy)cyclohexylcarboxylate or bis(3,4-epoxycyclohexylmethyl)adipate.

Examples of the aliphatic epoxy resin include glycidyl ethers of polyhydric alcohols such as 1,4-butanediol, 1,6-hexanediol, polyethylene glycol, or pentaerythritol.

Examples of the heterocyclic epoxy resin include an heterocyclic epoxy resin having a heterocycle such as an isocyanuric ring or a hydantoin ring.

Examples of the glycidyl ester epoxy resin include an epoxy resin including a carboxylic acid ester such as hexahydrophthalic acid diglycidyl ester.

Examples of the glycidyl amine epoxy resin include an epoxy resin which is a glycidylated product of an amine such as aniline or toluidine.

Examples of the epoxy resin which is a glycidylated product of a halogenated phenol include an epoxy resin which is a glycidylated product of a halogenated phenol such as brominated bisphenol A, brominated bisphenol F, brominated bisphenol S, brominated phenol novolac, brominated cresol novolac, chlorinated bisphenol S, or chlorinated bisphenol A.

Examples of a commercially available product of the copolymer of a polymerizable unsaturated compound having an epoxy group and another polymerizable unsaturated compound include MARPROOF G-0150M, G-0105SA, G-0130SP, G-0250SP, G-1005S, G-1005SA, G-1010S, G-2050M, G-01100, and G-01758. Examples of the polymerizable unsaturated compound having an epoxy group include glycidyl acrylate, glycidyl methacrylate, and 4-vinyl-1-cyclohexene-1,2-epoxide. In addition, examples of the other polymerizable unsaturated compound include methyl (meth)acrylate, benzyl (meth)acrylate, cyclohexyl (meth)acrylate, styrene, and vinyl cyclohexane. Among these, methyl (meth)acrylate, benzyl (meth)acrylate, or styrene is preferable.

The epoxy equivalent of the epoxy resin (compound having an epoxy group) is preferably 310 to 3300 g/eq, more preferably 310 to 1700 g/eq, and still more preferably 310 to 1000 g/eq. As the epoxy resin, one kind may be used alone, or a mixture of two or more kinds may be used.

Examples of a commercially available product of the compound having a cyclic ether group can be found in, for example, paragraph “0191” JP2012-155288A, the content of which is incorporated herein by reference.

In addition, for example, a polyfunctional aliphatic glycidyl ether compound such as DENACOL EX-212L, EX-214L, EX-216L, EX-321L, or EX-850L (all of which are manufactured by Nagase ChemteX Corporation) can be used. The above-described examples are low-chlorine products, but a commercially available product which is not a low-chlorine product such as EX-212, EX-214, EX-216, EX-321, or EX-850 can also be used.

Other examples include: ADEKA RESIN EP-4000S, EP-4003S, EP-4010S, and EP-4011S (all of which are manufactured by Adeka Corporation); NC-2000, NC-3000, NC-7300, XD-1000, EPPN-501, and EPPN-502 (all of which are manufactured by Adeka Corporation); JER1031S, CELLOXIDE 2021P, CELLOXIDE 2081, CELLOXIDE 2083, CELLOXIDE 2085, EHPE 3150, EPOLEAD PB 3600, and EPOLEAD PB 4700 (all of which are manufactured by Daicel Corporation); and CYCLOMER P ACA 200M, CYCLOMER P ACA 230AA, CYCLOMER P ACA Z250, CYCLOMER P ACA Z251, CYCLOMER P ACA Z300, and CYCLOMER P ACA Z320 (all of which are manufactured by Daicel Corporation).

Further, examples of a commercially available product of the phenol novolac epoxy resin include JER-157565, JER-152, JER-154, and JER-157570 (all of which are manufactured by Mitsubishi Chemical Corporation).

In addition, examples of a commercially available product of a polymer having an oxetanyl group at a side chain and a polymerizable monomer or an oligomer having two or more oxetanyl groups in a molecule include ARONE OXETANE OXT-121, OXT-221, OX-SQ, and PNOX (all of which are manufactured by Toagosei Co., Ltd.).

(Compound Having Alkoxysilyl Group, Compound Having Chlorosilyl Group)

In the present invention, as the crosslinking compound, a compound having an alkoxysilyl group or a compound having a chlorosilyl group can also be used. Specific examples include methyl trimethoxysilane, dimethyl dimethoxysilane, phenyl trimethoxysilane, methyltriethoxysilane, and dimethyl diethoxysilane, phenyltriethoxysilane, n-propyltrimethoxysilane, n-propyltriethoxysilane, hexyl trimethoxysilane, hexyl triethoxysilane, octyl triethoxysilane, decyl trimethoxysilane, 1,6-bis(trimethoxysilyl)hexane, trifluoropropyltrimethoxysilane, vinyl trimethoxysilane, vinyltriethoxysilane, 2-(3,4-epoxycyclohexyl)ethyltrimethoxysilane, 3-glycidoxypropylmethyldimethoxysilane, 3-glycidoxypropyltrimethoxysilane, 3-glycidoxypropylmethyldiethoxysilane, 3-glycidoxypropyltriethoxysilane, p-styryltrimethoxysilane, 3-methacryloxypropylmethyldimethoxysilane, 3-methacryloxypropyltrimethoxysilane, 3-methacryloxypropylethyldimethoxysilane, 3-methacryloxypropyltriethoxysilane, 3-acryloxypropyltrimethoxysilane, N-2-(aminoethyl)-3-aminopropylmethyldimethoxysilane, N-2-(aminoethyl)-3-aminopropyltrimethoxysilane, 3-aminopropyltrimethoxysilane, 3-aminopropyltriethoxysilane, 3-triethoxysilyl-N-(1,3-dimethyl-butylidene)propylamine, N-phenyl-3-aminopropyltrimethoxysilane, N-(vinylbenzyl)-2-aminoethyl-3-aminopropyltrimethoxysilane, tris-(trimethoxysilylpropyl)isocyanurate, 3-ureidopropyltriethoxysilane, 3-mercaptopropylmethyldimethoxysilane, 3-mercaptopropyltrimethoxysilane, and bis(triethoxysilylpropyl)tetrasulfide, 3-isocyanatepropyltriethoxysilane, methyltrichlorosilane, ethyltrichlorosilane, phenyltrichlorosilane, dichloro(methyl)phenylsilane, dimethyldichlorosilane, and diethyldichlorosilane.

Examples of a commercially available product of the silane coupling agent include KBM-13, KBM-22, KBM-103, KBE-13, KBE-22, KBE-103, KBM-3033, KBE-3033, KBM-3063, KBE-3063, KBE-3083, KBM-3103, KBM-3066, KBM-7103, SZ-31, KPN-3504, KBM-1003, KBE-1003, KBM-303, KBM-402, KBM-403, KBE-402, KBE-403, KBM-1403, KBM-502, KBM-503, KBE-502, KBE-503, KBM-5103, KBM-602, KBM-603, KBM-903, KBE-903, KBE-9103, KBM-573, KBM-575, KBM-9659, KBE-585, KBM-802, KBM-803, KBE-846, and KBE-9007 (all of which are manufactured by Shin-Etsu Chemical Co., Ltd.).

In addition, as the compound having an alkoxysilyl group or the compound having a chlorosilyl group, a polymer having an alkoxysilyl group or a chlorosilyl group at a side chain can also be used. For example, the following polymer can be used. In the following description, Me represents a methyl group.

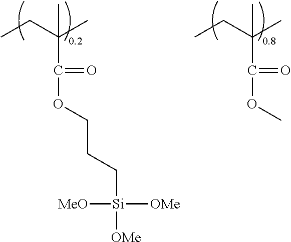

(Compound Having Isocyanate Group)

In the present invention, as the crosslinking compound, a compound having an isocyanate group can be used. As the compound having an isocyanate group, a compound having one or more isocyanate groups in one molecule is preferable, and a compound having two or more isocyanate groups in one molecule is preferable Examples of the compound having an isocyanate group include: an aromatic diisocyanate compound such as 2,4-tolylene diisocyanate, a dimer of 2,4-tolylene diisocyanate, 2,6-tolylene diisocyanate, p-xylylene diisocyanate, m-xylylene diisocyanate, 4,4′-diphenylmethane diisocyanate, 1,5-naphthylene diisocyanate, or 3,3′-dimethylbiphenyl-4,4′-diisocyanate; an aliphatic diisocyanate compound such as hexamethylene diisocyanate, trimethylhexamethylene diisocyanate, lysine diisocyanate, or dimer acid diisocyanate; an alicyclic diisocyanate compound such as isophorone diisocyanate, 4,4′-methylene bis(cyclohexyl isocyanate), methylcyclohexane-2,4 (or 2,6) diisocyanate, or 1,3-(isocyanatomethyl) cyclohexane; and a diisocyanate compound which is a reaction product between a diol and a diisocyanate such as an adduct of one mole of 1,3-butylene glycol and two moles of tolylene diisocyanate. In addition, an isocyanate described in paragraphs “0104” to “0106” and “0113” to “0120” of JP2013-253224A can also be used.

(Carboxylic Anhydride)

In the present invention, as the crosslinking compound, a carboxylic anhydride can be used. As the carboxylic anhydride, an aliphatic carboxylic anhydride or an aromatic carboxylic anhydride is preferable, and an aromatic carboxylic anhydride is more preferable. In addition, as the carboxylic anhydride, a tetracarboxylic dianhydride is preferable. Specific examples of the carboxylic anhydride include: pyromellitic dianhydride, 3,3′,4,4′-benzophenonetetracarboxylic dianhydride, 3,3′,4,4′-diphenyltetracarboxylic dianhydride, 2,3,6,7-naphthalenetetracarboxylic dianhydride, 1,4,5,8-naphthalenetetracarboxylic dianhydride, 4,4′-sulfonyldiphthalic dianhydride, 2,2-bis(3,4-dicarboxyphenyl)propane dianhydride, bis(3,4-dicarboxyphenyl)ether dianhydride, or 4,4′-[3,3′-(alkylphosphoryldiphenylene)-bis(iminocarbonyl)]diphthalic dianhydride; an aromatic tetracarboxylic dianhydride such as an adduct of hydroquinone diacetate and trimellitic anhydride or an adduct of diacetyldiamine and trimellitic anhydride; an alicyclic tetracarboxylic dianhydride such as 5-(2,5-dioxotetrahydrofuryl)-3-methyl-3-cyclohexene-1,2-dicarboxylic anhydride (EPICLON B-4400 manufactured by DIC Corporation), 1,2,3,4-cyclopentanetetracarboxylic dianhydride, 1,2,4,5-cyclohexanetetracarboxylic dianhydride, or tetrahydrofuran tetracarboxylic dianhydride; and an aliphatic tetracarboxylic dianhydride such as 1,2,3,4-butanetetracarboxylic dianhydride or 1,2,4,5-pentanetetracarboxylic dianhydride. The details of the carboxylic anhydride can be found in paragraphs “0166” to “0170” of JP2013-253224A, the content of which is incorporated herein by reference.

<<Photopolymerization Initiator>>

In a case where the compound which has a group having an ethylenically unsaturated bond is used as the crosslinking compound, it is preferable that the composition according to the present invention includes a photopolymerization initiator.

The photopolymerization initiator is not particularly limited as long as it has an ability to initiate the crosslinking reaction of the crosslinking compound, and can be selected from well-known photopolymerization initiators. For example, a photopolymerization initiator having photosensitivity to light in a range from the ultraviolet range to the visible range is preferable. it is preferable that the photopolymerization initiator is a photoradical polymerization initiator.

In addition, it is preferable that the photopolymerization initiator is at least one compound having a molar absorption coefficient of at least 50 in a range of about 300 nm to 800 nm (preferably 330 nm to 500 nm).

Examples of the photopolymerization initiator include: a halogenated hydrocarbon derivative (For example, a compound having a triazine skeleton or a compound having an oxadiazole skeleton); an acylphosphine compound such as acylphosphine oxide; an oxime compound such as hexaarylbiimidazole or an oxime derivative; an organic peroxide, a thio compound, a ketone compound, an aromatic onium salt, keto oxime ether, an aminoacetophenone compound, and hydroxyacetophenone. Examples of the halogenated hydrocarbon compound having a triazine skeleton include a compound described in Bull. Chem. Soc. Japan, 42, 2924 (1969) by Wakabayshi et al., a compound described in Great Britain Patent No. 1388492, a compound described in JP1978-133428A (JP-553-133428A), a compound described in Great German Patent No. 3337024, a compound described in J. Org. Chem.; 29, 1527 (1964) by F. C. Schaefer et al., a compound described in JP1987-58241A (JP-562-58241A), a compound described in JP1993-281728A (JP-H5-281728A), a compound described in JP1993-34920A (JP-55-34920A), and a compound described in U.S. Pat. No. 4,212,976A (for example, a compound having an oxadiazole skeleton).

In addition, from the viewpoint of exposure sensitivity, a compound selected from the group consisting of a trihalomethyltriazine compound, a benzyldimethylketanol compound, an α-hydroxy ketone compound, an α-aminoketone compound, an acylphosphine compound, a metallocene compound, an oxime compound, a triarylimidazole dimer, an onium compound, a benzothiazole compound, a benzophenone compound, an acetophenone compound and a derivative thereof, a cyclopentadiene-benzene-iron complex and a salt thereof, and a halomethyl oxadiazole compound, a 3-aryl-substituted coumarin compound is preferable.

Among these, at least one compound selected from the group consisting of a trihalomethyltriazine compound, an α-aminoketone compound, an acylphosphine compound, an oxime compound, a triarylimidazole dimer, an onium compound, a benzophenone compound, and an acetophenone compound is preferable, and at least one compound selected from the group consisting of a trihalomethyltriazine compound, an α-aminoketone compound, an oxime compound, a triarylimidazole dimer, and a benzophenone compound is more preferable.

In particular, in a case where the cured film according to the present invention is used for a solid image pickup element, it is necessary to form a fine pattern in a sharp shape, and thus it is important to obtain excellent curing properties and perform development without a residue remaining in a non-exposed portion. From these viewpoint, it is more preferable that an oxime compound is used as the photopolymerization initiator. In particular, in a case where a fine pattern is formed in a solid image pickup element, a stepper is used for exposure for curing, and this exposure device may be damaged by halogen, and it is also necessary to reduce the addition amount of the photopolymerization initiator to be small. In consideration of this point, it is more preferable an oxime compound is used as the photopolymerization initiator for forming a fine pattern in a solid image pickup element or the like. Specific examples of the photopolymerization initiator can be found in paragraphs “0265” to “0268” of JP2013-29760A, the content of which is incorporated herein by reference.

As the photopolymerization initiator, an α-hydroxyketone compound (hydroxyacetophenone compound), an α-aminoketone compound (aminoacetophenone compound), or an acylphosphine compound can also be preferably used. More specifically, for example, an aminoacetophenone initiator described in JP1998-291969A (JP-H10-291969A) or an acylphosphine initiator described in JP4225898B can also be used.

As the α-hydroxyketone compound, for example, IRGACURE-184, DAROCUR-1173, IRGACURE-500, IRGACURE-2959, or IRGACURE-127 (trade name, all of which are manufactured by BASF SE) can be used.

As the α-aminoketone compound, IRGACURE-907, IRGACURE-369, or IRGACURE-379EG (trade name, all of which are manufactured by BASF SE) which is a commercially available product can be used. As the α-aminoketone compound, a compound described in JP2009-191179A whose absorption wavelength is adjusted to match with that of a light source having a long wavelength of, for example, 365 nm or 405 nm can also be used.

As the acylphosphine compound, IRGACURE-819, or DAROCUR-TPO (trade name, all of which are manufactured by BASF SE) which is a commercially available product can be used.

As the photopolymerization initiator, for example, an oxime compound is more preferable.

Specific examples of the oxime compound include a compound described in JP2001-233842A, a compound described in JP2000-80068A, and a compound described in JP2006-342166A.

Examples of the oxime compound which can be preferably used in the present invention include 3-benzoyloxyiminobutane-2-one, 3-acetoxyiminobutane-2-one, 3-propionyloxyiminobutane-2-one, 2-acetoxyiminopentane-3-one, 2-acetoxyimino-1-phenylpropane-1-one, 2-benzoyloxyimino-1-phenylpropane-1-one, 3-(4-toluene sulfonyloxy)iminobutane-2-one, and 2-ethoxycarbonyloxyimino-1-phenylpropane-1-one.

In addition, examples of the oxime compound include a compound described in J.C.S. Perkin II (1979), pp. 1653-1660, J.C.S. Perkin II (1979), pp. 156-162 and Journal of Photopolymer Science and Technology (1995), pp. 202-232, JP2000-66385A, JP2000-80068A, JP2004-534797A, or JP2006-342166A. As a commercially available product of the oxime compound, IRGACURE-OXE01, IRGACURE-OXE02, IRGACURE-OXE03, or IRGACURE-OXE04 (all of which are manufactured by BASF SE) can also be preferably used. In addition, TR-PBG-304 (manufactured by Changzhou Tronly New Electronic Materials Co., Ltd.), ADEKA ARKLS NCI-930 (manufactured by Adeka Corporation), ADEKA OPTOMER N-1919 (manufactured by Adeka Corporation, a photopolymerization initiator 2 described in JP2012-14052A) can also be used.

In addition, in addition to the above-described oxime compounds, for example, a compound described in JP2009-519904A in which oxime is linked to a N-position of a carbazole ring, a compound described in U.S. Pat. No. 7,626,957B in which a hetero substituent is introduced into the benzophenone site, a compound described in JP2010-15025A or US2009/292039A in which a nitro group is introduced into a colorant site, a ketoxime compound described in WO2009/131189A, a compound described in U.S. Pat. No. 7,556,910B having a triazine skeleton and an oxime skeleton in the same molecule, a compound described in JP2009-221114A having an absorption maximum at 405 nm and having excellent sensitivity to a light source of g-rays, or a compound described in paragraphs “0076” to “0079” of JP2014-137466A may be used.

Other preferable examples of the oxime compound can be found in paragraphs “0274” to “0275” of JP2013-29760A, the content of which is incorporated herein by reference.

Specifically, as the oxime compound, a compound represented by the following Formula (OX-1) is preferable. In the oxime compound, an N—O bond of oxime may form an (E) isomer, a (Z) isomer, or a mixture of an (E) isomer and a (Z) isomer.

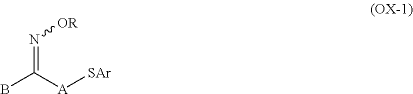

In Formula (OX-1), R and B each independently represent a monovalent substituent, A represents a divalent organic group, and Ar represents an aryl group.

In Formula (OX-1), it is preferable that the monovalent substituent represented by R is a monovalent non-metal atomic group.

Examples of the monovalent non-metal atomic group include an alkyl group, an aryl group, an acyl group, an alkoxycarbonyl group, an aryloxycarbonyl group, a heterocyclic group, an alkylthiocarbonyl group, and an arylthiocarbonyl group. In addition, these groups may have one or more substituents. In addition, the above-described substituent may have another substituent.

Examples of the substituent include a halogen atom, an aryloxy group, an alkoxycarbonyl group or aryloxycarbonyl group, an acyloxy group, an acyl group, an alkyl group, and an aryl group.

In Formula (OX-1), as the monovalent substituent represented by B, an aryl group, a heterocyclic group, an arylcarbonyl group, or a heterocyclic carbonyl group is preferable. These groups may have one or more substituents. Examples of the substituent are as described above.

In Formula (OX-1), as the divalent organic group represented by A, an alkylene group having 1 to 12 carbon atoms, a cycloalkylene group, or an alkynylene group is preferable. These groups may have one or more substituents. Examples of the substituent are as described above.

The oxime compound preferably has an absorption maximum in a wavelength range of 350 nm to 500 nm, more preferably has an absorption wavelength in a wavelength range of 360 nm to 480 nm, and still more preferably has a high absorbance at 365 nm and 405 nm.

The molar absorption coefficient of the oxime compound at 365 nm or 405 nm is preferably 1000 to 300000, more preferably 2000 to 300000, and still more preferably 5000 to 200000 from the viewpoint of sensitivity.

The molar absorption coefficient of the compound can be measured using a well-known method. For example, it is preferable that the absorption coefficient can be measured using an ultraviolet-visible spectrophotometer (Cary-5 spectrophotometer, manufactured by Varian Medical Systems, Inc.) and ethyl acetate as a solvent at a concentration of 0.01 g/L.

Hereinafter, specific examples of the oxime compound which are preferably used in the present invention are shown below, but the present invention is not limited thereto.

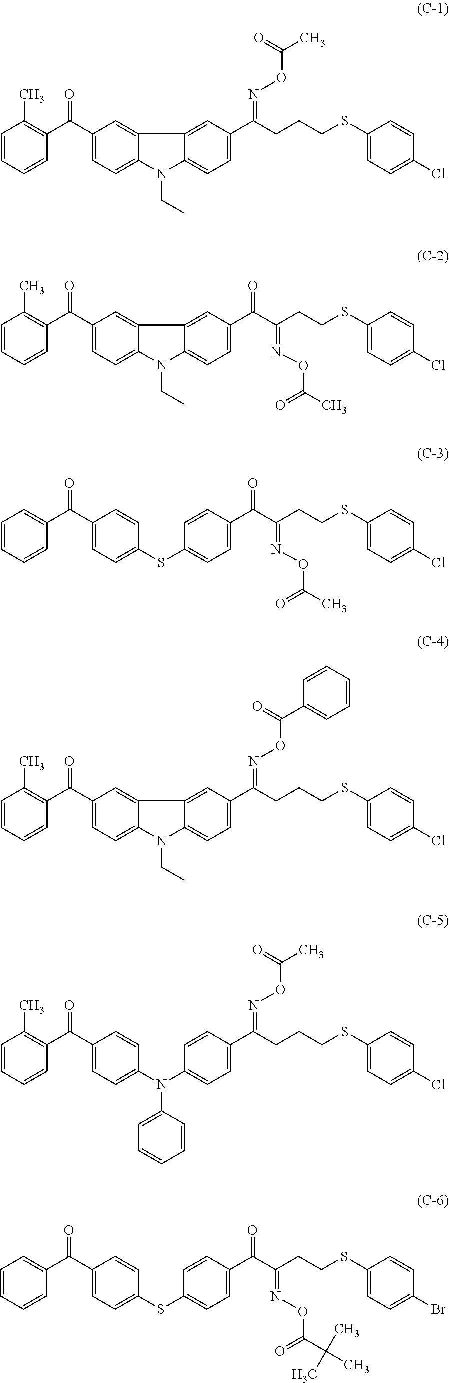 [SMILES: CCn1c2ccc(C(=O)c3ccccc3C)cc2c2cc(/C(CCCSc3ccc(Cl)cc3)=N/OC(C)=O)ccc21 ; CCn1c2ccc(C(=O)/C(CCSc3ccc(Cl)cc3)=N/OC(C)=O)cc2c2cc(C(=O)c3ccccc3C)ccc21 ; CC(=O)O/N=C(\CCSc1ccc(Cl)cc1)C(=O)c1ccc(Sc2ccc(C(=O)c3ccccc3)cc2)cc1 ; CCn1c2ccc(C(=O)c3ccccc3C)cc2c2cc(/C(CCCSc3ccc(Cl)cc3)=N/OC(=O)c3ccccc3)ccc21 ; CC(=O)O/N=C(\CCCSc1ccc(Cl)cc1)c1ccc(N(c2ccccc2)c2ccc(C(=O)c3ccccc3C)cc2)cc1 ; CC(C)(C)C(=O)O/N=C(\CCSc1ccc(Br)cc1)C(=O)c1ccc(Sc2ccc(C(=O)c3ccccc3)cc2)cc1]

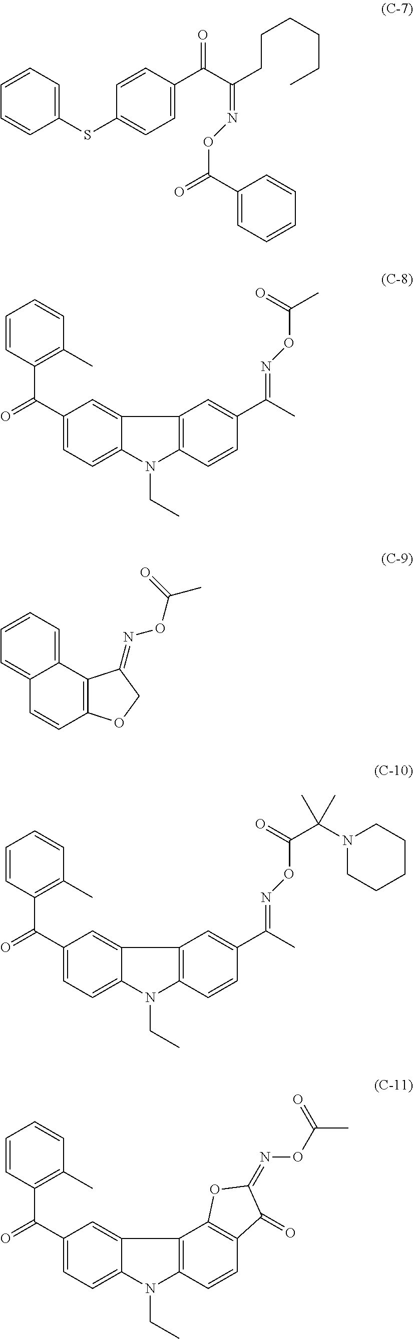 [SMILES: CCCCCC/C(=N/OC(=O)c1ccccc1)C(=O)c1ccc(Sc2ccccc2)cc1 ; CCn1c2ccc(C(=O)c3ccccc3C)cc2c2cc(/C(C)=N/OC(C)=O)ccc21 ; CC(=O)O/N=C1\COc2ccc3ccccc3c21 ; CCn1c2ccc(C(=O)c3ccccc3C)cc2c2cc(/C(C)=N/OC(=O)C(C)(C)N3CCCCC3)ccc21 ; CCn1c2ccc(C(=O)c3ccccc3C)cc2c2c3c(ccc21)C(=O)/C(=N\OC(C)=O)O3]

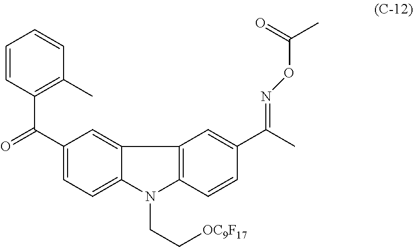

In the present invention, an oxime compound having a fluorine atom can also be used as the photopolymerization initiator. Specific examples of the oxime compound having a fluorine atom include a compound described in JP2010-262028A, Compound 24 and 36 to 40 described in JP2014-500852A, and Compound (C-3) described in JP2013-164471A. The content of which is incorporated herein by reference.

In the present invention, as the photopolymerization initiator, an oxime initiator having a nitro group can be used. It is preferable that the oxime compound having a nitro group is a dimer. Specific examples of the oxime compound having a nitro group include compounds described in paragraphs “0031” to “0047” of JP2013-114249A and paragraphs “0008” to “0012” and “0070” to “0079” of JP2014-137466A, compounds described in paragraphs “0007” to 0025″ of JP4223071B, and ADEKA ARKLS NCI-831 (manufactured by Adeka Corporation).

In the present invention, as the photopolymerization initiator, an oxime compound having a benzofuran skeleton can also be used. Specific examples include compounds OE-01 to OE-75 described in WO2015/036910A.

It is preferable that the photopolymerization initiator includes an oxime compound and an α-aminoketone compound. By using the oxime compound and the α-aminoketone compound in combination, the developability is improved, and a pattern having excellent rectangularity is likely to be formed. In a case where the oxime compound and the α-aminoketone compound are used in combination, the content of the α-aminoketone compound is preferably 50 to 600 parts by mass and more preferably 150 to 400 parts by mass with respect to 100 parts by mass of the oxime compound.

The content of the photopolymerization initiator is preferably 0.1 to 50 mass %, more preferably 0.5 to 30 mass %, and still more preferably 1 to 20 mass % with respect to the total solid content of the composition according to the present invention. In the above-described range, excellent sensitivity and pattern formability can be obtained. The composition according to the present invention may include one photopolymerization initiator or two or more photopolymerization initiators. In a case where the composition includes two or more photopolymerization initiators, it is preferable that the total content of the two or more photopolymerization initiators is in the above-described range.

<<Acid Generator>>

The composition according to the present invention may include an acid generator. In particular, in a case where the composition according to the present invention includes a compound having a cyclic ether group or a cationically polymerizable compound as the crosslinking compound, it is preferable that the composition according to the present invention includes an acid generator. As the acid generator, a compound (photoacid generator) which generates an acid by light irradiation is preferable. Examples of the acid generator include compounds which are decomposed by light irradiation to generate an acid including: an onium salt compound such as a diazonium salt, a phosphonium salt, a sulfonium salt, or an iodonium salt; and a sulfonate compound such as imidosulfonate, oximesulfonate, diazodisulfone, disulfone, or ortho-nitrobenzyl sulfonate. The kind, specific compounds, and preferable examples of the acid generator can be found in the description of a compound in paragraphs “0066” to “0122” of JP2008-13646A, the content of which is also applicable to the present invention.

Examples of a compound which can be preferably used as the acid generator in the present invention include compounds represented by the following Formulae (b1), (b2), and (b3).

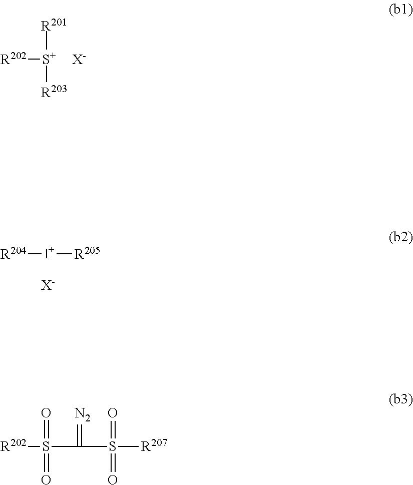

In Formula (b1), R²⁰¹, R²⁰², and R²⁰³ each independently represent an organic group. X⁻ represents a non-nucleophilic anion, preferably a sulfonate anion, a carboxylate anion, a bis(alkylsulfonyl)amide anion, a tris(alkylsulfonyl)methide anion, BF₄⁻, PF₆⁻, or SbF₆⁻, and more preferably BF₄⁻, PF₆⁻, or SbF₆⁻.

Examples of a commercially available product of the acid generator include WPAG-469 (manufactured by Wako Pure Chemical Industries, Ltd.) and CPI-100P (manufactured by San-Apro Ltd.).

The content of the acid generator is preferably 0.1 to 50 mass %, more preferably 0.5 to 30 mass %, and still more preferably 1 to 20 mass % with respect to the total solid content of the composition. The composition according to the present invention may include one acid generator or two or more acid generators. In a case where the composition includes two or more acid generators, it is preferable that the total content of the two or more acid generators is in the above-described range.

<<Crosslinking Aid>>

It is preferable that the composition according to the present invention includes a crosslinking aid in order to promote a reaction of the crosslinking compound. Examples of the crosslinking aid include at least one selected from the group consisting of a polyfunctional thiol, an alcohol, an amine, and a carboxylic acid. The content of the crosslinking aid is preferably 1 to 1000 parts by mass, more preferably 1 to 500 parts by mass, and still more preferably 1 to 200 parts by mass with respect to 100 parts by mass of the crosslinking compound. The composition according to the present invention may include one crosslinking aid or two or more crosslinking aids. In a case where the composition includes two or more crosslinking aids, it is preferable that the total content of the two or more crosslinking aids is in the above-described range.

(Polyfunctional Thiol)

In the present invention, examples of the polyfunctional thiol include a compound having two or more thiol groups in a molecule. The polyfunctional thiol is preferably a secondary alkanethiol and more preferably a compound having a structure represented by the following Formula (T1).

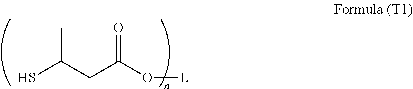

(In Formula (T1), n represents an integer of 2 to 4, and L represents a divalent to tetravalent linking group.)

In Formula (T1), it is preferable that a linking group L is an aliphatic group having 2 to 12 carbon atoms, and it is more preferable that n represents 2 and L represents an alkylene group having 2 to 12 carbon atoms. Specific examples of the polyfunctional thiol include compounds represented by the following Structural Formulae (T2) to (T4). In particular, a compound represented by Structural Formula (T2) is preferable. Among these polyfunctional thiols, one kind may be used alone, or two or more kinds may be used in combination.

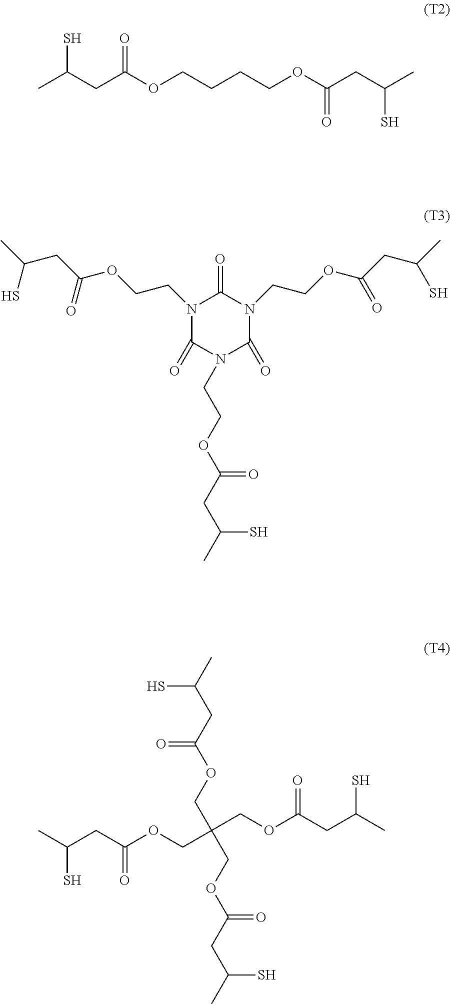

(Amine)

In the present invention, the amine as the crosslinking aid is preferably polyamine and more preferably diamine. Examples of the amine include hexamethylenediamine, triethylenetetramine, and polyethyleneimine.

(Alcohol)

In the present invention, the alcohol as the crosslinking aid is preferably polyhydric alcohol and more preferably diol. Examples of the alcohol include a polyether diol compound, a polyester diol compound, and a polycarbonate diol compound. Specific examples of the alcohol can be found in paragraphs “0128” to “0163” and “0172” of JP2013-253224A, the content of which is incorporated herein by reference.

(Carboxylic Acid)

In the present invention, examples of the carboxylic acid as the crosslinking aid include 3,3′,4,4′-biphenyltetracarboxylic anhydride, maleic acid, phthalic acid, and trimellitic acid.

<<Crosslinking Catalyst>>

The composition according to the present invention may include a crosslinking catalyst. In particular, in a case where the composition according to the present invention includes an alkoxysilyl group or a compound having a chlorosilyl group as the crosslinking compound, a sol-gel reaction is promoted and a strong cured film is obtained by including the crosslinking catalyst. Examples of the crosslinking catalyst include an acid catalyst and a base catalyst. Examples of the acid catalyst include hydrochloric acid, nitric acid, sulfuric acid, sulfurous acid, hydrogen sulfide, perchloric acid, hydrogen peroxide, carbonic acid, a carboxylic acid such as formic acid or acetic acid, a substituted carboxylic acid in which R in a structural formula represented by RCOOH is substituted with another element or a substituent, a sulfonic acid such as benzenesulfonic acid, and phosphoric acid. Further, Lewis acid such as aluminum chloride, aluminum acetylacetonate, zinc chloride, tin chloride, a boron trifluoride diethyl ether complex, or iodotrimethylsilane may be used. In addition, examples of the base catalyst include an ammonia base compound such as ammonia water and an organic amine such as ethylamine or aniline. In addition, as the crosslinking catalyst, a catalyst described in paragraphs “0070” to “0076” of JP2013-201007A can also be used.

The content of the crosslinking catalyst is preferably 0.1 to 100 parts by mass, more preferably 0.1 to 50 parts by mass, and still more preferably 0.1 to 20 parts by mass with respect to 100 parts by mass of the crosslinking compound. The composition according to the present invention may include one crosslinking catalyst or two or more crosslinking catalysts. In a case where the composition includes two or more crosslinking catalysts, it is preferable that the total content of the two or more crosslinking catalysts is in the above-described range.

<<Resin>>

The composition according to the present invention may include a resin. The resin is mixed, for example, in order to disperse the pigment and the like in the composition and to be added as a binder. The resin which is mainly used to disperse the pigments and the like will also be called a dispersant. However, the above-described uses of the resin are merely exemplary, and the resin can be used for purposes other than the uses.

The weight-average molecular weight (Mw) of the resin is preferably 2000 to 2000000. The upper limit is preferably 1000000 or lower and more preferably 500000 or lower. The lower limit is preferably 3000 or higher and more preferably 5000 or higher.

The content of the resin is preferably 10 to 80 mass % and more preferably 20 to 60 mass % with respect to the total solid content of the composition. The composition may include one resin or two or more resins. In a case where the composition includes two or more resins, it is preferable that the total content of the two or more resins is in the above-described range.

(Dispersant)

Examples of the dispersant include: a polymer dispersant such as a resin having an amine group (polyamideamine or a salt thereof), an oligo imine resin, a polycarboxylic acid or a salt thereof, a high-molecular-weight unsaturated acid ester, a modified polyurethane, a modified polyester, a modified poly(meth)acrylate, a (meth)acrylic copolymer, or a naphthalene sulfonic acid formalin condensate; Examples of the oligoimine resin include a compound described in paragraphs “0102” to “0174” of JP2012-255128A.

In terms of a structure, the polymer dispersant can be further classified into a linear polymer, a terminal-modified polymer, a graft polymer, and a block polymer. In addition, as the polymer dispersant, a resin having an acid value of 60 mgKOH/g or higher (more preferably 60 mgKOH/g or higher and 300 mgKOH/g or lower) can be preferably used.

Examples of the terminal-modified polymer include a polymer having a phosphate group at a terminal thereof described in JP1991-112992A (JP-H3-112992A) or JP2003-533455A, a polymer having a sulfo group at a terminal thereof described in JP2002-273191A, and a polymer having a partial skeleton or a heterocycle of an organic colorant described in JP1997-77994A (JP-H9-77994A). In addition, polymers described in JP2007-277514A in which two or more anchor sites (for example, an acid group, a basic group, a partial skeleton or a heterocycle of an organic colorant) to a pigment surface are introduced into a terminal thereof are also preferable due to its dispersion stability.

Examples of the graft polymer include a reaction product of poly(low-alkylene imine) and polyester described in JP1979-37082A (JP-S54-37082A), JP1996-507960A (JP-H8-507960A), or JP2009-258668A, a reaction product of polyallylamine and polyester described in JP1997-169821A (JP-H9-169821A), a copolymer of a macromonomer and a monomer having a nitrogen atom described in JP1998-339949A (JP-H10-339949A) or JP2004-37986A, a graft polymer having a partial skeleton or a heterocycle of an organic colorant described in JP2003-238837A, JP2008-9426A, or JP2008-81732A, and a copolymer of a macromonomer and an acid group-containing monomer described in JP2010-106268A. In addition, a graft copolymer described in paragraphs “0025” to “0094” of JP2012-255128A is also preferable.

As the macromonomer used for manufacturing the graft polymer by radical polymerization, a well-known macromonomer can be used, and examples thereof include macromonomers manufactured by Toagosei Co., Ltd. such as AA-6 (polymethyl methacrylate having a methacryloyl group as a terminal group), AS-6 (polystyrene having a methacryloyl group as a terminal group), AN-6S (a copolymer of styrene and acrylonitrile having a methacryloyl group as a terminal group), and AB-6 (polybutyl acrylate having a methacryloyl group as a terminal group); macromonomers manufactured by Daicel Corporation such as PLACCEL FM5 (an adduct of 2-hydroxyethyl methacrylate and 5 molar equivalents of ϵ-caprolactone) and FA10L (an adduct of 2-hydroxyethyl acrylate and 10 molar equivalents of ϵ-caprolactone); and a polyester macromonomer described in JP1990-272009A (JP-H2-272009A). Among these, from the viewpoint of the dispersibility and dispersion stability of the pigment dispersion and the developability of the composition in which the pigment dispersion is used, a polyester macromonomer having excellent flexibility and solvent compatibility is more preferable, and the polyester macromonomer represented by the polyester macromonomer described in JP1990-272009A (JP-H2-272009A) is most preferable.

As the block polymer, a block polymer described in JP2003-49110A or JP2009-52010A is preferable.

The resin (dispersant) is available as a commercially available product, and specific examples thereof include: “Disperbyk-101 (polyamideamine phosphate), 107 (carboxylate), 110, 111 (copolymer containing an acid group), 130 (polyamide), 161, 162, 163, 164, 165, 166, and 170 (high molecular weight copolymer)” and “BYK-P104, P105 (high molecular weight unsaturated polycarboxylic acid)” all of which are manufactured by BYK Chemie; “EFKA 4047, 4050 to 4165 (polyurethane compound), EFKA 4330 to 4340 (block copolymer), 4400 to 4402 (modified polyacrylate), 5010 (polyester amide), 5765 (high molecular weight polycarboxylate), 6220 (fatty acid polyester), 6745 (phthalocyanine derivative), and 6750 (azo pigment derivative)” all of which are manufactured by EFKA; “AJISPER PB821, PB822, PB880, and PB881” all of which are manufactured by Ajinomoto Fine Techno Co., Inc.; “FLOWLEN TG-710 (urethane oligomer)” and “POLYFLOW No. 50E and No. 300 (acrylate copolymer)” all of which are manufactured by Kyoeisha Chemical Co., Ltd.; “DISPARLON KS-860, 873SN, 874, #2150 (aliphatic polycarboxylic acid), #7004 (polyether ester), DA-703-50, DA-705, and DA-725” all of which are manufactured by Kusumoto Chemicals Ltd.; “DEMOL RN, N (naphthalene sulfonic acid formalin polycondensate), MS, C, and SN-B (aromatic sulfonic acid formalin polycondensate)”, “HOMOGENOL L-18 (high molecular polycarboxylic acid)”, “EMULGEN 920, 930, 935, and 985 (polyoxyethylene nonylphenyl ether)”, and “ACETAMIN 86 (stearylamine acetate)” all of which are manufactured by Kao Corporation; “SOLSPERSE 5000 (phthalocyanine derivative), 22000 (azo pigment derivative), 13240 (polyester amine), 3000, 17000, 27000 (polymer having a functional group at a terminal thereof), 24000, 28000, 32000, and 38500 (graft polymer)” all of which are manufactured by Lubrizol Corporation; “NIKKOL T106 (polyoxyethylene sorbitan monooleate) and MYS-IEX (polyoxyethylene monostearate)” all of which manufactured by Nikko Chemicals Co., Ltd.; HINOACT T-8000E manufactured by Kawaken Fine Chemicals Co., Ltd.; “EFKA-46, EFKA-47, EFKA-47EA, EFKA POLYMER 100, EFKA POLYMER 400, EFKA POLYMER 401, and EFKA POLYMER 450” all of which are manufactured by Morishita Co., Ltd. and “DISPERSE AID 6, DISPERSE AID 8, DISPERSE AID 15, and DISPERSE AID 9100” all of which are manufactured by San Nopco Limited; “ADEKA PLURONIC L31, F38, L42, L44, L61, L64, F68, L72, P95, F77, P84, F87, P94, L101, P103, F108, L121, and P-123” all of which are manufactured by Adeka Corporation; and “IONET S-20” manufactured by Sanyo Chemical Industries Ltd.

Among these resins, one kind may be used alone, or two or more kinds may be used in combination. In addition, an alkali-soluble resin described below can also be used as the dispersant. Examples of the alkali-soluble resin include a (meth)acrylic acid copolymer, an itaconic acid copolymer, a crotonic acid copolymer, a maleic acid copolymer, a partially esterified maleic acid copolymer, an acidic cellulose derivative having a carboxyl group at a side chain thereof, and a resin obtained by modifying a polymer having a hydroxyl group with an acid anhydride. Among these, a (meth)acrylic acid copolymer is preferable. In addition, an N-position-substituted maleimide monomer copolymer described in JP1998-300922A (JP-H10-300922A), an ether dimer copolymer described in JP2004-300204A, or an alkali-soluble resin having a polymerizable group described in JP1995-319161A (JP-H7-319161A) is also preferable.

The content of the dispersant is preferably 1 to 80 parts by mass, more preferably 5 to 70 parts by mass, and still more preferably 10 to 60 parts by mass with respect to 100 parts by mass of the pigment.

(Alkali-Soluble Resin)

The composition according to the present invention can include an alkali-soluble resin as a resin. By the composition including the alkali-soluble resin, developability and pattern formability is improved. The alkali-soluble resin can also be used as the dispersant or the binder. In a case where a pattern is not formed, the alkali-soluble resin may not be used.

The molecular weight of the alkali-soluble resin is not particularly limited, and the weight-average molecular weight (Mw) thereof is preferably 5000 to 100000. In addition, the number-average molecular weight (Mn) of the alkali-soluble resin is preferably 1000 to 20000.

The alkali-soluble resin may be a linear organic polymer and can be appropriately selected from alkali-soluble resins having at least one group for promoting alkali dissolution in a molecule (preferably a molecule having an acrylic copolymer or a styrene copolymer as a main chain).

As the alkali-soluble resin, from the viewpoint of heat resistance, a polyhydroxystyrene resin, a polysiloxane resin, an acrylic resin, an acrylamide resin, or an acryl/acrylamide copolymer resin is preferable, and from the viewpoint of controlling developability, an acrylic resin, an acrylamide resin, or an acryl/acrylamide copolymer resin is preferable.

Examples of the group for promoting alkali dissolution (hereinafter, also referred to as the acid group) include a carboxyl group, a phosphate group, a sulfo group, and a phenolic hydroxyl group. Among these, a carboxyl group is preferable. Among these acid groups, one kind may be used alone, or two or more kinds may be used in combination.

As the alkali-soluble resin, a polymer having a carboxyl group at a side chain thereof is preferable, and examples thereof include: an alkali-soluble phenol resin such as a methacrylic acid copolymer, an acrylic acid copolymer, an itaconic acid copolymer, a crotonic acid copolymer, a maleic acid copolymer, a partially esterified maleic acid copolymer, or a novolac type resin; an acidic cellulose derivative having a carboxyl group at a side chain thereof; and a resin obtained by adding an acid anhydride to a polymer having a hydroxyl group. In particular, a copolymer of (meth)acrylic acid and another monomer which is copolymerizable with the (meth)acrylic acid is preferable as the alkali-soluble resin. Examples of the monomer which is copolymerizable with the (meth)acrylic acid include an alkyl (meth)acrylate, an aryl (meth)acrylate, and a vinyl compound. Examples of the alkyl (meth)acrylate and the aryl (meth)acrylate include methyl (meth)acrylate, ethyl (meth)acrylate, propyl (meth)acrylate, butyl (meth)acrylate, isobutyl (meth)acrylate, pentyl (meth)acrylate, hexyl (meth)acrylate, octyl (meth)acrylate, phenyl (meth)acrylate, benzyl (meth)acrylate, tolyl (meth)acrylate, naphthyl (meth)acrylate, and cyclohexyl (meth)acrylate. Examples of the vinyl compound include styrene, α-methylstyrene, vinyl toluene, glycidyl methacrylate, acrylonitrile, vinyl acetate, N-vinylpyrrolidone, tetrahydrofurfuryl methacrylate, a polystyrene macromonomer, and a polymethyl methacrylate macromonomer. As other monomers, a N-position-substituted maleimide monomer described in JP1998-300922A (H10-300922A) (for example, N-phenylmaleimide or N-cyclohexylmaleimide) can also be used. Among these monomers which are copolymerizable with the (meth)acrylic acid, one kind may be used alone, or two or more kinds may be used in combination.

As the alkali-soluble resin, a copolymer including benzyl (meth)acrylate and (meth)acrylic acid; a copolymer including benzyl (meth)acrylate, (meth)acrylic acid, and 2-hydroxyethyl (meth)acrylate; or a multi-component copolymer including benzyl (meth)acrylate, (meth)acrylic acid, and another monomer can be preferably used. In addition, copolymers described in JP1995-140654A (JP-H7-140654A) obtained by copolymerization of 2-hydroxyethyl (meth)acrylate can be preferably used, and examples thereof include: a copolymer including 2-hydroxypropyl (meth)acrylate, a polystyrene macromonomer, benzyl methacrylate, and methacrylic acid; a copolymer including 2-hydroxy-3-phenoxypropyl acrylate, a polymethyl methacrylate macromonomer, benzyl methacrylate, and methacrylic acid; a copolymer including 2-hydroxyethyl methacrylate, a polystyrene macromonomer, methyl methacrylate, and methacrylic acid; or a copolymer including 2-hydroxyethyl methacrylate, a polystyrene macromonomer, benzyl methacrylate, and methacrylic acid. In addition, as a commercially available product, for example, FF-426 (manufactured by Fujikura Kasei Co., Ltd.) can also be used.

As the alkali-soluble resin, a polymer obtained by copolymerization of monomer components including a compound represented by the following Formula (ED1) and/or a compound represented by the following Formula (ED2) (hereinafter, these compounds will also be referred to as “ether dimer”) is also preferable.

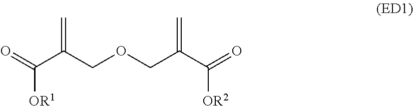

In Formula (ED1), R¹ and R² each independently represent a hydrogen atom or a hydrocarbon group having 1 to 25 carbon atoms which may have a substituent.

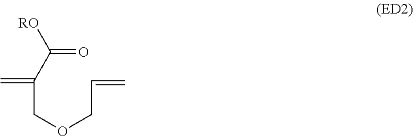

In Formula (ED2), R represents a hydrogen atom or an organic group having 1 to 30 carbon atoms. Specific examples of Formula (ED2) can be found in the description of JP2010-168539A

Specific examples of the ether dimer can be found in paragraph “0317” of JP2013-29760A, the content of which is incorporated herein by reference. Among these ether dimers, one kind may be used alone, or two or more kinds may be used in combination.

The alkali-soluble resin may include a structural unit which is derived from a compound represented by the following Formula (X).

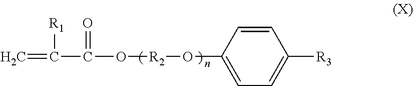

In Formula (X), R₁ represents a hydrogen atom or a methyl group, R₂ represents an alkylene group having 2 to 10 carbon atoms, and R₃ represents a hydrogen atom or an alkyl group having 1 to 20 carbon atoms which may have a benzene ring. n represents an integer of 1 to 15.

In Formula (X), the number of carbon atoms in the alkylene group of R₂ is preferably 2 to 3. In addition, the number of carbon atoms in the alkyl group of R₃ is preferably 1 to 20 and more preferably 1 to 10, and the alkyl group of R₃ may have a benzene ring. Examples of the alkyl group having a benzene ring represented by R₃ include a benzyl group and a 2-phenyl(iso)propyl group.

The details of the alkali-soluble resin can be found in paragraphs “0558” to “0571” of JP2012-208494A (corresponding to paragraphs “0685” to “0700” of US2012/0235099A), the content of which is incorporated herein by reference.

Further, a copolymer (B) described in paragraphs “0029” to “0063” and an alkali-soluble resin used in Examples of JP2012-32767A, a binder resin described in paragraphs “0088” to “0098” and a binder resin used in Examples of JP2012-208474A, a binder resin described in paragraphs “0022” to “0032 and a binder resin used in Examples of JP2012-137531A, a binder resin described in paragraphs “0132” to “0143” and a binder resin used in Examples of JP2013-024934A, a binder resin described in paragraphs “0092” to “0098” and Examples of JP2011-242752A, or a binder resin described in paragraphs “0030” to “0072” of JP2012-032770A can also be used. The content of which is incorporated herein by reference.

The acid value of the alkali-soluble resin is preferably 30 to 500 mgKOH/g. The lower limit is more preferably 50 mgKOH/g or higher and still more preferably 70 mgKOH/g or higher. The upper limit is more preferably 400 mgKOH/g or lower, still more preferably 200 mgKOH/g or lower, even still more preferably 150 mgKOH/g or lower, and even yet still more preferably 120 mgKOH/g or lower.

The content of the alkali-soluble resin is preferably 0.1 to 50 mass % with respect to the total solid content of the composition. The lower limit is preferably 0.5 mass % or higher, more preferably 1 mass % or higher, still more preferably 2 mass % or higher, and even still more preferably 3 mass % or higher. The upper limit is more preferably 30 mass % or lower, and still more preferably 10 mass % or lower. The composition according to the present invention may include one alkali-soluble resin or two or more alkali-soluble resins. In a case where the composition includes two or more alkali-soluble resins, it is preferable that the total content of the two or more alkali-soluble resins is in the above-described range.

<<Other Resins>>

It is also preferable that the resin is a resin having a repeating unit represented by any one of Formulae (A3-1) to (A3-7).

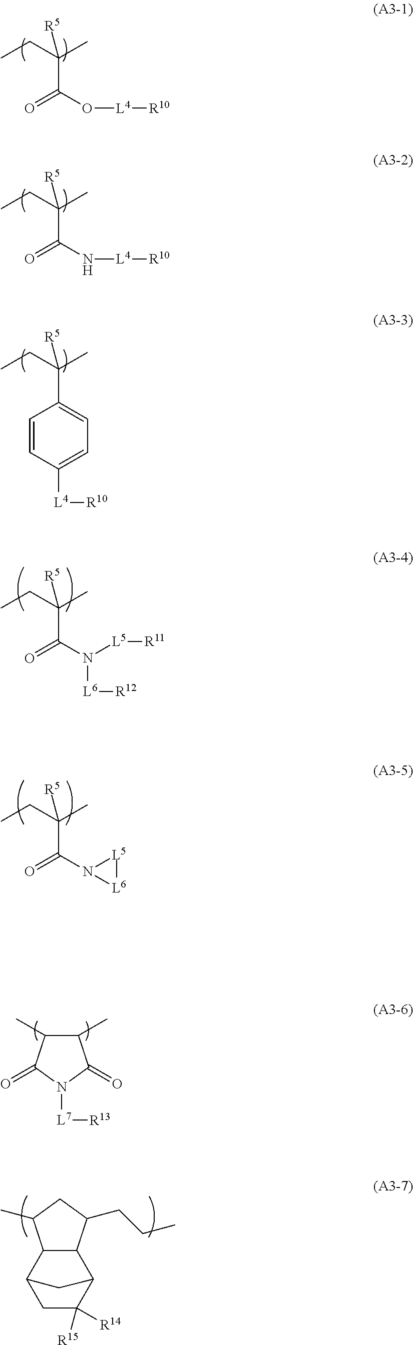

In the formula, R⁵ represents a hydrogen atom or an alkyl group, L⁴ to L⁷ each independently represent a single bond or a divalent linking group, R¹⁰ to R¹³ each independently represent an alkyl group or an aryl group, and R¹⁴ and R¹⁵ each independently represent a hydrogen atom or a substituent.

R⁵ represents a hydrogen atom or an alkyl group. The number of carbon atoms in the alkyl group is preferably 1 to 5, more preferably 1 to 3, and still more preferably 1. It is preferable that R⁵ represents a hydrogen atom or a methyl group.

L⁴ to L⁷ each independently represent a single bond or a divalent linking group. Examples of the divalent linking group include an alkylene group, an arylene group, —O—, —S—, —CO—, —COO—, —OCO—, —SO₂—, —NR¹⁰— (R¹⁰ represents a hydrogen atom or an alkyl group and preferably a hydrogen atom), and a group including a combination thereof. Among these, a group including a combination —O— and at least one of an alkylene group, an arylene group, or an alkylene group is preferable. The number of carbon atoms in the alkylene group is preferably 1 to 30, more preferably 1 to 15, and still more preferably 1 to 10. The alkylene group may have a substituent but is preferably unsubstituted. The alkylene group may be linear, branched, or cyclic. In addition, the cyclic alkylene group may be monocyclic or polycyclic. The number of carbon atoms in the arylene group is preferably 6 to 18, more preferably 6 to 14, and still more preferably 6 to 10.

The alkyl group represented by R¹⁰ may be linear, branched, or cyclic and is preferably cyclic. The alkyl group may have a substituent or may be unsubstituted. The number of carbon atoms in the alkyl group is preferably 1 to 30, more preferably 1 to 20, and still more preferably 1 to 10. The number of carbon atoms in the aryl group represented by R¹⁰ is preferably 6 to 18, more preferably 6 to 12, and still more preferably 6. It is preferable that R¹⁰ represents a cyclic alkyl group or an aryl group.

The alkyl group represented by R¹¹ and R¹² may be linear, branched, or cyclic and is preferably linear or branched. The alkyl group may have a substituent or may be unsubstituted. The number of carbon atoms in the alkyl group is preferably 1 to 12, more preferably 1 to 6, and still more preferably 1 to 4. The number of carbon atoms in the aryl group represented by R¹¹ and R¹² is preferably 6 to 18, more preferably 6 to 12, and still more preferably 6. It is preferable that R¹¹ and R¹² represent a linear or branched alkyl group.

The alkyl group represented by R¹³ may be linear, branched, or cyclic and is preferably linear or branched. The alkyl group may have a substituent or may be unsubstituted. The number of carbon atoms in the alkyl group is preferably 1 to 12, more preferably 1 to 6, and still more preferably 1 to 4. The number of carbon atoms in the aryl group represented by R¹³ is preferably 6 to 18, more preferably 6 to 12, and still more preferably 6. It is preferable that R¹³ represents a linear or branched alkyl group or an aryl group.

Examples of the substituent represented by R¹⁴ and R¹⁵ include the groups described above regarding R_{Z} in Formula (2). In particular, it is preferable that at least one of R¹⁴ or R¹⁵ represents a cyano group or —COORa. Ra represents a hydrogen atom or a substituent. Examples of the substituent represented by Ra include the groups described above regarding R_{Z} in Formula (2). As Ra, for example, an alkyl group or an aryl group is preferable.

Examples of a commercially available product of the resin having a repeating unit represented by Formula (A3-7) include ARTON F4520 (manufactured by JSR Corporation).

<<Solvent>>

The composition according to the present invention may include a solvent. Examples of the solvent include water and an organic solvent. Basically, the solvent is not particularly limited as long as it satisfies the solubility of each component and the coating properties of the composition. However, it is preferable that the organic solvent is selected in consideration of the coating properties and safety of the composition.

Preferable examples of the organic solvent are the following organic solvents:
an ester, for example, ethyl acetate, n-butyl acetate, isobutyl acetate, cyclohexyl acetate, amyl formate, isoamyl acetate, isobutyl acetate, butyl propionate, isopropyl butyrate, ethyl butyrate, butyl butyrate, methyl lactate, ethyl lactate, an alkyl oxyacetate (for example, methyl oxyacetate, ethyl oxyacetate, or butyl oxyacetate (for example, methyl methoxyacetate, ethyl methoxyacetate, butyl methoxyacetate, methyl ethoxyacetate, or ethyl ethoxyacetate)), a 3-oxypropionic acid alkyl ester (for example, 3-methyl oxypropionate or 3-ethyl oxypropionate (for example, methyl 3-methoxypropionate, ethyl 3-methoxypropionate, methyl 3-ethoxypropionate, or ethyl 3-ethoxypropionate)), a 2-oxypropionic acid alkyl ester (for example, methyl 2-oxypropionate, ethyl 2-oxypropionate, or propyl 2-oxypropionate (for example, methyl 2-methoxypropionate, ethyl 2-methoxypropionate, propyl 2-methoxypropionate, methyl 2-ethoxypropionate, or ethyl 2-ethoxypropionate)), methyl 2-oxy-2-methylpropionate or ethyl 2-oxy-2-methylpropionate (for example, methyl 2-methoxy-2-methylpropionate or ethyl 2-ethoxy-2-methylpropionate), methyl pyruvate, ethyl pyruvate, propyl pyruvate, methyl acetoacetate, ethyl acetoacetate, methyl 2-oxobutanoate or ethyl 2-oxobutanoate;

an ether, for example, diethylene glycol dimethyl ether, tetrahydrofuran, ethylene glycol monomethyl ether, ethylene glycol monoethyl ether, methyl cellosolve acetate, ethyl cellosolve acetate, diethylene glycol monomethyl ether, diethylene glycol monoethyl ether, diethylene glycol monobutyl ether, propylene glycol monomethyl ether, propylene glycol monomethyl ether acetate, propylene glycol monoethyl ether acetate, or propylene glycol monopropyl ether acetate;

a ketone, for example, methyl ethyl ketone, cyclohexanone, cyclopentanone, 2-heptanone, or 3-heptanone; and an aromatic hydrocarbon, for example, toluene or xylene. In this case, it may be preferable that the content of the aromatic hydrocarbon (for example, benzene, toluene, xylene, or ethylbenzene) as the solvent is low (for example, 50 mass parts per million (ppm) or lower, 10 mass ppm or lower, or 1 mass ppm or lower with respect to the total mass of the organic solvent) in consideration of environmental aspects and the like.

Among these organic solvents, one kind may be used alone, or two or more kinds may be used in combination.

In a case where two or more organic solvents are used in combination, in particular, a mixed solution is preferable, the mixed solution including two or more organic solvents selected from the group consisting of methyl 3-ethoxypropionate, ethyl 3-ethoxypropionate, ethyl cellosolve acetate, ethyl lactate, diethylene glycol dimethyl ether, butyl acetate, methyl 3-methoxypropionate, 2-heptanone, cyclohexanone, ethyl carbitol acetate, butyl carbitol acetate, propylene glycol methyl ether, and propylene glycol methyl ether acetate.

In the present invention, the content of a peroxide in the organic solvent is preferably 0.8 mmol/L or lower and more preferably substantially 0 mmol/L.

In the present invention, as the solvent, a solvent having a low metal content is preferably used. For example, the metal content in the solvent is preferably 10 parts mass per billion (ppb) or lower. Optionally, a solvent having a metal content at a mass parts per trillion (ppt) level may be used. For example, such a high-purity solvent is available from Toyo Gosei Co., Ltd. (The Chemical Daily, Nov. 13, 2015).

Examples of a method of removing impurities such as metal from the solvent include distillation (for example, molecular distillation or thin-film distillation) and filtering using a filter. The pore size of a filter used for the filtering is preferably 10 nm or less, more preferably 5 nm or less, and still more preferably 3 nm or less. As a material of the filter, polytetrafluoroethylene, polyethylene, or nylon is preferable.

The solvent may include an isomer (a compound having the same number of atoms and a different structure). In addition, the organic solvent may include only one isomer or a plurality of isomers.

The content of the solvent is preferably 10 to 90 mass %, more preferably 20 to 80 mass %, and still more preferably 25 to 75 mass % with respect to the total mass of the composition.

<<Polymerization Inhibitor>>

The composition according to the present invention may include a polymerization inhibitor in order to prevent unnecessary thermal polymerization of the crosslinking compound during the manufacturing or storage of the composition. Examples of the polymerization inhibitor include a phenol hydroxyl group-containing compound, a N-oxide compound, a piperidine-1-oxyl free-radical compound, a pyrrolidine-1-oxyl free-radical compound, a N-nitrosophenylhydroxyamine, a diazonium compound, a cationic dye, a sulfide group-containing compound, a nitro group-containing compound, a phosphorus compound, a lactone compound, and a transition metal compound (for example, FeCl₃ or CuCl₂). In addition, the compounds may be composite compounds in which a plurality of structures which exhibit a polymerization inhibition function such as a phenol skeleton or a phosphorus-containing skeleton are present in the same molecule. For example, a compound described in JP1998-46035A (JP-H10-46035A) is also preferably used. Specific examples of the polymerization inhibitor include hydroquinone, p-methoxyphenol, di-t-butyl-p-cresol, pyrogallol, t-butylcatechol, benzoquinone, 4,4′-thiobis(3-methyl-6-t-butylphenol), 2,2′-methylenebis(4-methyl-6-t-butylphenol), and N-nitrosophenylhydroxyamine cerium (III) salt. Among these, p-methoxyphenol is preferable.

The content of the polymerization inhibitor is preferably 0.01 to 5 mass % with respect to the total solid content of the composition.

<<Surfactant>>

The composition according to the present invention may include various surfactants from the viewpoint of further improving coating properties. As the surfactants, various surfactants such as a fluorine surfactant, a nonionic surfactant, a cationic surfactant, an anionic surfactant, or a silicone surfactant can be used.

By the composition including a fluorine surfactant, liquid characteristics (for example, fluidity) of a coating solution prepared from the composition are further improved, and the uniformity in coating thickness and liquid saving properties can be further improved. That is, in a case where a film is formed using a coating solution prepared using the composition including a fluorine surfactant, the interfacial tension between a coated surface and the coating solution decreases, the wettability on the coated surface is improved, and the coating properties on the coated surface are improved. Therefore, a film having a uniform thickness with reduced unevenness in thickness can be formed more suitably.

The fluorine content in the fluorine surfactant is preferably 3 to 40 mass %, more preferably 5 to 30 mass %, and still more preferably 7 to 25 mass %. The fluorine surfactant in which the fluorine content is in the above-described range is effective from the viewpoints of the uniformity in the thickness of the coating film and liquid saving properties, and the solubility thereof in the composition is also excellent.

Examples of the fluorine surfactant include: MEGAFACE F171, F172, F173, F176, F177, F141, F142, F143, F144, R30, F437, F475, F479, F482, F554, F780, and RS-72-K (all of which are manufactured by DIC Corporation); FLUORAD FC430, FC431, and FC171 (all of which are manufactured by Sumitomo 3M Ltd.); SURFLON S-382, SC-101, SC-103, SC-104, SC-105, SC1068, SC-381, SC-383, S-393, and KH-40 (all of which are manufactured by Asahi Glass Co., Ltd.); and POLYFOX PF636, PF656, PF6320, PF6520, and PF7002 (all of which are manufactured by OMNOVA Solutions Inc.).

As the fluorine surfactant, a compound described in paragraphs “0015” to “0158” of JP2015-117327A can also be used. As the fluorine surfactant, a block polymer can also be used, and specific examples thereof include a compound described in JP2011-89090A.

As the fluorine surfactant, a fluorine-containing polymer compound can be preferably used, the fluorine-containing polymer compound including: a repeating unit derived from a (meth)acrylate compound having a fluorine atom; and a repeating unit derived from a (meth)acrylate compound having 2 or more (preferably 5 or more) alkyleneoxy groups (preferably an ethyleneoxy group and a propyleneoxy group). For example, the following compound can also be used as the fluorine surfactant used in the present invention.

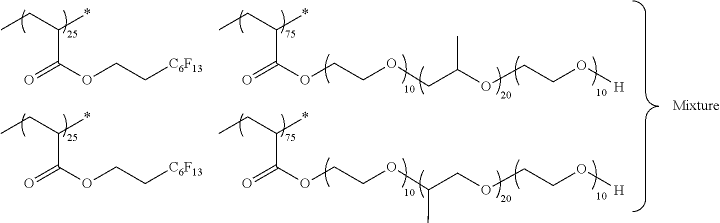

The weight-average molecular weight of the compound is preferably 3000 to 50000 and, for example, 14000.

As the fluorine surfactant, a fluorine-containing polymer having an ethylenically unsaturated group at a side chain can also be preferably used. Specific examples include compounds described in paragraphs “0050” of “0090” and paragraphs “0289” to “0295” of JP2010-164965A, for example, MEGAFACE RS-101, RS-102, and RS-718K manufactured by DIC Corporation.

In addition, as the fluorine surfactant, an acrylic compound in which, when heat is applied to a molecular structure which has a functional group having a fluorine atom, the functional group having a fluorine atom is cut and a fluorine atom is vaporized can also be preferably used. Examples of the fluorine surfactant include MEGAFACE DS series (manufactured by DIC Corporation, The Chemical Daily, Feb. 22, 2016, Nikkei Business Daily, Feb. 23, 2016), for example, MEGAFACE DS-21.

Examples of the nonionic surfactant include glycerol, trimethylolpropane, trimethylolethane, an ethoxylate and a propoxylate thereof (for example, glycerol propoxylate or glycerin ethoxylate), polyoxyethylene lauryl ether, polyoxyethylene stearyl ether, polyoxyethylene oleyl ether, polyoxyethylene octylphenyl ether, polyoxyethylene nonylphenyl ether, polyethylene glycol dilaurate, polyethylene glycol distearate, and sorbitan fatty acid esters (PLURONIC L10, L31, L61, L62, 10R5, 17R2, and 25R2 and TETRONIC 304, 701, 704, 901, 904, and 150R1 (all of which are manufactured by BASF SE)); SOLSPERSE 20000 (manufactured by Lubrication Technology Inc.); NCW-101, NCW-1001, and NCW-1002 (all of which are manufactured by Wako Pure Chemical Industries, Ltd.); PIONIN D-6112, D-6112-W, and D-6315 (all of which are manufactured by Takemoto Oil & Fat Co., Ltd.); and OLFIN E1010, SURFYNOL 104, 400, and 440 (all of which are manufactured by Nissin Chemical Co., Ltd.).

Examples of the cationic surfactant include an organosiloxane polymer KP341 (manufactured by Shin-Etsu Chemical Co., Ltd.), a (meth)acrylic acid (co)polymer POLYFLOW No. 75, No. 90, or No. 95 (manufactured by Kyoeisha Chemical Co., Ltd.), and W001 (manufactured by Yusho Co., Ltd.).

Specific examples of the anionic surfactant include W004, W005, and W017 (manufactured by Yusho Co., Ltd.), and SANDET BL (manufactured by Sanyo Chemical Industries Ltd.).

Examples of the silicone surfactant include: TORAY SILICONE DC3PA, TORAY SILICONE SH7PA, TORAY SILICONE DC11PA, TORAY SILICONE SH21PA, TORAY SILICONE SH28PA, TORAY SILICONE SH29PA, TORAY SILICONE SH30PA, and TORAY SILICONE SH8400 (all of which are manufactured by Dow Corning Corporation); TSF-4440, TSF-4300, TSF-4445, TSF-4460, and TSF-4452 (all of which are manufactured by Momentive Performance Materials Inc.); KP341, KF6001, and KF6002 (all of which are manufactured by Shin-Etsu Chemical Co., Ltd.); and BYK307, BYK323, and BYK330 (all of which are manufactured by BYK-Chemie Japan K.K.).

Among these surfactants, one kind may be used alone, or two or more kinds may be used in combination.

The content of the surfactant is preferably 0.001 to 2.0 mass % and more preferably 0.005 to 1.0 mass % with respect to the total solid content of the composition.

<<Ultraviolet Absorber>>

The composition according to the present invention may include an ultraviolet absorber. The ultraviolet absorber is preferably a conjugated diene compound and more preferably a compound represented by the following Formula (1). In a case where this conjugated diene compound is used, a variation in development performance especially after low-illuminance exposure can be suppressed, and the dependence on exposure illuminance relating to pattern formability such as a line width of a pattern, a film thickness, or an optical spectrum can be more effectively suppressed.

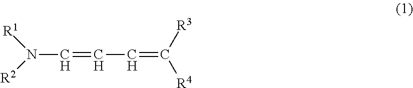

R¹ and R² each independently represent a hydrogen atom, an alkyl group having 1 to 20 carbon atoms, or an aryl group having 6 to 20 carbon atoms, and may be the same as or different from each other but does not represent a hydrogen atom at the same time.

R³ and R⁴ represent an electron-withdrawing group. The Hammett substituent constant σₚ value (hereinafter, referred to simply as “σₚ value”) of the electron-withdrawing group is preferably 0.30 to 1.0 and more preferably 0.30 to 0.8. R³ and R⁴ represent preferably an acyl group, a carbamoyl group, an alkyloxycarbonyl group, an aryloxycarbonyl group, a cyano group, a nitro group, an alkylsulfonyl group, an arylsulfonyl group, a sulfonyloxy group, or a sulfamoyl group, and more preferably an acyl group, a carbamoyl group, an alkyloxycarbonyl group, an aryloxycarbonyl group, a cyano group, an alkylsulfonyl group, an arylsulfonyl group, a sulfonyloxy group, or a sulfamoyl group.

Formula (1) can be found in paragraphs “0148” to “0158” of JP2010-049029A, the content of which is incorporated herein by reference.

Specific examples of the compound represented by Formula (1) include the following compounds. Other examples of the compound represented by Formula (1) include a compound described in paragraphs “0160” to “0162” of JP2010-049029A, the content of which is incorporated herein by reference.

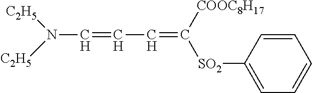

Examples of a commercially available product of the ultraviolet absorber include UV503 (manufactured by Daito Chemical Co., Ltd.).

As the ultraviolet absorber, an ultraviolet absorber such as an amino diene compound, a salicylate compound, a benzophenone compound, a benzotriazole compound, an acrylonitrile compound, or a triazine compound can be preferably used. Specifically, a compound described in JP2013-68814A can be used. As the benzotriazole compound, MYUA series (manufactured by Miyoshi Oil & Fat Co., Ltd.; (The Chemical Daily, Feb. 1, 2016) may be used.

The content of the ultraviolet absorber is preferably 0.01 to 10 mass % and more preferably 0.01 to 5 mass % with respect to the total solid content of the composition according to the present invention.

<<Other Components>>

The infrared absorbing composition according to the present invention may include various additives such as a chain transfer agent, a thermal polymerization initiator, a thermal polymerization component, a plasticizer, a developability improving agent, an antioxidant, an aggregation inhibitor, or a filler.

<Method of Preparing Composition According to Present Invention>

The composition according to the present invention can be prepared by mixing the above-described components with each other. During the preparation of the composition, the respective components may be mixed with each other collectively, or may be mixed with each other sequentially after dissolved and/or dispersed in a solvent. In addition, during mixing, the order of addition or working conditions are not particularly limited. For example, all the components may be dissolved and/or dispersed in a solvent at the same time to prepare the composition. Optionally, two or more solutions or dispersions may be appropriately prepared using the respective components, and the solutions or dispersions may be mixed with each other during use (during application) to prepare the composition.

In addition, in a case where a composition including particles is prepared, it is preferable that a process of dispersing the particles is provided. Examples of a mechanical force used for dispersing the particles in the process of dispersing the particles include compression, squeezing, impact, shearing, and cavitation. Specific examples of the process include a beads mill, a sand mill, a roll mill, a ball mill, a paint shaker, a Microfluidizer, a high-speed impeller, a sand grinder, a project mixer, high-pressure wet atomization, and ultrasonic dispersion. During the pulverization of the particles using a sand mill (beads mill), it is preferable that the process is performed under conditions for increasing the pulverization efficiency, for example, by using beads having a small size and increasing the filling rate of the beads. In addition, it is preferable that rough particles are removed by filtering, centrifugal separation, and the like. In addition, as the process and the disperser for dispersing the particles, a process and a disperser described in “Complete Works of Dispersion Technology, Johokiko Co., Ltd., Jul. 15, 2005”, “Dispersion Technique focusing on Suspension (Solid/Liquid Dispersion) and Practical Industrial Application, Comprehensive Reference List, Publishing Department of Management Development Center, Oct. 10, 1978”, and paragraph “0022” JP2015-157893A can be suitably used. In addition, in the process of dispersing the particles, particles may be refined in a salt milling step. A material, a device, a process conditions, and the like used in the salt milling step can be found in, for example, JP2015-194521A and JP2012-046629A, the content of which is incorporated herein by reference.

During the preparation of the composition, it is preferable that the composition is filtered through a filter, for example, in order to remove foreign matter or to reduce defects. As the filter, any filter which is used in the related art for filtering or the like can be used without any particular limitation. Examples of a material of the filter include: a fluororesin such as polytetrafluoroethylene (PTFE); a polyamide resin such as nylon (for example, nylon-6 or nylon-6,6); and a polyolefin resin (including a polyolefin resin having a high density and an ultrahigh molecular weight) such as polyethylene or polypropylene (PP). Among these materials, polypropylene (including high-density polypropylene) or nylon is preferable.

The pore size of the filter is suitably about 0.01 to 7.0 μm and is preferably about 0.01 to 3.0 μm and more preferably about 0.05 to 0.5 μm. In the above-described range, fine foreign matter, which inhibits a fine and smooth composition in the next step, can be reliably removed. In addition, a fibrous filter material is also preferably used, and examples of the filter material include polypropylene fiber, nylon fiber, and glass fiber. Specifically, a filter cartridge of SBP type series (manufactured by Roki Techno Co., Ltd.; for example, SBP008), TPR type series (for example, TPR002 or TPR005), SHPX type series (for example, SHPX003), or the like can be used.

In a filter is used, a combination of different filters may be used. At this time, the filtering using a first filter may be performed once, or twice or more.

In addition, a combination of first filters having different pore sizes in the above-described range may be used. Here, the pore size of the filter can refer to a nominal value of a manufacturer of the filter. A commercially available filter can be selected from various filters manufactured by Pall Corporation (for example, DFA4201NXEY), Toyo Roshi Kaisha, Ltd., Entegris Japan Co., Ltd. (former Mykrolis Corporation), or Kits Microfilter Corporation.

A second filter may be formed of the same material as that of the first filter.

For example, the filtering using the first filter may be performed only on the dispersion, and the second filtering using the second filter may be performed on a mixture of the dispersion and other components.

<Cured Film>

Next, a cured film according to the present invention will be described. The cured film according to the present invention is formed by curing the above-described composition according to the present invention. The cured film according to the present invention has excellent infrared shielding properties and visible transparency and thus can be preferably used as an infrared cut filter.

The cured film according to the present invention may be a film having a pattern or a film (flat film) not having a pattern.

The thickness of the cured film according to the present invention can be adjusted according to the purpose. The thickness is preferably 20 μm or less, more preferably 10 μm or less, and still more preferably 5 μm or less. For example, the lower limit of the thickness is preferably 0.1 μm or more, more preferably 0.2 μm or more, and still more preferably 0.3 μm or more. The cured film according to the present invention can be preferably used as an infrared cut filter of a solid image pickup element such as a charge coupled device (CCD) or a complementary metal-oxide semiconductor (CMOS). In addition, the infrared cut filter according to the present invention can be used in various image display devices.

The cured film (infrared cut filter) according to the present invention can be used in combination with a color filter that includes a chromatic colorant.

The color filter can be manufactured using a coloring composition including a chromatic colorant. Examples of the chromatic colorant include the chromatic colorants described regarding the composition according to the present invention. The coloring composition may further include, for example, a resin, a compound having a crosslinking group, a photopolymerization initiator, a surfactant, a solvent, a polymerization inhibitor, and an ultraviolet absorber. In more detail, for example, the materials described above regarding the infrared absorbing composition according to the present invention can be used. In addition, the cured film (infrared cut filter) according to the present invention may have not only a function as an infrared cut filter but also a function as a color filter by including a chromatic colorant.

In the present invention, “infrared cut filter” refers to a filter that allows transmission of light (visible light) in the visible range and shields light (infrared light) in the infrared range. The infrared cut filter may be a filter that allows transmission of light in the entire wavelength range of the visible range, or may be a filter that allows transmission of light in a specific wavelength range of the visible range and shields light in another specific wavelength range of the visible range.

In addition, in the present invention, “color filter” refers to a filter that allows transmission of light in a specific wavelength range of the visible range and shields light in another specific wavelength range of the visible range.

The cured film (infrared cut filter) according to the present invention may be used in combination with another infrared cut filter other than the cured film according to the present invention. Examples of the other infrared cut filter include a transparent layer containing copper and a band pass filter.

As the transparent layer containing copper, a glass substrate (copper-containing glass substrate) formed of glass containing copper, or a layer (copper complex-containing layer) containing a copper complex may also be used. In addition, in a case where the copper complex-containing layer is used as the transparent layer containing copper, the copper complex-containing layer may be used alone, or may be used in combination with a support.

Examples of the band pass filter include a laminate in which a high refractive index layer and a low refractive index layer are alternately laminated. The spectral characteristics of the band pass filter can be appropriately selected depending on the wavelength of a light source, the spectral characteristics of the infrared cut filter, and the like. By using the cured film in combination with the band pass filter, a wide range of infrared light can also be shielded.

In addition, the infrared cut filter according to the present invention can be used in combination with an infrared transmitting filter. By using an infrared cut filter and an infrared transmitting filter in combination with an infrared transmitting filter, this combination can be preferably used for an infrared sensor that detects infrared light at a specific wavelength. In the present invention, “infrared transmitting filter” refers to a filter that shields light (visible light) in the visible range and allows transmission of light (infrared light) in the infrared range. The wavelength of infrared light that transmits through the infrared transmitting filter can be appropriately selected according to the use.

The infrared cut filter according to the present invention may be or may not be adjacent to the color filter in the thickness direction. In a case where the infrared cut filter is not adjacent to the color filter in the thickness direction, the infrared cut filter may be formed on another substrate other than a substrate on which the color filter is formed, or another member (for example, a microlens or a planarizing layer) constituting a solid image pickup element may be interposed between the infrared cut filter and the color filter.

<Pattern Forming Method>

A pattern forming method according to the present invention includes: a step of forming a composition layer on a support using the composition according to the present invention; and a step of forming a pattern on the composition layer using a photolithography method or a dry etching method.

In a case where a laminate an infrared cut filter and a color filter are laminated is manufactured, pattern formation on the infrared cut filter and pattern formation on the color filter may be separately performed. In addition, pattern formation may be performed on the laminate in which the infrared cut filter and the color filter are laminated (that is, pattern formation on the infrared cut filter and pattern formation on the color filter may be simultaneously performed).

The case where pattern formation on the infrared cut filter and pattern formation on the color filter are separately performed denotes the following aspect. Pattern formation is performed on any one of the infrared cut filter and the color filter. Next, the other filter layer is formed on the filter layer on which the pattern is formed. Next, pattern formation is performed on the filter layer on which a pattern is not formed.

A pattern forming method may be a pattern forming method using photolithography or a pattern forming method using dry etching.

In a case where the pattern forming method using photolithography is adopted, a dry etching step is not necessary, and an effect that the number of steps can be reduced can be obtained.

In a case where the pattern forming method using dry etching is adopted, a photolithography function is not necessary. Therefore, an effect that the concentration of the infrared absorber in the infrared absorbing composition can increase can be obtained.

In a case where the pattern formation on the infrared cut filter and the pattern formation on the color filter are separately performed, the pattern formations on the respective filter layers may be performed using only the photolithography method or only the dry etching method. In addition, after performing the pattern formation on one filter layer using the photolithography method, the pattern formation may be performed on the other filter layer using the dry etching method. In a case where the pattern formation is performed using a combination of the dry etching method and the photolithography method, it is preferable that a pattern is formed on a first layer using the dry etching method and a pattern is formed on a second or subsequent layer using the photolithography method.

It is preferable that the pattern formation using the photolithography method includes: a step of forming a composition layer on a support using each composition; a step of exposing the composition layer in a pattern shape; and a step of forming a pattern by removing a non-exposed portion by development. Optionally, the pattern formation further includes: a step (pre-baking step) of baking the composition layer; and a step (post-baking step) of baking the developed pattern.

In addition, It is preferable that the pattern formation using the dry etching method includes: a step of forming a composition layer on a support using each composition and curing the cured composition layer; a step of forming a photoresist layer on the cured composition layer; a step of obtaining a resist pattern by patterning the photoresist layer by exposure and development; and a step of forming a pattern by dry-etching the cured composition layer by using the resist pattern as an etching mask. Hereinafter, the respective steps will be described.

<<Step of Forming Composition Layer>>

In the step of forming a composition layer, a composition layer is formed on a support using each of the compositions.

As the support, for example, a substrate for a solid image pickup element obtained by providing a solid image pickup element (light-receiving element) such as CCD or CMOS on a substrate (for example, a silicon substrate) can be used.

The pattern according to the present invention may be formed on a solid image pickup element-formed surface (front surface) of the substrate for a solid image pickup element, or may be formed on a solid image pickup element non-formed surface (back surface) thereof.

Optionally, an undercoat layer may be provided on the support to improve adhesion with a layer above the support, to prevent diffusion of materials, or to make a surface of the substrate flat.

As a method of applying the composition to the support, various methods such as slit coating, an ink jet method, spin coating, cast coating, roll coating, or screen printing can be used.

The composition layer formed on the support may be dried (pre-baked). In a case where a pattern is formed through a low-temperature process, pre-baking is not necessarily performed.

In a case where pre-baking is performed, the pre-baking temperature is preferably 150° C. or lower, more preferably 120° C. or lower, and still more preferably 110° C. or lower. The lower limit is, for example, 50° C. or higher or 80° C. or higher. By setting the pre-baking temperature to be 150° C. or lower, the characteristics can be effectively maintained, for example, even in a case where a photoelectric conversion film of an image sensor is formed of an organic material.

The pre-baking time is preferably 10 to 300 seconds, more preferably 40 to 250 seconds, and still more preferably 80 to 220 seconds. Drying can be performed using a hot plate, an oven, or the like.

In a case where the pattern formation is simultaneously performed on a plurality of layers, it is preferable that a composition for forming each of the layers is applied to the composition layer to form another composition layer.

(Case where Pattern is Formed Using Photolithography Method)

### <<Exposure Step>>

Next, the composition layer is exposed in a pattern shape (exposure step). For example, the composition layer is exposed in a pattern shape using an exposure device such as a stepper through a mask having a predetermined mask pattern, thereby exposing a pattern. As a result, an exposed portion can be cured.

As radiation (light) used during the exposure, ultraviolet rays such as g-rays or i-rays are preferably used (i-rays are more preferably used). The irradiation dose (exposure dose) is preferably 0.03 to 2.5 J/cm², more preferably 0.05 to 1.0 J/cm², and most preferably 0.08 to 0.5 J/cm².

The oxygen concentration during exposure can be appropriately selected. The exposure may be performed not only in air but also in a low-oxygen atmosphere having an oxygen concentration of 19 vol % or lower (for example, 15 vol %, 5 vol %, or substantially 0 vol %) or in a high-oxygen atmosphere having an oxygen concentration of higher than 21 vol % (for example, 22 vol %, 30 vol %, or 50 vol %). In addition, the exposure illuminance can be appropriately set and typically can be selected in a range of 1000 W/m² to 100000 W/m² (for example, 5000 W/m², 15000 W/m², or 35000 W/m²). Conditions of the oxygen concentration and conditions of the exposure illuminance may be appropriately combined. For example, conditions are oxygen concentration: 10 vol % and illuminance: 10000 W/m², or oxygen concentration: 35 vol % and illuminance: 20000 W/m².

<<Development Step>>

Next, a pattern is formed by removing a non-exposed portion by development. The non-exposed portion can be removed by development using a developer. As a result, a non-exposed portion of the composition layer in the exposure step is eluted into the developer, and only the photocured portion remains.

As the developer, an organic alkali developer which does not cause damages to a solid image pickup element as a substrate, a circuit or the like is desired.

For example, the temperature of the developer is preferably 20° C. to 30° C. The developing time is preferably 20 to 180 seconds. In addition, in order to further improve residue removing properties, a step of shaking the developer off per 60 seconds and supplying a new developer may be repeated multiple times.

Examples of an alkaline agent used in the developer include an organic alkaline compound such as ammonia water, ethylamine, diethylamine, dimethylethanolamine, tetramethylammonium hydroxide, tetraethylammonium hydroxide, tetrapropylammonium hydroxide, tetrabutylammonium hydroxide, benzyltrimethylammonium hydroxide, choline, pyrrole, piperidine, or 1,8-diazabicyclo-[5.4.0]-7-undecene. As the developer, an alkaline aqueous solution is preferably used in which the above alkaline agent is diluted with pure water such that a concentration thereof is 0.001 to 10 mass % and preferably 0.01 to 1 mass %.

In addition, an inorganic alkali may be used as the developer. Preferable examples of the inorganic alkali include sodium hydroxide, potassium hydroxide, sodium carbonate, sodium bicarbonate, sodium silicate, and sodium metasilicate.

In addition, a surfactant may be added to the developer. Examples of the surfactant include the surfactants described above regarding the composition. Among these, a nonionic surfactant is preferable.

In a case where a developer including the alkaline aqueous solution is used, in general, it is preferable that the film is rinsed with pure water after development.

After the development, it is preferable that the film is dried and then heated (post-baking). Post-baking is a heat treatment which is performed after development to completely cure the film. In a case where post-baking is performed, for example, the post-baking temperature is preferably 100° C. to 240° C. From the viewpoint of curing the film, the post-baking temperature is more preferably 200° C. to 230° C. In addition, in a case where an organic electroluminescence (organic EL) element is used as a light-emitting light source, or in a case where a photoelectric conversion film of an image sensor is formed of an organic material, the post-baking temperature is preferably 150° C. or lower, more preferably 120° C. or lower, still more preferably 100° C. or lower, and even still more preferably 90° C. or lower. The lower limit is, for example, 50° C. or higher.

The film after the development is post-baked continuously or batchwise using heating means such as a hot plate, a convection oven (hot air circulation dryer), a high-frequency heater under the above-described conditions. In addition, in a case where a pattern is formed through a low-temperature process, post-baking is not necessarily performed.

(Case where Pattern is Formed Using Dry Etching Method)

The pattern formation using the dry etching method can be performed by curing the composition layer formed on the support to form a cured composition layer, and then etching the cured composition layer with etching gas by using a patterned photoresist layer as a mask.

Specifically, it is preferable that a positive type or negative type radiation sensitive composition is applied to the cured composition layer and is dried such that a photoresist layer is formed. It is preferable that pre-baking is further performed in order to form the photoresist layer. In particular, in a preferable aspect, as a process of forming the photoresist, baking after exposure or baking after development (post-baking) is performed. The details of the pattern formation using the dry etching method can be found in paragraphs “0010” to “0067” of JP2013-064993A, the content of which is incorporated herein by reference.

<Solid Image Pickup Element>

A solid image pickup element according to the present invention includes the cured film according to the present invention. The solid image pickup element according to the present invention is configured to include the cured film (infrared cut filter) according to the present invention. The configuration of the solid image pickup element is not particularly limited as long as the solid image pickup element functions. For example, the following configuration can be adopted.

The solid image pickup element includes plural photodiodes and transfer electrodes on the support, the photodiodes constituting a light receiving area of the solid image pickup element, and the transfer electrode being formed of polysilicon or the like. In the solid image pickup element, a light shielding film formed of tungsten or the like which has openings through only light receiving sections of the photodiodes is provided on the photodiodes and the transfer electrodes, a device protective film formed of silicon nitride or the like is formed on the light shielding film so as to cover the entire surface of the light shielding film and the light receiving sections of the photodiodes, and the film according to the present invention is formed on the device protective film.

Further, a configuration in which light collecting means (for example, a microlens; hereinafter, the same shall be applied) is provided above the device protective film and below the infrared cut filter according to the present invention (on a side thereof close the support), or a configuration in which light collecting means is provided on the cured film (infrared cut filter) according to the present invention may be adopted.

<Image Display Device>

The cured film according to the present invention can also be used in an image display device such as a liquid crystal display device or an organic electroluminescence (organic EL) display device. For example, by using the cured film in combination with the respective colored pixels (for example, red, green, blue), the near infrared cut filter can be used for the purpose of shielding infrared light included in light emitted from a backlight (for example, a white light emitting diode (white LED)) of a display device to prevent a malfunction of a peripheral device, or for the purpose of forming a pixel of the infrared cut filter in addition to the respective color display pixels.

The definition of a display device and the details of each display device can be found in, for example, “Electronic Display Device (by Akiya Sasaki, Kogyo Chosakai Publishing Co., Ltd., 1990)” or “Display Device (Sumiaki Ibuki, Sangyo Tosho Co., Ltd.). In addition, the details of a liquid crystal display device can be found in, for example, “Next-Generation Liquid Crystal Display Techniques (Edited by Tatsuo Uchida, Kogyo Chosakai Publishing Co., Ltd., 1994)”. The liquid crystal display device to which the present invention is applicable is not particularly limited. For example, the present invention is applicable to various liquid crystal display devices descried in “Next-Generation Liquid Crystal Display Techniques”.

The image display device may include a white organic EL element. It is preferable that the white organic EL element has a tandem structure. The tandem structure of the organic EL element is described in, for example, JP2003-45676A, or pp. 326-328 of “Forefront of Organic EL Technology Development-Know-How Collection of High Brightness, High Precision, and Long Life” (Technical Information Institute, 2008). It is preferable that a spectrum of white light emitted from the organic EL element has high maximum emission peaks in a blue range (430 nm to 485 nm), a green range (530 nm to 580 nm), and a yellow range (580 nm to 620 nm). It is more preferable that the spectrum has a maximum emission peak in a red range (650 nm to 700 nm) in addition to the above-described emission peaks.

<Infrared Absorber, Compound>

Next, a compound according to the present invention will be described.

The compound according to the present invention is a compound (squarylium compound) represented by Formula (1A) described above regarding the composition according to the present invention, and a preferable range thereof is also as described above.

The compound according to the present invention can be preferably used as an infrared absorber.

The compound according to the present invention can be preferably used to form, for example, an infrared cut filter that shields light in a wavelength range of 700 to 1000 nm. In addition, the compound according to the present invention can be used as an infrared cut filter for a plasma display panel or a solid image pickup element, an optical filter such as a heat ray shielding film, or a photothermal conversion material such as a recordable optical disc (CD-R) or a flash melt fixing material. In addition, the compound according to the present invention can be used as an information display material for security ink or invisible barcode ink.

### EXAMPLES

Hereinafter, the present invention will be described in detail using examples. Materials, used amounts, ratios, treatment details, treatment procedures, and the like shown in the following examples can be appropriately changed within a range not departing from the scope of the present invention. Accordingly, the scope of the present invention is not limited to the following specific examples. Unless specified otherwise, “part(s)” and “%” represent “part(s) by mass” and “mass %”.

A structure of a compound used as an infrared absorber is the same as that the compound having the chemical structure described above regarding the infrared absorber.

<Measurement of Weight-Average Molecular Weight (Mw)>

The weight-average molecular weight (Mw) was measured using the following method.

Kind of Column: TSKgel Super HZ4000 (manufactured by Tosoh Corporation, 4.6 mm (Inner diameter)×15 cm)

Developing solvent: tetrahydrofuran Column temperature: 40° C.

Flow rate (sample injection volume): 60 μL

Device name: High-Speed GPC (HLC-8220GPC), manufactured by Tosoh Corporation

Calibration curve base resin: polystyrene

<Synthesis of Compound>

### Synthesis Example 1

A compound SQ13 was synthesized according to the following synthesis scheme.

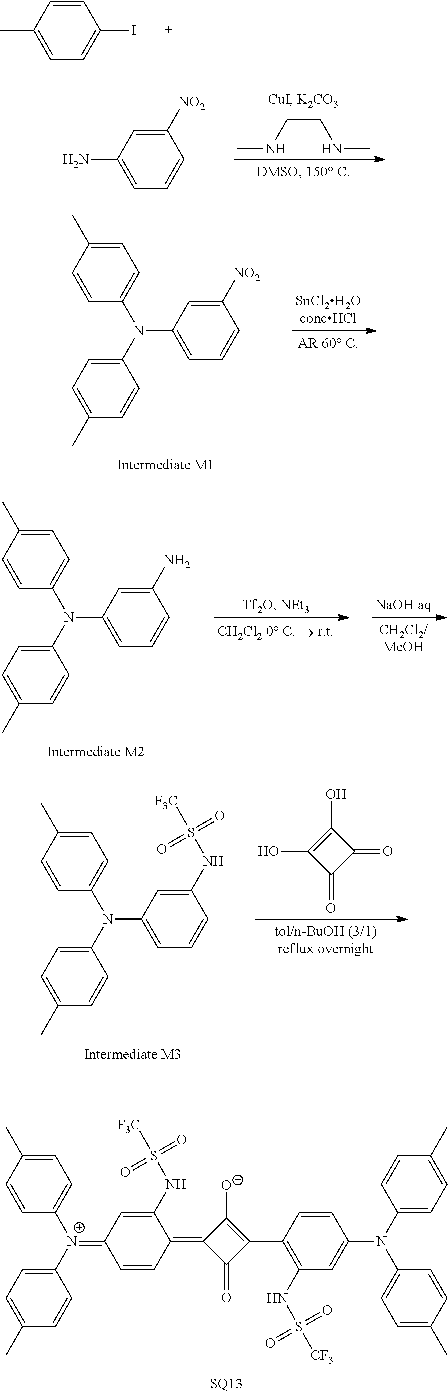

An intermediate M1 and an intermediate M2 were synthesized using a method described in Tetrahedron Lett. 1996, 37, 9207-9210 according to the scheme shown above. An intermediate M3 was synthesized by treating the intermediate M2 using a method described in Tetrahedron Lett. 2008, 49, 6300-6303 to form monosulfonamide according to the scheme shown above.

The intermediate M3 (3.1 g, 7.5 mmol) and squaric acid (0.43 g, 3.7 mmol) were heated to reflux for 12 hours under azeotropic dehydration in n-butanol/toluene (5.2 cm³/16.0 cm³). The reaction solution was cooled, the solvent was removed by distillation under reduced pressure, and the residue was purified by silica gel column chromatography (developing solvent: chloroform). Chloroform was removed by distillation under reduced pressure, the solid was ultrasonically dispersed in methanol, and the solid was filtered under reduced pressure. As a result, a target compound (compound SQ13) was obtained (green crystal, 1.2 g, yield: 37%).

Identification data of the compound SQ13: MALDI time-of-flight mass spectrometry (TOF-MASS)

Calc. for [M+H]+: 919.2, found: 919.2.

### Synthesis Example 2

A compound SQ14 was synthesized according to the following synthesis scheme.

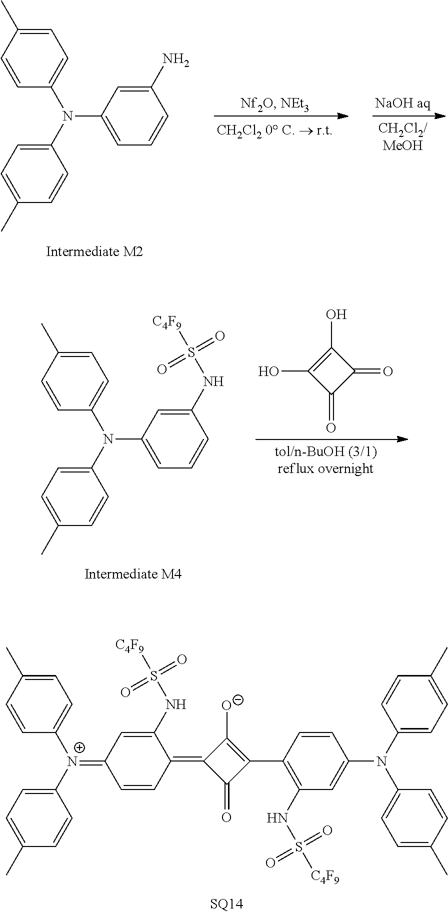

An intermediate M4 was synthesized using the same method as that of the intermediate M3, except that nonafluorobutanesulfonic anhydride was used instead of trifluoromethanesulfonic anhydride.

### (Synthesis of Compound SQ14)

A compound SQ14 was synthesized using the same synthesis method as that of the compound SQ13, except that the intermediate M4 was used instead of the intermediate M3.

Identification data of the compound SQ14: MALDI time-of-flight mass spectrometry (TOF-MASS)

Calc. for [M+H]+: 1219.2, found: 1219.3.

### Synthesis Example 3

A compound SQ56 was synthesized according to the following synthesis scheme.

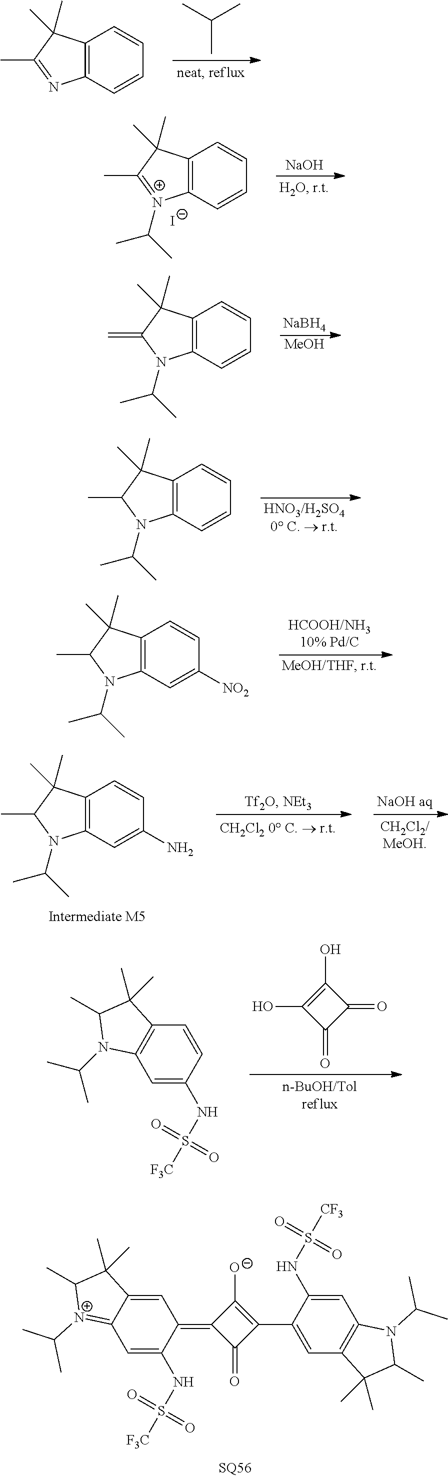

An intermediate M5 was synthesized using a method described in WO2014/088063A1 according to the scheme shown above.

An intermediate M6 was synthesized using the same synthesis method as that of the intermediate M3, except that the intermediate M5 was used instead of the intermediate M2.

A compound SQ56 was synthesized using the same synthesis method as that of the compound SQ13, except that the intermediate M6 was used instead of the intermediate M3.

Identification data of the compound SQ56: MALDI time-of-flight mass spectrometry (TOF-MASS)

Calc. for [M+H]+: 779.2, found: 779.2.

### Synthesis Example 4

A compound SQ49 was synthesized according to the following synthesis scheme.

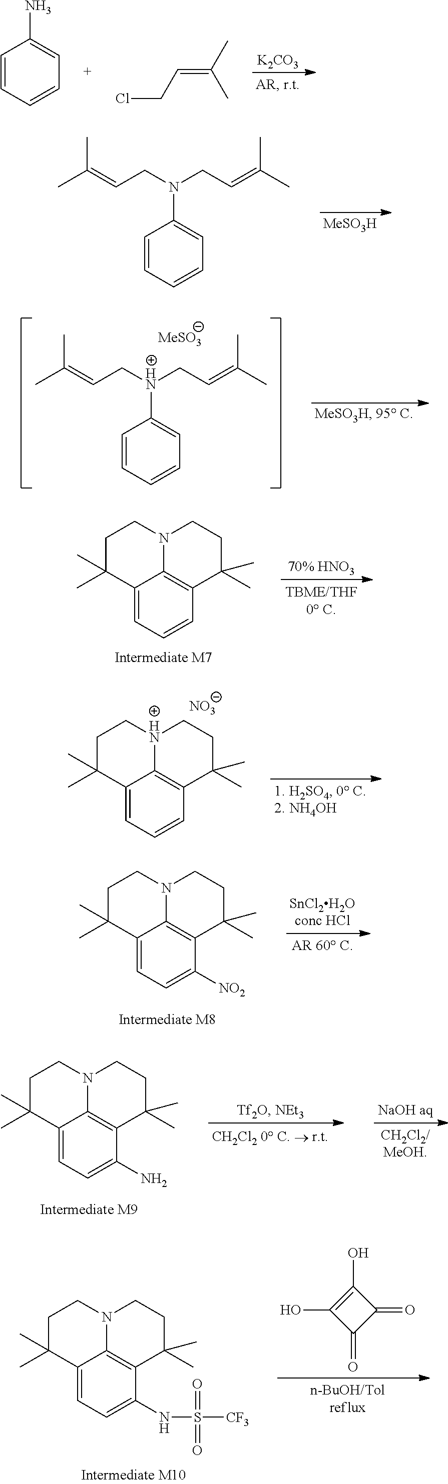

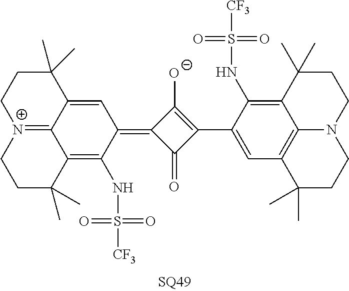 [SMILES: CC1(C)CCN2CCC(C)(C)c3c(NS(=O)(=O)C(F)(F)F)c(C4=C([O-])/C(=c5\cc6c7c(c5NS(=O)(=O)C(F)(F)F)C(C)(C)CC[N+]=7CCC6(C)C)C4=O)cc1c32]

An intermediate M7 was synthesized using a method described in Tetrahedron Letters 44 (2003), 145-147 according to the scheme shown above. An intermediate M8 was synthesized by nitrating the intermediate 7 using a method described in Tetrahedron Letters 48 (2007), 8659-8664 according to the scheme shown above. An intermediate M9 was synthesized using the same synthesis method as that of the intermediate M3, except that the intermediate M8 was used instead of the intermediate M2. An intermediate M10 was synthesized using the same synthesis method as that of the intermediate M2, except that the intermediate M8 was used instead of the intermediate M1.

A compound SQ49 was synthesized using the same synthesis method as that of the compound SQ13, except that the intermediate M10 was used instead of the intermediate M3.

Identification data of the compound SQ49: MALDI time-of-flight mass spectrometry (TOF-MASS)

Calc. for [M+H]+: 831.3, found: 831.2.

### Synthesis Example 5

A compound SQ10 was synthesized according to the following synthesis scheme.

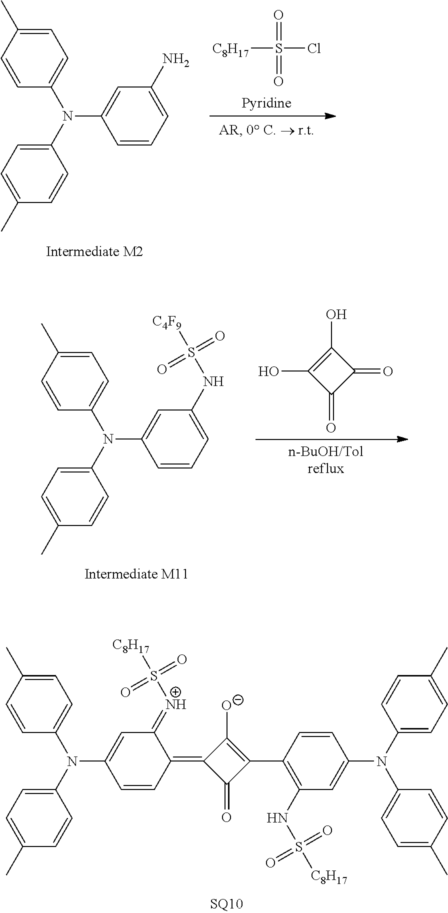

The intermediate M2 (2.5 g, 8.5 mmol) and pyridine (0.81 g, 10.2 mmol) were dissolved in 20 ml of acetonitrile, and octylsulfonic acid chloride was added dropwise thereto at 0° C. The reaction solution was stirred at room temperature for 4 hours, and water was added for quenching, and a target compound was extracted with ethyl acetate. The obtained oil layer was rinsed with water three times, the oil layer was dried with magnesium sulfate, and ethyl acetate was removed by distillation under reduced pressure. The residue was purified by silica gel column chromatography (developing solvent: hexane/ethyl acetate=5/1). As a result, an intermediate M11 was obtained (colorless liquid, 2.7 g, yield: 70%).

A compound SQ10 was synthesized using the same synthesis method as that of the compound SQ13, except that the intermediate M11 was used instead of the intermediate M3.

Identification data of the compound SQ10: MALDI time-of-flight mass spectrometry (TOF-MASS)

Calc. for [M+H]+: 1007.5, found: 1007.5.

### Synthesis Example 6

A compound SQ3 was synthesized according to the following synthesis scheme.

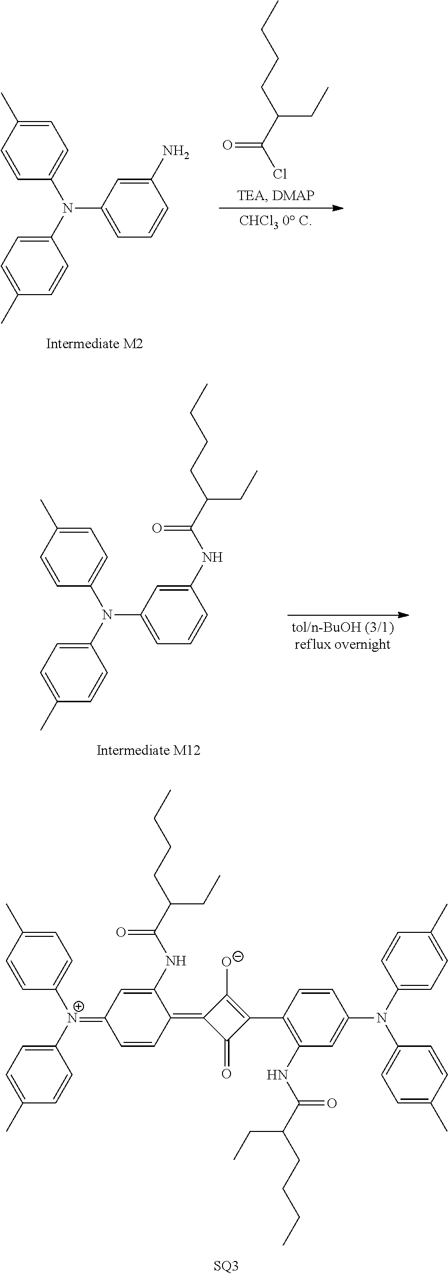

The intermediate M2 (3.6 g, 12.6 mmol), triethylamine (2.6 g, 25.2 mmol), and a catalytic amount of N,N-dimethylaminopyridine were dissolved in 63 ml of chloroform, and 2-ethylhexanoic acid chloride (3.1 g, 18.9 mmol) was added dropwise at 0° C. The reaction solution was stirred at 0° C. for 1 hour, and water was added for quenching, and a target compound was extracted with chloroform. The obtained oil layer was rinsed with water two times, the oil layer was dried with magnesium sulfate, and chloroform was removed by distillation under reduced pressure. The residue was purified by column chromatography (developing solvent: hexane/ethyl acetate=7/1). As a result, an intermediate M12 was obtained (colorless liquid, 3.9 g, yield: 75%).

A compound SQ3 was synthesized using the same synthesis method as that of the compound SQ13, except that the intermediate M12 was used instead of the intermediate M3.

Identification data of the compound SQ3: MALDI time-of-flight mass spectrometry (TOF-MASS)

Calc. for [M+H]+: 907.5, found: 907.5.

### Synthesis Example 7

A compound SQ52 was synthesized according to the following synthesis scheme.

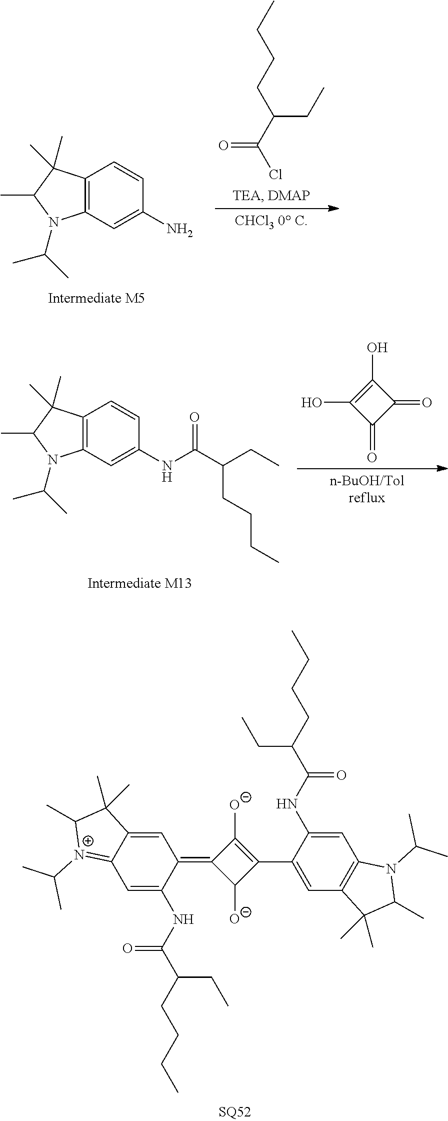

An intermediate M13 was synthesized using the same synthesis method as that of the intermediate M12, except that the intermediate M5 was used instead of the intermediate M2.

A compound SQ52 was synthesized using the same synthesis method as that of the compound SQ13, except that the intermediate M13 was used instead of the intermediate M3.

Identification data of the compound SQ52: MALDI time-of-flight mass spectrometry (TOF-MASS)

Calc. for [M+H]+: 768.5, found: 768.4.

### Synthesis Example 8

A compound SQ41 was synthesized according to the following synthesis scheme.

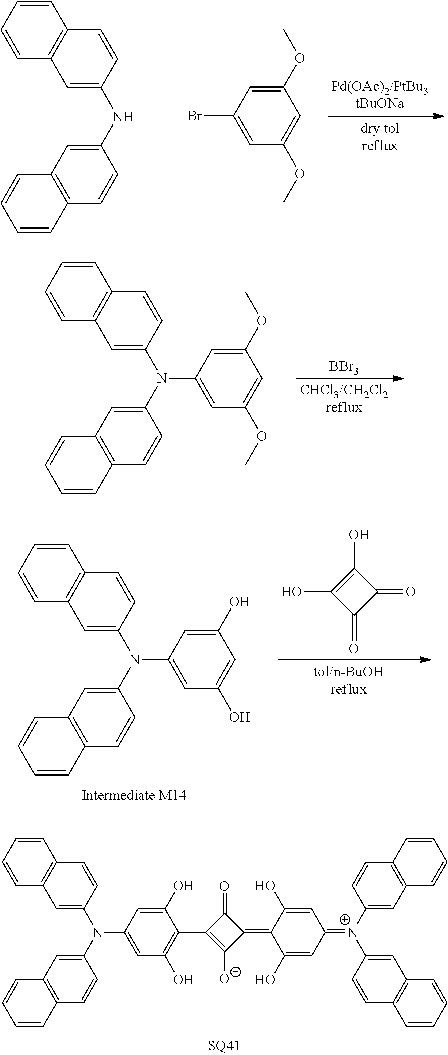

An intermediate M14 was synthesized using a method described in Chem. Mater. 2011, 23, 4789-4798 according to the scheme shown above.

A compound SQ41 was synthesized using the same synthesis method as that of the compound SQ13, except that the intermediate M14 was used instead of the intermediate M3.

Identification data of the compound SQ41: MALDI time-of-flight mass spectrometry (TOF-MASS)

Calc. for [M+H]+: 833.3, found: 833.3.

### Synthesis Example 9

A compound SQ66 was synthesized according to the following synthesis scheme.

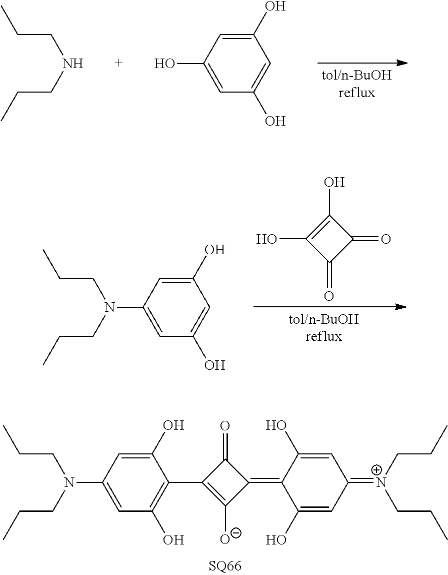

A compound SQ66 was synthesized using a method described in J. Phys. Chem. B, 2002, 106, 4370-4376 according to the scheme shown above.

Identification data of the compound SQ66: MALDI time-of-flight mass spectrometry (TOF-MASS)

Calc. for [M+H]+: 496.3, found: 497.2.

### Synthesis Example 10

A compound SQ93 was synthesized according to the following synthesis scheme.

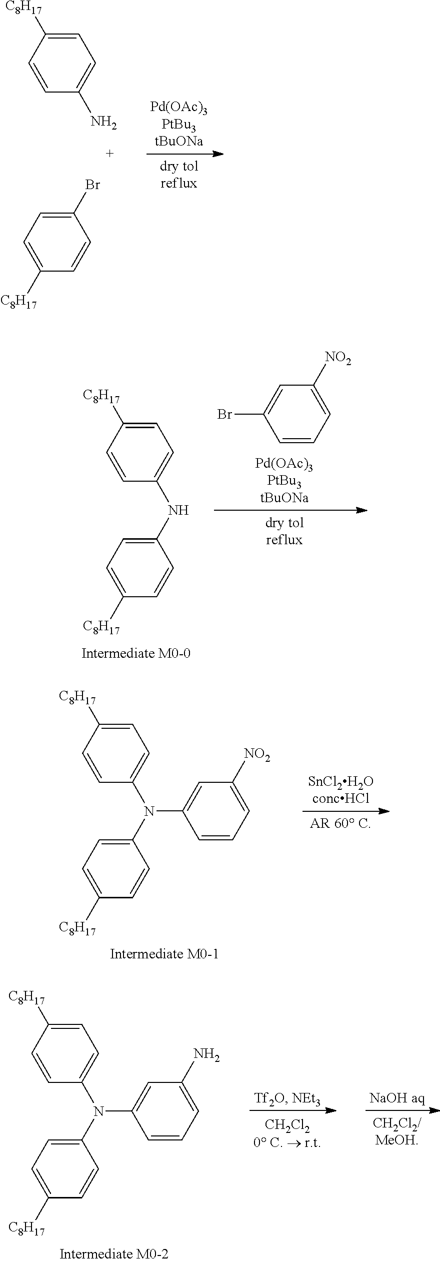

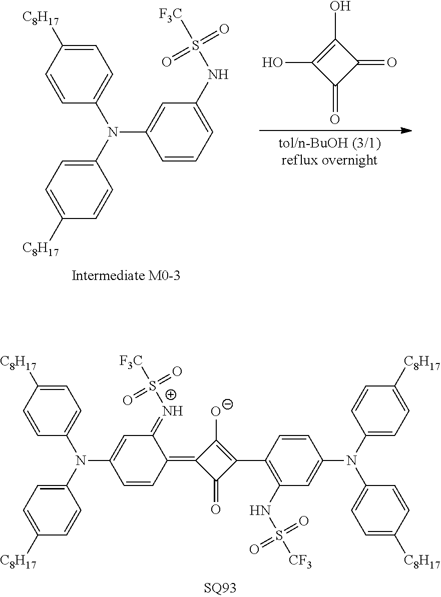

An intermediate M0-0 was synthesized using a method described in J. AM. Chem. Soc. 2010, 132, 7478-7487 according to the scheme shown above.

An intermediate M0-1 was synthesized using a method described in WO2012/121936A according to the scheme shown above.

An intermediate M0-2 was synthesized using a method described in Tetrahedron Lett. 1996, 37, 9207-9210 according to the scheme shown above.

An intermediate M0-3 was synthesized by treating the intermediate M0-2 using a method described in Tetrahedron Lett. 2008, 49, 6300-6303 to form monosulfonamide according to the scheme shown above.

The intermediate M0-3 (4.6 g, 7.5 mmol) and squaric acid (0.43 g, 3.7 mmol) were heated to reflux for 12 hours under azeotropic dehydration in n-butanol/toluene (5.2 cm³/16.0 cm³). The reaction solution was cooled, the solvent was removed by distillation under reduced pressure, and the residue was purified by silica gel column chromatography (developing solvent: chloroform). Chloroform was removed by distillation under reduced pressure, the solid was ultrasonically dispersed in methanol, and the solid was filtered under reduced pressure. As a result, a target compound (compound SQ93) was obtained (green crystal, 1.2 g, yield: 24%).

Identification data of the compound SQ93: MALDI time-of-flight mass spectrometry (TOF-MASS)

Calc. for [M+H]+: 1311.6, found: 1311.7.

### Synthesis Example 11

A compound SQ95 was synthesized according to the following synthesis scheme.

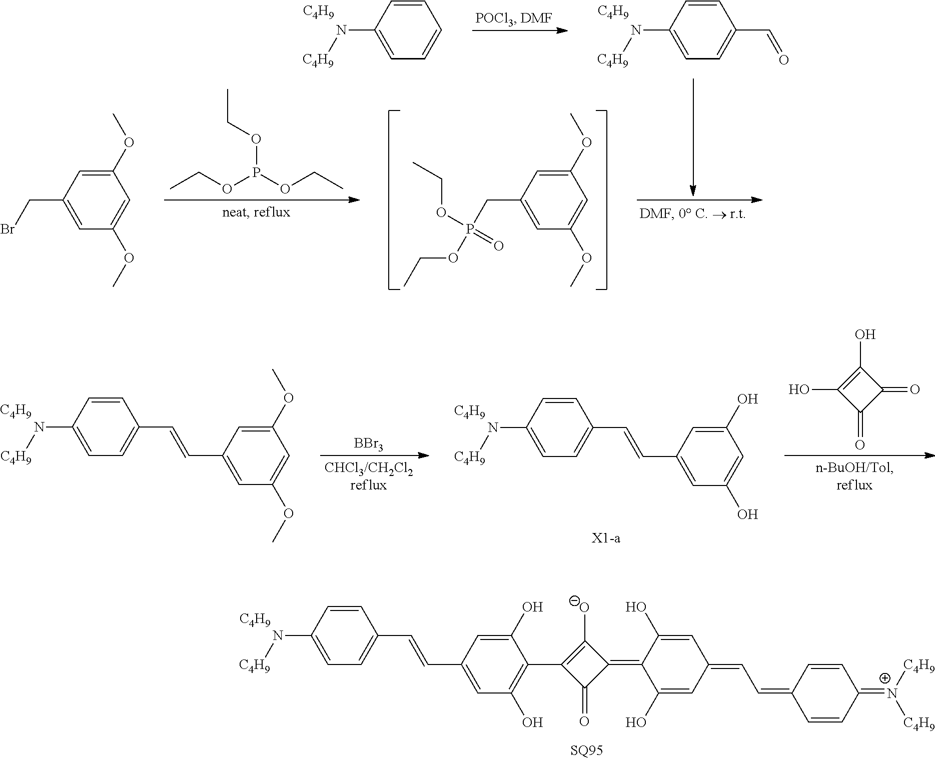

An intermediate X1-a was synthesized using a method described in Chem. Commun. 1999, 997-978 according to the scheme shown above, except that dibutylaminobenzene was used as a raw material.

A compound SQ95 was synthesized using the same synthesis method as that of the compound SQ13, except that the intermediate X1-a was used instead of the intermediate M3.

Identification data of the compound SQ95: MALDI time-of-flight mass spectrometry (TOF-MASS)

Calc. for [M]−: 756.4, found: 756.4.

### Synthesis Example 12

A compound SQ97 was synthesized according to the following synthesis scheme.

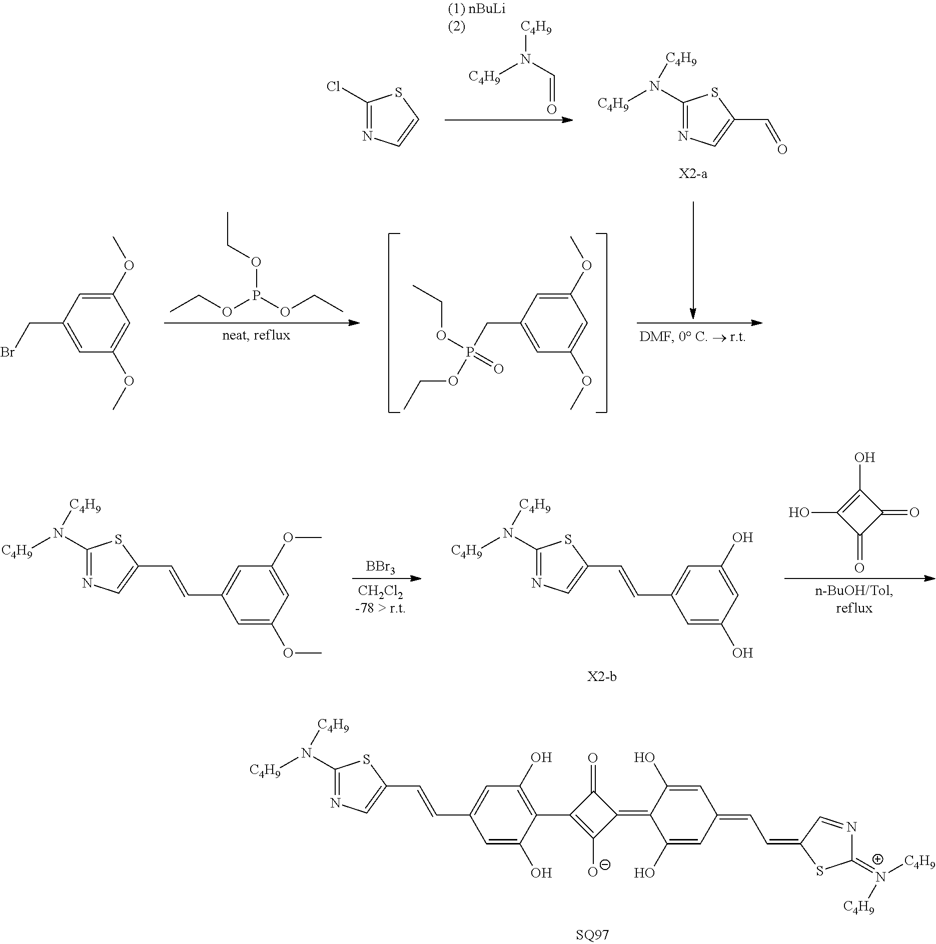

An intermediate X2-a was synthesized using a method described in J. Mater. Chem. 1998, 8, 833-835, except that N,N-dibutylformamide was used as a raw material.

An intermediate X2-b was synthesized using a method described in Helvetica Chemica Acta 2004, 87, 1109-1118 according to the scheme shown above.

A compound SQ97 was synthesized using the same synthesis method as that of the compound SQ13, except that the intermediate X2-b was used instead of the intermediate M3.

Identification data of the compound SQ97: MALDI time-of-flight mass spectrometry (TOF-MASS)

Calc. for [M]−: 770.3, found: 770.3.

<Preparation of Near Infrared Absorbing Curable Composition>

### Examples 1 to 21

Raw materials having the following compositions were mixed with each other to prepare near infrared absorbing curable compositions.

### <Composition 1>

Compound shown in Table 1: 2.3 parts

Resin 1: 12.9 parts

Crosslinking compound 1: 12.9 parts

Polymerization initiator 1: 2.5 parts

Ultraviolet absorber 1: 0.5 parts

Surfactant 1: 0.04 parts

Polymerization inhibitor (p-methoxyphenol): 0.006 parts

Cyclohexanone: 49.6 parts

Propylene glycol monomethyl ether acetate: 19.3 parts

### <Composition 2>

Compound shown in Table 1: 2.3 parts

Resin 2: 12.9 parts

Crosslinking compound 1: 12.9 parts

Polymerization initiator 1: 2.5 parts

Ultraviolet absorber 1: 0.5 parts

Surfactant 1: 0.04 parts

Polymerization inhibitor (p-methoxyphenol): 0.006 parts

Cyclohexanone: 49.6 parts

Propylene glycol monomethyl ether acetate: 19.3 parts

### <Composition 3>

Compound shown in Table 1: 2.3 parts

Resin 3: 12.9 parts

Crosslinking compound 2: 12.9 parts

Acid generator 1: 2.5 parts

Ultraviolet absorber 1: 0.5 parts

Surfactant 1: 0.04 parts

Cyclohexanone: 49.6 parts

Propylene glycol monomethyl ether acetate: 19.3 parts

### <Composition 4>

Compound shown in Table 1: 2.3 parts

Crosslinking compound 3 (polymer): 12.9 parts

Acid catalyst (phosphoric acid): 2.5 parts

Ultraviolet absorber 1: UV503 (manufactured by Daito Chemical Co., Ltd.): 0.5 parts

Surfactant 1: the following mixture (Mw:14000): 0.04 parts

Cyclohexanone: 58.9 parts

Propylene glycol monomethyl ether acetate: 22.9 parts

### <Composition 5>

Each of compounds shown in Table 1: 1.2 parts

Resin 2: 12.8 parts

Crosslinking compound 1: 12.9 parts

Polymerization initiator 1: 2.5 parts

Ultraviolet absorber 1: 0.5 parts

Surfactant 1: 0.04 parts

Polymerization inhibitor (p-methoxyphenol): 0.006 parts

Cyclohexanone: 49.6 parts

Propylene glycol monomethyl ether acetate: 19.3 parts

### <Composition 6>

Compound shown in Table 1: 2.3 parts

Resin 4: 12.9 parts

Crosslinking compound 1: 12.8 parts

Polymerization initiator 1: 2.5 parts

Ultraviolet absorber 1: 0.5 parts

Surfactant 1: 0.04 parts

Polymerization inhibitor (p-methoxyphenol): 0.006 parts

Cyclohexanone: 49.6 parts

Propylene glycol monomethyl ether acetate: 19.3 parts

(Resin)
Resin 1: a copolymer including benzyl methacrylate (BzMA) and methacrylic acid (MAA) (composition ratio (mass ratio): (BzMA/MAA)=(80/20), Mw=15000)
Resin 2: a copolymer including allyl methacrylate (AMA) and methacrylic acid (MAA) (composition ratio (mass ratio): (AMA/MAA)=(80/20), Mw=15000)
Resin 3: a copolymer including glycidyl methacrylate (GlyMA) and methacrylic acid (MAA) (composition ratio (mass ratio): (GlyMA/MAA)=(80/20), Mw=15000)
Resin 4: ARTON F4520 (manufactured by JSR Corporation)

### (Crosslinking Compound)

Crosslinking compound 1: dipentaerythritol hexaacrylate (trade name: KAYARAD DPHA, manufactured by Nippon Kayaku Co., Ltd.)

Crosslinking compound 2: OXT-221 (manufactured by Toagosei Co., Ltd.)

Crosslinking compound 3: the following structure (numerical values added to the repeating unit represent a molar ratio)

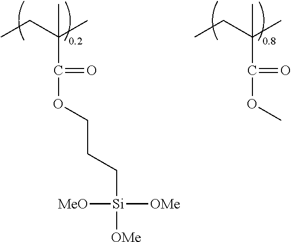

### (Polymerization Initiator)

Polymerization initiator 1: IRGACURE-OXE01 (manufactured by BASF SE, [2-(O-benzoyloxime)-1-[4-(phenylthio)phenyl]-1,2-octanedione]

### (Acid Generator)

Acid generator 1: CPI-100P (manufactured by San-Apro Ltd.)

### (Ultraviolet Absorber)

Ultraviolet absorber 1: UV503 (manufactured by Daito Chemical Co., Ltd.)

### (Surfactant)

Surfactant 1: the following mixture (Mw:14000)

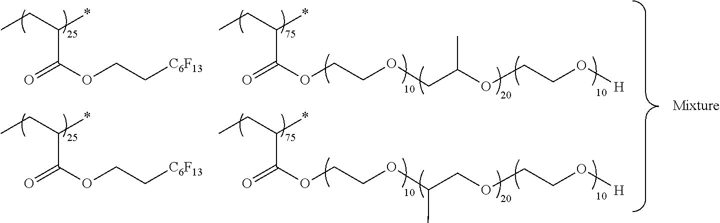

Compounds used in Examples are as follows.

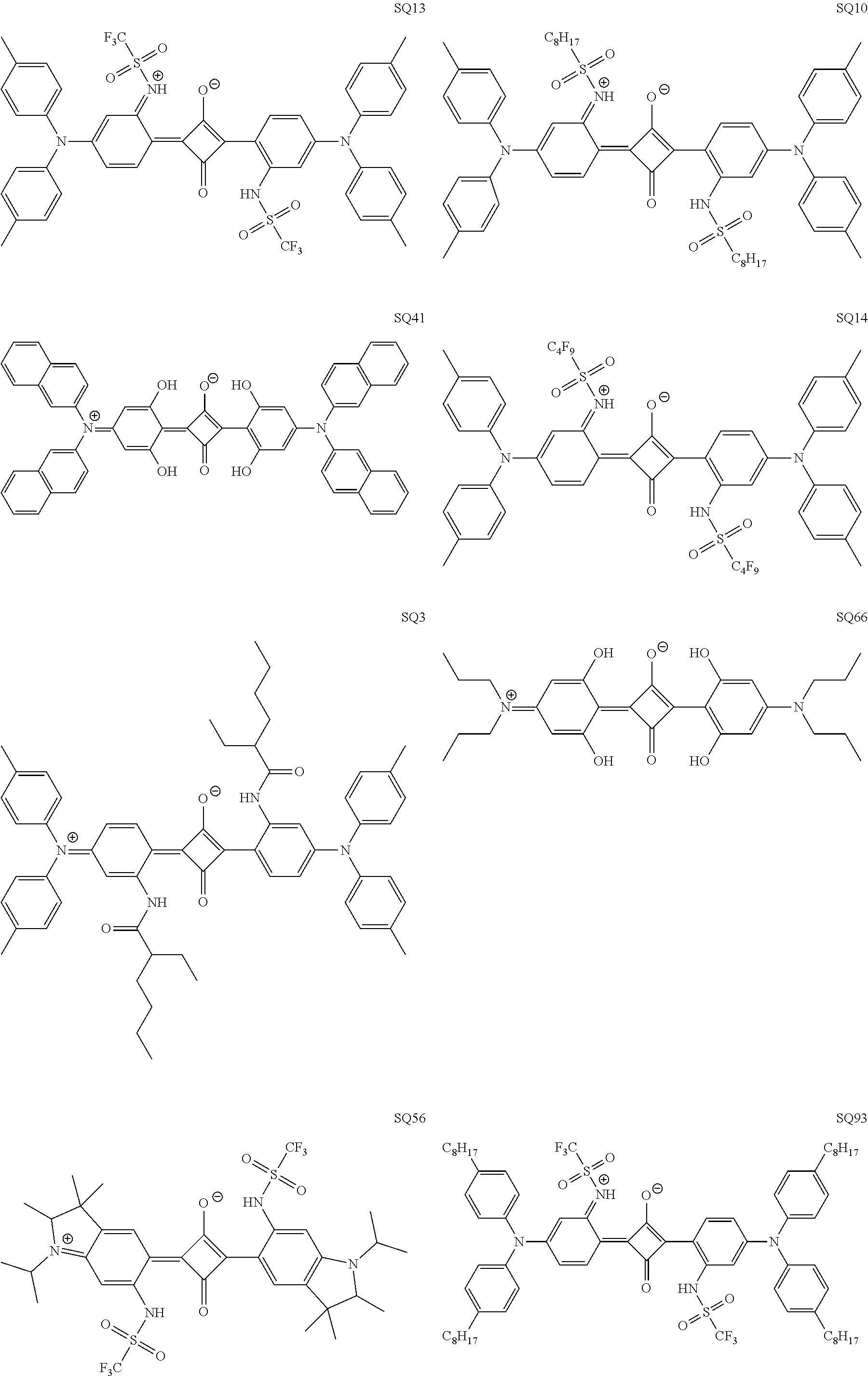

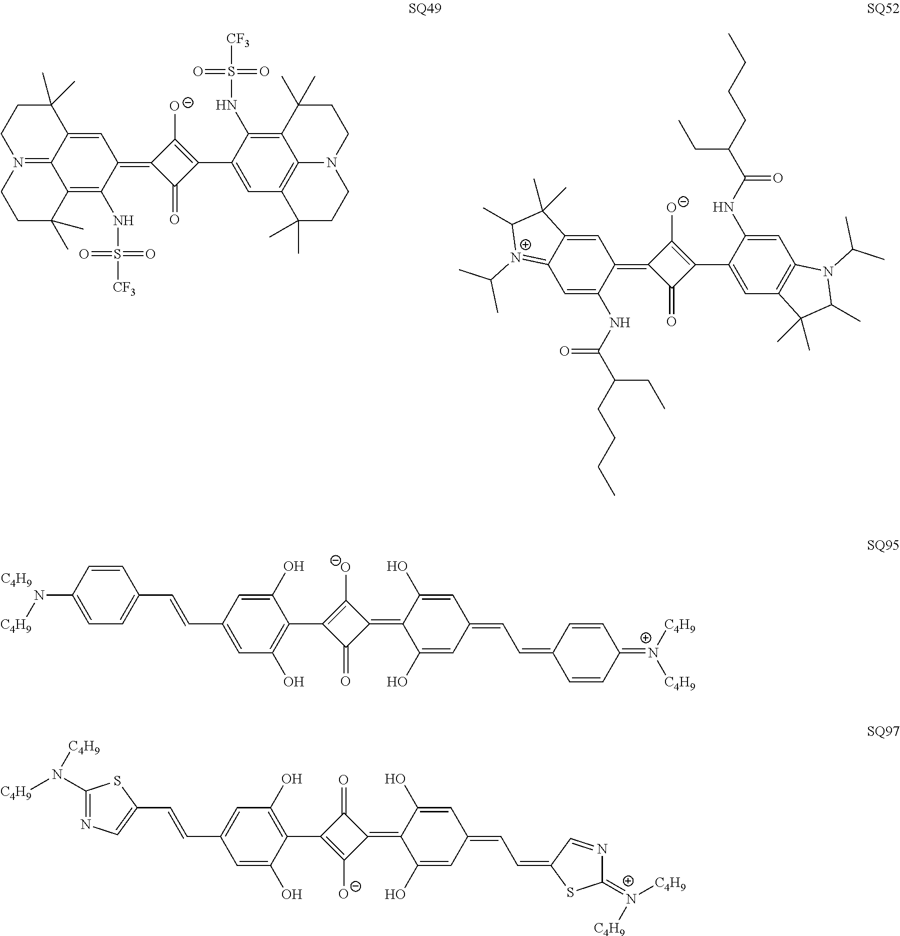

<Preparation of Cured Film>

### Preparation Example 1

### (Method of Preparing Cured Films Using Near Infrared Absorbing Curable Composition Having Compositions 1 to 3, 5, and 6)

Each of the compositions was applied to a glass substrate (1737, manufactured by Corning Inc.) using a spin coater such that the thickness of a dried film was 1.0 μm, and was heated (pre-baked) using a hot plate at 100° C. for 120 seconds.

Next, the entire surface of the glass substrate was exposed using an i-ray stepper exposure device FPA-3000 i5+ (manufactured by Canon Corporation) at 500 mJ/cm². Next, the glass substrate underwent puddle development at 23° C. for 60 seconds using a developing device CD-2060 (manufactured by Fujifilm Electronic Materials Co., Ltd.), was rinsed with pure water, and was spin-dried. Further, the glass substrate was heated (post-baked) using a hot plate at 200° C. for 300 seconds. As a result, a cured film (infrared cut filter) was obtained.

### Preparation Example 2

### (Method of Preparing Cured Film Using Near Infrared Absorbing Curable Composition Having Composition 4)

The composition having the composition 4 was applied to a glass substrate (1737, manufactured by Corning Inc.) using a spin coater such that the thickness of a dried film was 1.0 μm, and was heated (pre-baked) using a hot plate at 100° C. for 120 seconds. Next, the glass substrate was heated (post-baked) using a hot plate at 200° C. for 300 seconds. As a result, a cured film (infrared cut filter) was obtained.

<Absorption Maximum (λmax) of Cured Film>

Using a spectrophotometer UV-3100PC (manufactured by Shimadzu Corporation), the absorption spectrum of the obtained cured film was measured, and the absorption maximum (λmax) of the cured film was measured.

<Evaluation of Near Infrared Shielding Properties>

The transmittance of each of the cured films at a wavelength of 700 nm was measured using a spectrophotometer U-4100 (manufactured by Hitachi High-Technologies Corporation). The near infrared shielding properties were evaluated based on the following standards. The results are shown in the following table.

A: transmittance at a wavelength of 700 nm≤5%

B: 5%<transmittance at a wavelength of 700 nm≤7%

C: 7%<transmittance at a wavelength of 700 nm≤10%

D: 10%<transmittance at a wavelength of 700 nm

<Evaluation of Visible Transparency>

The transmittance of each of the cured films at a wavelength of 450 to 600 nm was measured using a spectrophotometer U-4100 (manufactured by Hitachi High-Technologies Corporation). The visible transparency was evaluated based on the following standards. The results are shown in the following table.

A: 95%≤minimum value of transmittance at a wavelength of 450 to 600 nm

B: 90%≤minimum value of transmittance at a wavelength of 450 to 600 nm<95%

C: 80%≤minimum value of transmittance at a wavelength of 450 to 600 nm<90%

D: minimum value of transmittance at a wavelength of 450 to 600 nm<80%

<Heat Resistance>

The obtained cured film was heated at 200° C. for 5 minutes. A ΔEab value of a color difference before and after a heat resistance test was measured using a colorimeter MCPD-1000 (manufactured by Otsuka Electronics Co., Ltd.). The lower the ΔEab value, the higher the heat resistance.

The ΔEab value was obtained from the following color difference formula of CIE 1976 (L*, a*, b*) color space (Handbook of Color Science, p. 266, 1985, edited by The Color Science Association Of Japan).

ΔEab={(ΔL*)²+(Δa*)²+(Δb*)²}^{1/2}

### <<Determination Standards>>

A: ΔEab value<3

B: 3≤ΔEab value<5

C: 5≤ΔEab value<10

D: 10≤ΔEab value

<Light Fastness>

The obtained cured film was irradiated with light at 10000 lux using a Xe lamp through an ultraviolet cut filter for 10 hours. A ΔEab value of a color difference before and after a light fastness test was measured using a colorimeter MCPD-1000 (manufactured by Otsuka Electronics Co., Ltd.).

### <<Determination Standards>>

A: ΔEab value<3

B: 3≤ΔEab value<5

C: 5≤ΔEab value<10

D: 10≤ΔEab value

<Solvent Resistance>

Each of the cured films was dipped in solvents (propylene glycol monomethyl ether acetate (PGMEA), acetone, and ethanol) shown in the following table for 120 seconds and was dried at 100° C. for 2 minutes. A case where there were no differences in spectral properties, the thickness, and external appearance before and after dipping each of the cured films in the solvents was evaluated as A. A case where there were differences in any one of spectral properties, the thickness, and external appearance before and after dipping each of the cured films in the solvents was evaluated as B.

### Comparative Examples 1 and 2

Raw materials having the following composition 11 were mixed with each other to prepare near infrared absorbing curable compositions. Cured films (infrared cut filters) were obtained using the same method as in Preparation Example 1, except that the obtained near infrared absorbing curable compositions were used. Using the obtained cured films and the same method as in Examples, the near infrared shielding properties, the visible transparency, the heat resistance, the light fastness, and the solvent resistance were evaluated. In addition, the absorption maximum (λmax) of each of the cured films immediately after the preparation was measured.

### <Composition 11>

Compound shown in Table 1: 2.3 parts

Resin 2: 12.9 parts

Crosslinking compound 1: 12.9 parts

Polymerization initiator 1: 2.5 parts

Ultraviolet absorber 1: 0.5 parts

Surfactant 1: 0.04 parts

Polymerization inhibitor (p-methoxyphenol): 0.006 parts

Cyclohexanone: 49.6 parts

Propylene glycol monomethyl ether acetate: 19.3 parts

### Comparative Example 3

Raw materials having the following composition 12 were mixed with each other to prepare a near infrared absorbing curable composition. A film (infrared cut filter) was obtained using the same method as in Preparation Example 1, except that the obtained near infrared absorbing curable composition was used. Using the obtained film and the same method as in Examples, the near infrared shielding properties, the visible transparency, the heat resistance, the light fastness, and the solvent resistance were evaluated. In addition, the absorption maximum (λmax) of the film immediately after the preparation was measured.

### <Composition 12>

Compound shown in Table 1: 2.3 parts

Resin 1: 28.3 parts

Ultraviolet absorber 1: 0.5 parts

Surfactant 1: 0.04 parts

Cyclohexanone: 49.6 parts

Propylene glycol monomethyl ether acetate: 19.3 parts

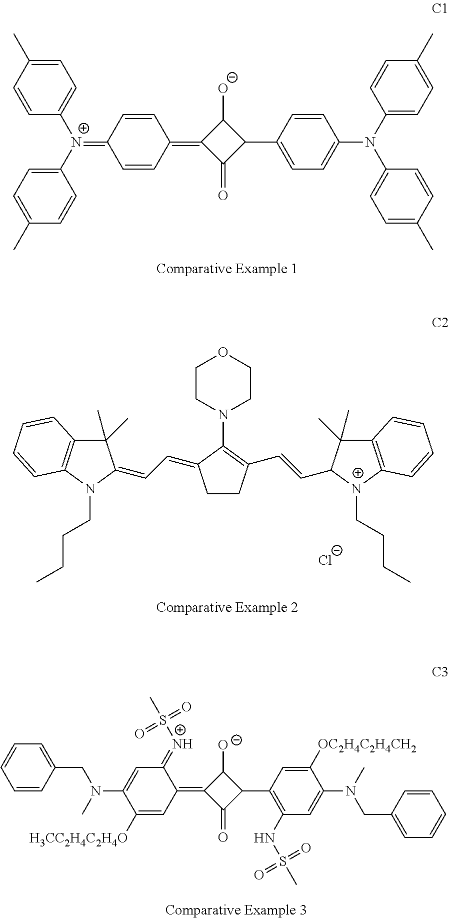

TABLE 1

| | | | Initial Performance | | | | | | | |
|---|---|---|---|---|---|---|---|---|---|---|
| | | | | Infrared Shielding | Visible | Heat | Light | Solvent Resistance | | |
| | Composition | Compound | λmax | Properties | Transparency | Resistance | Fastness | PGMEA | Acetone | Ethanol |
| Example 1 | Composition 1 | SQ93 | 720 nm | A | A | A | A | A | A | A |
| Example 2 | Composition 2 | SQ93 | 720 nm | A | A | A | A | A | A | A |
| Example 3 | Composition 3 | SQ93 | 720 nm | A | A | A | A | A | A | A |
| Example 4 | Composition 4 | SQ93 | 720 nm | A | A | A | A | A | A | A |
| Example 5 | Composition 1 | SQ13 | 720 nm | A | A | A | A | A | A | A |
| Example 6 | Composition 2 | SQ13 | 720 nm | A | A | A | A | A | A | A |
| Example 7 | Composition 3 | SQ13 | 720 nm | A | A | A | A | A | A | A |
| Example 8 | Composition 4 | SQ13 | 720 nm | A | A | A | A | A | A | A |
| Example 9 | Composition 2 | SQ14 | 720 nm | A | A | A | A | A | A | A |
| Example 10 | Composition 2 | SQ56 | 700 nm | A | B | B | B | A | A | A |
| Example 11 | Composition 2 | SQ49 | 690 nm | B | B | B | B | A | A | A |
| Example 12 | Composition 2 | SQ10 | 710 nm | A | C | A | A | A | A | A |
| Example 13 | Composition 2 | SQ3 | 720 nm | A | C | A | A | A | A | A |
| Example 14 | Composition 2 | SQ52 | 725 nm | A | C | B | C | A | A | A |
| Example 15 | Composition 3 | SQ52 | 725 nm | A | C | B | C | A | A | A |
| Example 16 | Composition 6 | SQ52 | 725 nm | A | C | B | C | A | A | A |
| Example 17 | Composition 2 | SQ41 | 690 nm | B | B | A | B | A | A | A |
| Example 18 | Composition 2 | SQ66 | 665 nm | C | C | B | C | A | A | A |
| Example 19 | Composition 2 | SQ95 | 830 nm | C | C | B | B | A | A | A |
| Example 20 | Composition 2 | SQ97 | 775 nm | B | C | B | B | A | A | A |
| Example 21 | Composition 5 | SQ52 + SQ97 | 745 nm | A | C | C | C | A | A | A |
| Comparative | Composition 11 | C1 | 660 nm | D | C | D | D | A | A | A |
| Example 1 | | | | | | | | | | |
| Comparative | Composition 11 | C2 | 675 nm | C | C | D | D | A | A | A |
| Example 2 | | | | | | | | | | |
| Comparative | Composition 12 | C3 | 670 nm | C | C | C | C | B | B | B |
| Example 3 | | | | | | | | | | |

As shown in the table, in Examples, the infrared shielding properties and the visible transparency were excellent, and the heat resistance and the light fastness were excellent. Further, solvent resistance was excellent.

On the other hand, in Comparative Examples, at least one of the heat resistance or the light fastness was lower than that of Examples. Further, the solvent resistance was poor.

What is claimed is:

1. A near infrared absorbing curable composition comprising:
a compound represented by Formula (1); and
a compound having a crosslinking group, 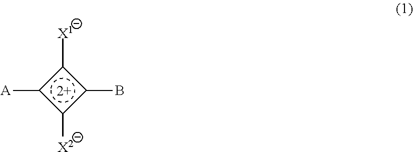
wherein in Formula (1), X¹ and X² each independently represent O, S, or a dicyanomethylene group, and A and B each independently represent a group represented by Formula (2), and 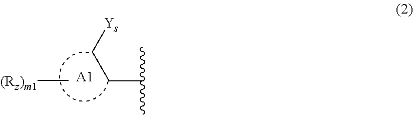
in Formula (2), a wave line represents a binding site of Formula (1), Y_{S} represents a group having active hydrogen, A1 represents an aromatic hydrocarbon ring or an aromatic heterocycle, R_{Z} represents a substituent, m1 represents an integer of 0 to mA, mA represents an integer representing the maximum number of R_{Z}'s which may be substituted in A1, Y_{S} may be bonded to A1 or R_{Z} to form a ring, and R_{Z} may be bonded to A1 to form a ring.

2. The near infrared absorbing curable composition according to claim 1,
wherein X¹ and X² represent O.

3. The near infrared absorbing curable composition according to claim 1,
wherein A1 represents a benzene ring, a thiophene ring, a furan ring, a pyrrole ring, a pyridine ring, an azulene ring, or a fused ring including a benzene ring, a thiophene ring, a furan ring, a pyrrole ring, a pyridine ring, or an azulene ring.

4. The near infrared absorbing curable composition according to claim 1,
wherein A1 represents a benzene ring or a naphthalene ring.

5. The near infrared absorbing curable composition according to claim 1,
wherein at least one of A or B is represented by Formula (3), Formula (4), Formula (5), or Formula (6), 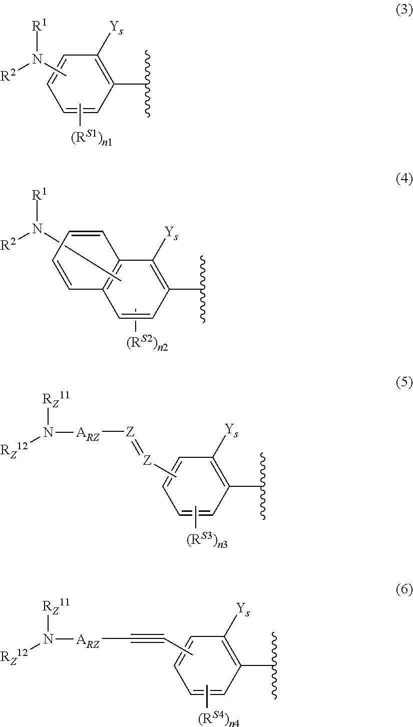
in Formula (3), a wave line represents a binding site of Formula (1), Y_{S} represents a group having active hydrogen, R¹ and R² each independently represent an alkyl group, an aryl group, or a heteroaryl group, R^{S1} represents a substituent, n1 represents an integer of 0 to 3, and R¹ and R² may be bonded to each other to form a ring or may be bonded to a benzene ring bonded to Y_{S} to form a ring,
in Formula (4), a wave line represents a binding site of Formula (1), Y_{S} represents a group having active hydrogen, R^{S2}'s each independently represent a substituent, n2 represents an integer of 0 to 5, and R¹ and R² may be bonded to each other to form a ring or may be bonded to a naphthalene ring bonded to Y_{S} to form a ring,
in Formula (5), a wave line represents a binding site of Formula (1), Y_{S} represents a group having active hydrogen, Z represents CR or N, R represents a hydrogen atom, an alkyl group, a halogen atom, or a cyano group, A_{RZ} represents an aromatic hydrocarbon ring or an aromatic heterocycle, R_{Z}¹¹ and R_{Z}¹² each independently represent a hydrogen atom, an alkyl group, an alkenyl group, an alkynyl group, an aryl group, a heteroaryl group, or an aralkyl group, R_{Z}¹¹ and R_{Z}¹² may be bonded to each other to form a ring, R^{S3} represents a substituent, and n3 represents an integer of 0 to 3, and
in Formula (6), a wave line represents a binding site of Formula (1), Y_{S} represents a group having active hydrogen, A_{RZ} represents an aromatic hydrocarbon ring or an aromatic heterocycle, R_{Z}¹¹ and R_{Z}¹² each independently represent a hydrogen atom, an alkyl group, an alkenyl group, an alkynyl group, an aryl group, a heteroaryl group, or an aralkyl group, R_{Z}¹¹ and R_{Z}¹² may be bonded to each other to form a ring, R^{S4} represents a substituent, and n4 represents an integer of 0 to 3.

6. The near infrared absorbing curable composition according to claim 1,
wherein at least one of A or B is represented by Formula (3-1), Formula (5-1), or Formula (6-1), 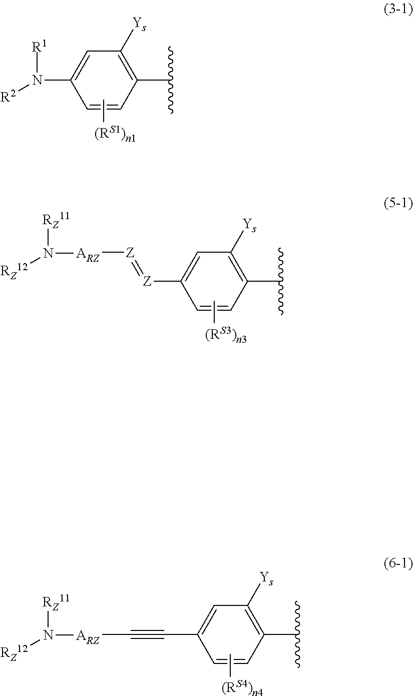
in Formula (3-1), a wave line represents a binding site of Formula (1), Y_{S} represents a group having active hydrogen, R¹ and R² each independently represent an alkyl group, an aryl group, or a heteroaryl group, R^{S1} represents a substituent, n1 represents an integer of 0 to 3, and R¹ and R² may be bonded to each other to form a ring or may be bonded to a benzene ring bonded to Y_{S} to form a ring,
in Formula (5-1), a wave line represents a binding site of Formula (1), Y_{S} represents a group having active hydrogen, Z represents CR or N, R represents a hydrogen atom, an alkyl group, a halogen atom, or a cyano group, A_{RZ} represents an aromatic hydrocarbon ring or an aromatic heterocycle, R_{Z}¹¹ and R_{Z}¹² each independently represent a hydrogen atom, an alkyl group, an alkenyl group, an alkynyl group, an aryl group, a heteroaryl group, or an aralkyl group, R_{Z}¹¹ and R_{Z}¹² may be bonded to each other to form a ring, R^{S3} represents a substituent, and n3 represents an integer of 0 to 3, and
in Formula (6-1), a wave line represents a binding site of Formula (1), Y_{S} represents a group having active hydrogen, A_{RZ} represents an aromatic hydrocarbon ring or an aromatic heterocycle, R_{Z}¹¹ and R_{Z}¹² each independently represent a hydrogen atom, an alkyl group, an alkenyl group, an alkynyl group, an aryl group, a heteroaryl group, or an aralkyl group, R_{Z}¹¹ and R_{Z}¹² may be bonded to each other to form a ring, R^{S4} represents a substituent, and n4 represents an integer of 0 to 3.

7. The near infrared absorbing curable composition according to claim 1,
wherein at least one of A or B is represented by Formula (3-1-1) or Formula (3-1-2), 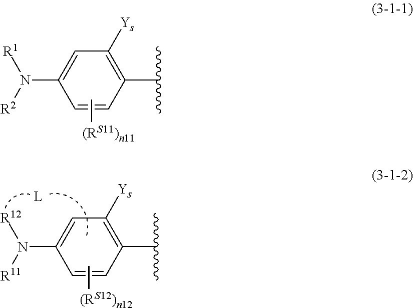
in Formula (3-1-1), a wave line represents a binding site of Formula (1), Y_{S} represents a group having active hydrogen, Ar¹ and Ar² each independently represent an aryl group or a heteroaryl group, R^{S11} represents a substituent, n11 represents an integer of 0 to 2, and Ar¹ and Ar² may be bonded to each other to form a ring or may be bonded to a benzene ring bonded to Y_{S} to form a ring, and
in Formula (3-1-2), a wave line represents a binding site of Formula (1), Y_{S} represents a group having active hydrogen, R¹¹ represents an alkyl group, an aryl group, or a heteroaryl group, R¹² represents an alkylene group, L represents a divalent linking group through which R¹² and a benzene ring are bonded to form a ring, R^{S12} represents a substituent, n12 represents an integer of 0 to 2, and R¹¹ may be bonded to a benzene ring bonded to Y_{S} to form a ring.

8. The near infrared absorbing curable composition according to claim 7,
wherein at least one of A or B is represented by Formula (3-1-1).

9. The near infrared absorbing curable composition according to claim 1,
wherein Y_{S} is represented by Formula (Y-1),
—W—Z (Y-1),
W represents a single bond or a divalent linking group,
Z represents —OH, —NHCOR^{x1}, —NHCONR^{x1}R^{x2}, —NHCOOR^{x1}, —NHSO₂R^{x1}, or —NHBR^{x1}R^{x2},
R^{x1} and R^{x2} each independently represent a substituent, and
R^{x1} and R^{x2} may be bonded to each other to form a ring or may be bonded to an aromatic hydrocarbon ring or an aromatic heterocycle bonded to Y_{S} to form a ring.

10. The near infrared absorbing curable composition according to claim 1,
wherein Y_{S} is represented by Formula (Y-2), and
—NH-T (Y-2),
T represents a group having a Hammett substituent constant σp value of 0.3 or higher.

11. The near infrared absorbing curable composition according to claim 10,
wherein T represents —CO—R^{x3}, —CONH—R^{x3}, —COO—R^{x3}, or —SO₂—R^{x3}, and
R^{x3} represents a substituent.

12. The near infrared absorbing curable composition according to claim 10,
wherein T represents —SO₂—R^{x3}, and
R^{x3} represents a substituent.

13. The near infrared absorbing curable composition according to claim 11,
wherein R^{x3} represents a group having a fluorine atom.

14. The near infrared absorbing curable composition according to claim 1,
wherein the compound represented by Formula (1) is a compound represented by Formula (1A), 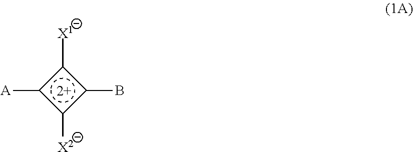
in Formula (1A), X¹ and X² each independently represent O, S, or a dicyanomethylene group, A and B each independently represent a group represented by Formula (2), and at least one of A or B represents a group represented by Formula (10), 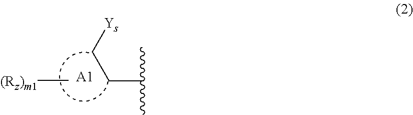
in Formula (2), a wave line represents a binding site of Formula (1A), Y_{S} represents a group having active hydrogen, A1 represents an aromatic hydrocarbon ring or an aromatic heterocycle, R_{Z} represents a substituent, m1 represents an integer of 0 to mA, mA represents an integer representing the maximum number of R_{Z}'s which may be substituted in A1, Y_{S} may be bonded to A1 or R_{Z} to form a ring, and R_{Z} may be bonded to A1 to form a ring, and 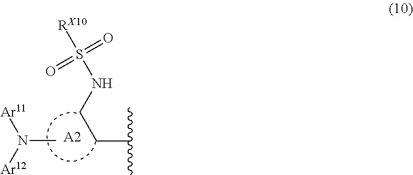
in Formula (10), a wave line represents a binding site of Formula (1A), A2 represents an aromatic hydrocarbon ring or an aromatic heterocycle, Ar¹¹ and Ar¹² each independently represent an aryl group or a heteroaryl group, R^{X10} represents a substituent, and Ar¹¹ and Ar¹² may be bonded to each other to form a ring or may be bonded to A2 to form a ring.

15. The near infrared absorbing curable composition according to claim 1,
wherein the compound having a crosslinking group is at least one selected from the group consisting of a compound which has a group having an ethylenically unsaturated bond, a compound having a cyclic ether group, a compound having an alkoxysilyl group, and a compound having a chlorosilyl group.

16. The near infrared absorbing curable composition according to claim 1, further comprising:
at least one selected from the group consisting of a polyfunctional thiol, an alcohol, an amine, and a carboxylic acid.

17. A cured film which is formed using the near infrared absorbing curable composition according to claim 1.

18. The cured film according to claim 17, which is an infrared cut filter.

19. A solid image pickup element comprising:
the cured film according to claim 17.

20. An infrared absorber which is represented by Formula (1A), 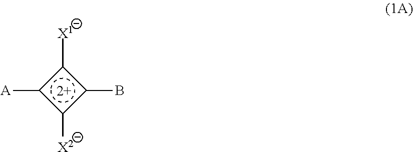
in Formula (1A), X¹ and X² each independently represent O, S, or a dicyanomethylene group, A and B each independently represent a group represented by Formula (2), and at least one of A or B represents a group represented by Formula (10), 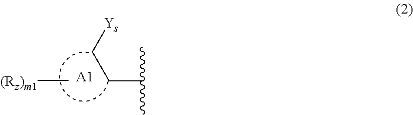
in Formula (2), a wave line represents a binding site of Formula (1A), Y_{S} represents a group having active hydrogen, A1 represents an aromatic hydrocarbon ring or an aromatic heterocycle, R_{Z} represents a substituent, m1 represents an integer of 0 to mA, mA represents an integer representing the maximum number of R_{Z}'s which may be substituted in A1, Y_{S} may be bonded to A1 or R_{Z} to form a ring, and R_{Z} may be bonded to A1 to form a ring, and 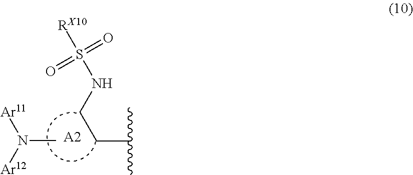
in Formula (10), a wave line represents a binding site of Formula (1A), A2 represents an aromatic hydrocarbon ring or an aromatic heterocycle, Ar¹¹ and Ar¹² each independently represent an aryl group or a heteroaryl group, R^{X10} represents a substituent, and Ar¹¹ and Ar¹² may be bonded to each other to form a ring or may be bonded to A2 to form a ring.

21. A compound which is represented by Formula (1A), 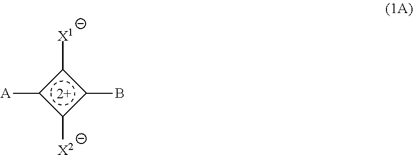
in Formula (1A), X¹ and X² each independently represent O, S, or a dicyanomethylene group, A and B each independently represent a group represented by Formula (2), and at least one of A or B represents a group represented by Formula (10), 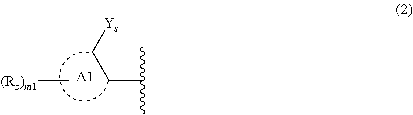
in Formula (2), a wave line represents a binding site of Formula (1A), Y_{S} represents a group having active hydrogen, A1 represents an aromatic hydrocarbon ring or an aromatic heterocycle, R_{Z} represents a substituent, m1 represents an integer of 0 to mA, mA represents an integer representing the maximum number of R_{Z}'s which may be substituted in A1, Y_{S} may be bonded to A1 or R_{Z} to form a ring, and R_{Z} may be bonded to A1 to form a ring, and 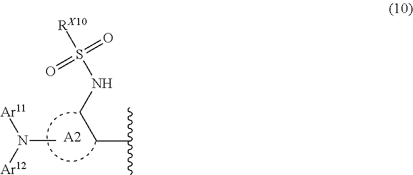
in Formula (10), a wave line represents a binding site of Formula (1A), A2 represents an aromatic hydrocarbon ring or an aromatic heterocycle, Ar¹¹ and Ar¹² each independently represent an aryl group or a heteroaryl group, R^{X10} represents a substituent, and Ar¹¹ and Ar¹² may be bonded to each other to form a ring or may be bonded to A2 to form a ring.

22. The compound according to claim 21,
wherein R^{X10} represents a group having a fluorine atom.
